(19) United States
(12) Patent Application Publication
(10) Pub. No.: US 2024/0417747 A1
(43) Pub. Date: Dec. 19, 2024

(54) Controlling Citrus Huanglongbing (HLB) by Mitigating Cell Death of the Phloem Tissues Caused by Candidatus Liberibacter Asiaticus (CLas) By Suppressing Reactive Oxygen Species (ROS)

(71) Applicant: UNIVERSITY OF FLORIDA RESEARCH FOUNDATION, INCORPORATED, Gainesville, FL (US)

(72) Inventor: Nian WANG, Gainesville, FL (US)

(21) Appl. No.: 18/707,248

(22) PCT Filed: Nov. 3, 2022
(86) PCT No.: PCT/US22/79250

§ 371 Date: May 3, 2024

Related U.S. Application Data
(60) Provisional application No. 63/301,622, filed on Jan. 21, 2022.
(60) Provisional application No. 63/275,064, filed on Nov. 3, 2021.

(51) Int. Cl. C12N 15/82; C12N 9/02; C12N 9/22

(52) U.S. Cl. CPC C12N 15/8281; C12N 9/0036; C12N 9/22; C12N 15/8201; C12N 2310/20; C12Y 106/03001

(57) ABSTRACT

It is presented that citrus Huanglongbing (HLB), caused by phloem-colonizing Candidatus Liberibacter asiaticus (CLas), is an immune-mediated disease. CLas infection of Citrus sinensis stimulated systemic and chronic immune response in the phloem tissues including reactive oxygen species (ROS) production. Methods and compositions for transfecting plants and regenerating plants to express antioxidant enzymes or shut down genes to reduce adverse effects of CLas infection are provided.

### GOVERNMENT SUPPORT

This invention was made with government support under Grant Nos. 2018-70016-27412 and 2022-70029-38471 awarded by United States Department of Agriculture, National Institute of Food and Agriculture. The government has certain rights in the invention.

### BACKGROUND

Both plants and animals utilize the immune system to fight off pathogens. However, some human diseases are mediated by the immune response. Immune-mediated diseases include diseases due to inflammation stemming from the immune response to certain microbes and environmental antigens¹. For example, inflammatory bowel disease is disorders that involve chronic inflammation of the digestive tract. Bacterial pathogens can instigate chronic inflammation that leads to diseases beyond the damaging effect of pathogenicity factors². In addition, autoimmune diseases, such as allergic diseases, and allergic asthma are also immune mediated diseases.

The damaging effect of plant diseases has been assumed to directly result from the impact of pathogenicity factors of corresponding pathogens 3. Common pathogenicity factors include effectors, toxins, cell wall degrading enzymes, and biofilm that are directly responsible for causing disease symptoms. For instance, the transcriptional activator-like effector PthA4 is responsible for the hypertrophy and hyperplasia symptoms of citrus canker caused by Xanthomonas citri subsp. citri⁴. Xylem blockage caused by biofilm of Xylella fastidiosa is known to lead to the wilting of grapevine plants with Pierce's disease⁵. Citrus Huanglongbing (HLB, also known as citrus greening) is currently the most devastating citrus disease and causes billions of dollars economic losses worldwide annually. HLB is caused by the phloem-colonizing Candidatus Liberibacter asiaticus (CLas), Ca. L. americanus and Ca. L. africanus that are vectored by either Asian citrus psyllid (Diaphorina citri) or African citrus psyllid (Trioza erytreae)⁶. Among them, CLas is the most prevalent worldwide.

Despite its economic importance, how Ca. Liberibacter causes damages to the infected citrus plants remains poorly understood. One reason for such a delay is that HLB pathogens have not been cultured in artificial media. No pathogenicity factors have been confirmed to be responsible for the HLB symptoms including the characteristic blotchy mottle on leaves, hardened and upright small leaves, stunt growth, and root decay⁶.

### SUMMARY

Accordingly, in certain embodiments, disclosed is a plant comprising plant cells comprising a modification to a gene, or regulatory element thereof, wherein the gene encodes an antioxidant enzyme, and wherein the modification confers resistance or tolerance to Ca Liberibacter infection in the plant relative to a plant of the same variety lacking the modification, wherein the gene encoding the antioxidant enzyme optionally comprises at least one of SEQ ID NOs: 3-79. In a specific embodiment, the antioxidant enzyme is selected from a group consisting of superoxide dismutase, catalases, glutathione peroxidases, ascorbate peroxidase, glutathione reductase, and glutathione S-transferase. In a more specific embodiment, the plant is citrus. In one example, the plant modification comprises operatively linking a constitutive promoter to the gene thereby inducing overexpression of the gene. In a specific example, constitutive promoter is a 35S promoter or a phloem specific AtSUC2 promoter. Modifications include a deletion, a substitution, or an insertion. The modification may be one designed to cause activation of expression in response to CLas infection.

In another embodiment, provided is a plant comprising plant cells comprising a modification to a gene, or regulatory element thereof, wherein the gene is a respiratory burst oxidative homolog D (RbohD), and wherein the modification confers resistance or tolerance to Ca Liberibacter infection in the plant relative to a plant of the same variety lacking the modification. In a specific embodiment, the plant is citrus. The modification may include a deletion, a substitution, or an insertion of the promoter or coding region such that expression of RbohD is knocked down and/or phosphorylation of RbohD is reduced. The modification may cause a reduction of expression in response to CLas infection, or alters or eliminates a function of the regulatory element thereof.

Other embodiments pertain to a seed that produces the plant or a plant part of the plant described above. A commodity plant product, or methods of producing a commodity plant product of the plants described herein are disclosed as well.

Another embodiment pertains to a method of generating a modified plant comprising resistance or tolerance to infection by a bacterial species from the genus Ca. Liberibacter. The method involves (a) introducing a genetic modification into the genome of a plant cell, wherein the modification is to an endogenous gene or regulatory element thereof, wherein the endogenous gene encodes an antioxidant enzyme, and wherein the modification confers resistance or tolerance to Ca Liberibacter infection in the plant relative to a plant of the same variety lacking the modification; and (b) regenerating the modified plant from said plant cell or a progenitor cell thereof, wherein said the plant comprises said modification, wherein the gene encoding the antioxidant enzyme optionally comprises at least one of SEQ ID NOs: 3-79. In a specific embodiment, the plant is citrus. Further embodiments relate to a method of generating a modified plant comprising resistance or tolerance to infection by a bacterial species from the genus Ca. Liberibacter. The method includes the steps of (a) introducing a genetic modification into the genome of a plant cell, wherein the modification is to an RbohD gene or regulatory element thereof, wherein the modification confers resistance or tolerance to Ca Liberibacter infection in the plant relative to a plant of the same variety lacking the modification; and (b) regenerating the modified plant from said plant cell or a progenitor cell thereof, wherein said the plant comprises said modification. In a specific embodiment is citrus. In a more specific embodiment, the modification is to the promoter of the RbohD gene so as to reduce expression in response to CLas infection, or a modification of the coding region that knocks-down expression of the RbohD gene. The modification can be effectuated by a genome-editing technique, such as a nuclease, wherein the nuclease introduces a single-strand DNA break or a double-strand DNA break, a TALEN, a ZFN, meganuclease, or a CRISPR/Cas system.

Plants produced by the methods above are also provided.

Another embodiment relates to a method of increasing resistance or tolerance of a citrus plant to infection by a bacterial species from the genus Ca. Liberibacter that involves introducing an expression vector into a plant cell of the plant, wherein the expression vector comprises a gene, or regulatory element thereof, optionally with at least one modification, wherein the gene encodes a citrus antioxidant enzyme and wherein the gene encoding the antioxidant enzyme optionally comprises at least one of SEQ ID NOs: 3-79. In a specific embodiment, the plant is citrus. The modification may involve adding a constitutive promoter operatively linked to the gene thereby inducing overexpression of the gene. In certain examples, the constitutive promoter is a 35S promoter or a phloem specific AtSUC2 promoter. The modification may include a deletion, a substitution, or an insertion, and typically causes activation of expression in response to CLas infection. In one example, the expression vector is delivered to the plant cell via a CTV vector.

Another embodiment pertains to a method of generating a modified plant comprising resistance or tolerance to infection by a bacterial species from the genus Ca. Liberibacter; that involves (a) introducing a genetic modification into the genome of a plant cell, wherein the modification is to an RbohB gene or regulatory element thereof or RbohF gene or regulatory element thereof, wherein the modification confers resistance or tolerance to Ca Liberibacter infection in the plant relative to a plant of the same variety lacking the modification; and (b) regenerating the modified plant from said plant cell or a progenitor cell thereof, wherein said the plant comprises said modification. In certain examples, the plant is citrus, and the RbohB gene or RbohF genes are Cs3g14240 or Cs5g02940, respectively. Plants produced by the above method are also provided.

A further embodiment relates to a plant comprising plant cells comprising a modification to a gene, or regulatory element thereof, wherein the gene is a respiratory burst oxidative homolog B (RbohB) or respiratory burst oxidative homolog F (RbohF), and wherein the modification confers resistance or tolerance to Ca Liberibacter infection in the plant relative to a plant of the same variety lacking the modification. In one example, the plant is citrus. The modification may include a deletion, a substitution, or an insertion of the promoter or coding region such that expression of RbohB or RbohF is knocked down.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 shows CLas infection causes ROS production, callose deposition, and starch accumulation in young citrus leaves. (A) Quantification of H₂O₂ in young leaves of two-year-old HLB positive and healthy C. sinensis ‘Valencia’ trees. n=4. (B) Quantification of callose depositions of young leaves by staining with aniline blue and observed under an epifluorescence microscope. Data shown are mean±SD (standard deviation), n=10. Callose depositions were counted from ten leaves from five trees. (C) Starch content in young flush. Data shown are mean±SD; n=4. HLB positive and healthy two-year-old C. sinensis ‘Valencia’ trees were trimmed to trigger young flush in the greenhouse. qPCR was conducted to quantify CLas titers in newly emerged leaves at different time points after bud initiation. Data shown are mean±SD; n=4. Experiment was repeated twice with similar results. *, **, and *** indicate P value <0.05, 0.01, and 0.001, respectively, compared with healthy young leaves and calculated using Student's t-test.

FIG. 2 shows Transmission Electron Microscopy (TEM) analysis of asymptomatic young leaves of CLas infected Citrus sinensis. (A) Heathy leaf of C. sinensis ‘Valencia’. CC (companion cells): typical healthy companion cells with large central nucleus, dense cytoplasm, numerous mitochondria. SE (sieve element 1-3): typical sieve elements showing parietal “s” plastids, centrally distributed p protein, and lateral sieve plate showing minimum callose. SE (4-5): developing sieve elements showing plastids with no starch, intact central vacuole and non-dispersed phloem protein. (B) Asymptomatic young leaf of CLas infected C. sinensis. CC undergoing cell death. (C, D, and E) Asymptomatic young leaves showing cell death of sieve element cells and companion cells, but not parenchyma cells (PC). Black * indicates cell death of sieve element cells. White * indicates cell death of companion cells. (D) Enlarged section of C showing both intact sieve element and companion cells as well as sieve element and companion cells undergoing cell death. (E) Enlarged section of D showing intact sieve element cells containing CLas.

FIG. 3 shows Candidatus Liberibacter asiaticus (CLas) induces ROS production and cell death in phloem tissues of C. sinensis. (A) Trypan blue staining assay to detect cell death in leaves of CLas infected C. sinensis ‘Valencia’ trees. (B) Determination of H₂O₂ concentration in CLas negative or positive leaves showing different symptoms. Mean and SD are shown. (C and D) H₂O₂(C) and ion leakage (D) assays of exudates extracted from phloem enriched bark tissues. Statistical differences were analyzed using one way ANOVA with Bonferroni Correction (P<0.05) for B and using Student's t-test for C and D. Different letters above the columns indicate statistical differences (P<0.05). ND: non-detected. For A and B: AS: asymptomatic. MS: Mildly symptomatic. SS: Severely symptomatic. AS, MS, and SS were collected from HLB-positive trees. For C and D, H: stems of spring sprouts were collected from healthy branches of CLas-negative plants. S: stems of spring sprouts were collected from branches with HLB symptomatic leaves of CLas-positive plants. Each experiment contains four biological replicates. The experiments were repeated at least twice with similar results. CLas titers were determined by qPCR as shown by Ct values with lower Ct values indicating higher bacterial titers.

FIG. 4 shows CLas infection induces callose deposition, H₂O₂ production and cell death in the phloem tissues of C. sinensis. (A-F) C. sinensis ‘Hamlin’ leaf samples were fixed with FAA solution overnight, sectioned and stained with 0.005% aniline blue solution prior to analysis. Xylem is marked with an X, and callose deposition is indicated with arrowheads. Asymptomatic samples: (A) Petiole, (B) Midrib, (C) Lamina; Symptomatic samples: (D) Petiole, (E) Midrib, and (F) Lamina. Pictures are representatives of 12 replicates. ND: Non-detected. (G) schematic representation of samples used for callose deposition assays (A-F). CLas titers for each section were quantified by qPCR. (H-J) H₂O₂ production in the phloem tissues. Healthy (H) and CLas infected (I) C. sinensis ‘Valencia’ bark tissues visualized with 2′,7′-dichlorodihydrofluorescein diacetate (H₂DCFDA) under a confocal laser microscope. CLas infected (J) C. sinensis ‘Valencia’ bark without H2DCFDA was used as a control. “+” indicates with H DCFDA treatment. “−” indicates without H₂DCFDA treatment. (K-L) Effect of killing CLas with streptomycin on H₂O₂ concentration (K) and cell death (L) in the phloem tissues. CLas positive five-year-old sweet orange trees were trunk-injected with streptomycin. Non-treatment was used as the negative control. The tests were conducted 7 days after trunk injection of streptomycin. Four biological replicates were used. * indicates significant statistical difference (P<0.05) based on Student's t-test.

FIG. 5 shows ROS is responsible for cell death of the phloem tissues of CLas infected citrus. (A and B) H₂O₂ kills protoplast cells of C. sinensis. (A) Freshly prepared protoplast cells of C. sinensis were treated with different concentrations of H₂O₂ with or without the antioxidant uric acid (0.2 mM) for 24 h and tested for viability via fluorescein diacetate (FDA) staining. (B) Quantification of viable protoplast cells in different treatments of A. (C and D) HLB-positive plants were treated with antioxidants via foliar spay weekly for six weeks. The exudates extracted from phloem enriched bark tissues were used for detection of H₂O₂ and ion leakage. (C) Uric acid (1.8 mM) and rutin (0.6 mM) reduce H₂O₂ concentration triggered by CLas in the phloem tissue. (D) Uric acid (1.8 mM) and rutin (0.6 mM) reduce ion leakages in the phloem tissues infected by CLas. Statistical differences were analyzed using one way ANOVA with Bonferroni Correction (P<0.05). Each experiment contains four biological replicates. Different letters above the columns indicate statistical differences (P<0.05). Ct values of CLas of the tested samples were indicated.

FIG. 6 shows immunoregulator gibberellin (GA) suppresses HLB development. (A and B) GA suppresses ROS mediated cell death. HLB-positive C. sinensis trees were treated with GA via foliar spray weekly for six weeks. The exudates extracted from phloem enriched bark tissues were used for detection of H₂O₂(A) and ion leakage (B). n=4. Ct values of CLas of the tested samples were indicated. (C and D) GA suppresses cell death of C. sinensis protoplast cells. Freshly prepared protoplast cells of C. sinensis were treated with H₂O₂ with or without GA (5 mg/L) for 24 h and tested for viability via fluorescein diacetate (FDA) staining. Statistical differences (A, B, and D) were analyzed using one way ANOVA with Bonferroni Correction (P<0.05). Different letters above the columns indicate statistical differences (P<0.05). (E-G) Foliar spray of GA suppresses HLB symptoms. C. sinensis ‘Valencia’ blocks were treated with GA (1,247 ppm) in November 2020. Nearby blocks of C. sinensis ‘Valencia’ that were not treated with GA were used as negative controls. Symptoms, HLB disease incidence and ratio of symptomatic leaves/total leaves were investigated in June 2021. (E) Representative whole trees. (F) Representative sections. (G) HLB disease incidence and ratio of symptomatic leaves vs total leaves in different treatments. Pictures were taken at the same day in June 2021. * indicates P value <0.05 based on Student's t-test.

FIG. 7 shows temporal expression of immune-related genes in response to CLas infection of young flush of C. sinensis. The reverse transcription-quantitative PCR (RT-qPCR) analysis was conducted using young leaves from three two-year-old HLB positive Valencia sweet orange trees compared with that of three healthy Valencia trees with one tree as one biological replicate.

FIG. 8 shows TEM observation of the midribs of mature leaves of C. sinensis trees grown in the field that are HLB positive. (A and B) Midvein of asymptomatic mature leaf. (C and D) Midvein of symptomatic mature leaf. CLas titers were indicated by Ct values for the samples used. Scale bar for each picture is included. SE: sieve element. DSE: dead sieve element cells. PP: parenchyma cells. CI: calcium oxalate crystal idioblas.

FIG. 9 shows TEM observation of the stem tissues of HLB positive C. sinensis trees grown in the field. Stems were collected from branches without HLB symptoms (A and B) and branches with HLB symptoms (C and D). Scale bar for each picture is included. SE: sieve element. DSE: dead sieve element cells. PP: parenchyma cells. CI: calcium oxalate crystal idioblas. CLas titers in the tested samples were indicated by Ct values.

FIG. 10 shows comparison of ion leakage activities of leaves of CLas-negative and CLas-infected sweet orange trees. H: healthy leaves. AS: asymptomatic leaves. MS: leaves with mild symptoms. SS: leaves with severe symptoms. Healthy leaves were collected from CLas-free sweet orange plants. AS, MS, and SS were collected from CLas positives sweet orange trees in the groves. For statistical significance tests, Student's t-test was conducted (n=9). Mean and standard deviation are shown. P values for comparisons against CLas negative samples were shown above each column.

FIG. 11 shows H₂O₂ kills protoplast cells of C. sinensis. Freshly prepared protoplast cells of C. sinensis were treated with different concentrations of H₂O₂ for 24 h and tested for viability via fluorescein diacetate (FDA) staining. Each experiment contains three biological replicates. Mean and SD were shown. Statistical differences were analyzed using one way ANOVA with Bonferroni Correction (P<0.05). Different letters above the columns indicate statistical differences (P<0.05).

FIG. 12 shows the expression profile of ROS-related genes in response to CLas infection in 9 previous studies. Affiliation of each gene is indicated in brackets. Orange denotes “higher in CLas infected than CLas negative samples” while blue denotes “higher in CLas negative than CLas infected samples”. The asterisk denotes P value <0.01; the plus sign denotes P value <0.05. The information about the 9 previous studies is listed in Table 2.

FIG. 13 shows the expression profile of immune related genes, including PR, MAPK and NBS-LRR genes in response to CLas infection in 9 previous studies. Affiliation of each gene is indicated in brackets. Orange denotes “higher in CLas infected than CLas negative samples” while blue denotes “higher in CLas negative than CLas infected samples”. The asterisk denotes P value <0.01; the plus sign denotes P value <0.05. (A) PR genes, and MAPK genes. (B and C) NBS-LRR genes. The information about the 9 previous studies is listed in Table 2.

FIG. 14 shows effect of GA and antioxidants treatment on HLB symptoms. To test the effect of GA treatment on HLB symptoms, HLB positive C. sinensis ‘Valencia’ trees were treated with GA and antioxidants (uric acid and rutin) via foliar spray weekly. Representative branches were selected to demonstrate symptom changes.

FIG. 15 shows gibberellin (GA) treatment of C. sinensis suppresses HLB. C. sinensis ‘Vernia’ blocks were treated with GA (1247 ppm) in November 2020. Nearby blocks of C. sinensis ‘Vernia’ that were not treated with GA were used as negative controls. Symptoms, HLB disease incidence and ratio of symptomatic leaves/total leaves were investigated in June 2021. (A) Representative whole trees. (B) Representative sections. (C) HLB disease incidence and ratio of symptomatic leaves vs total leaves in different treatments. Pictures were taken at the same day in June 2021. * indicates P value <0.05 based on Student's t-test.

FIG. 16 shows growth performance of citrus trees (two cultivars: Valencia and Vernia) treated by GA in commercial groves in FL. C. sinensis blocks were treated with GA (1247 ppm) in November 2020. Nearby blocks of C. sinensis that were not treated with GA were used as negative controls. Trees were investigated in June 2021. (A) Trunk diameter measured at ˜20 cm above the ground. (B) Tree height above the ground from the soil surface to the apical point of the plant. (C) Canopy volume of the GA treated and untreated control trees. Data shown are means of 10 replicated trees (n=10). The error bars are standard deviations. P values of Student's two-tailed t tests were presented and a significant difference between GA treated, and untreated control trees was determined at P<0.05.

FIG. 17 shows expression profiling of ROS related genes between GA and non-GA (nGA) treated Citrus sinensis protoplast cells in the presence of 1.8 mM H₂O₂. The genes include ROS related genes encoding ascorbate peroxidase, glutathione reductase, catalase, dehydroascorbate reductase (DHAR), superoxide dismutases (SOD), glutathione peroxidase (GPX), ferritin and blue copper protein, NADPH oxidases, alternative oxidase, peroxiredoxin, thioredoxins, glutaredoxin, and non-enzymatic antioxidants-related genes. The asterisk denotes P value <0.01; the plus sign denotes P value <0.05. The gene expression value was calculated using RPKM method. Scale indicates expression value of each gene after row normalization by removing the mean (centering) and dividing by the standard deviation (scaling). The color scale represents log fold change of GA vs nGA.

FIG. 18 shows viability of Citrus sinensis ‘Hamlin’ suspension culture cells treated with H₂O₂. The suspension culture was treated with H₂O₂ for 24 hours. Each sample was stained with fluorescein diacetate (stain only living cells, green color) and propidium iodide (stain only dead cells, orange to red color). Representative pictures are shown.

FIG. 19 shows that expression of NADPH oxidase genes in response to CLas infection. (A) Expression analyses of NADPH oxidase genes (RBOHB, RBOHD, and RBOHF) in response to CLas infection in the greenhouse via qRT-PCR. The housekeeping gene GAPDH encoding glyceraldehyde-3-phosphate dehydrogenase-C was used as an endogenous control. Error bars indicate standard deviation of mean (four biological replicates). P value was calculated by Student's t-test. (B) Expression analyses of NADPH oxidase genes (RBOHB, RBOHD, and RBOHF) in response to CLas infection in the field via qRT-PCR. The housekeeping gene GAPDH encoding glyceraldehyde-3-phosphate dehydrogenase-C was used as an endogenous control. Error bars indicate standard deviation of mean (four biological replicates). P value was calculated by Student's t-test.

FIG. 20 shows that ROS levels in CLas-positive stems were reduced by NADPH oxidase inhibitor diphenyleneiodonium (DPI). HLB positive branches from the summer flush of Valencia sweet orange in the field were collected and then soaked in DPI solution (25 μM) or water (control). After 48 h treatment, phloem-enriched bark tissues were collected for H₂O₂ concentration measurement. Eight biological replicates were used for this test. * indicates p<0.01 (Student's t-test). The experiment was repeated twice with similar results.

FIG. 21 shows a comparative analysis of RbohD sequences from A. thaliana and C. sinensis. Phosphorylation sites are also indicted in the blue boxes.

Table 1 shows overexpression of CLas proteins containing Sec-section signals and other predicated virulence factors in Arabidopsis, Citrus and Nicotiana.

Table 2 shows transcriptomic studies of sweet orange in response to CLas infection that were used for GO enrichment analysis in this study.

Table 3 shows GO enrichment analysis of DEGs of Citrus sinensis in response to CLas infection based on nine different studies as specified in Table 2.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “about,” as used herein, means plus or minus 20 percent of the recited value, so that, for example, “about 0.125” means 0.125±0.025, and “about 1.0” means 1.0±0.2.

The term “applying,” “application,” “administering,” “administration,” and all their cognates, as used herein, refers to any method for contacting the plant with the glyphosate compositions discussed herein. Administration generally is achieved by application of the glyphosate, in a vehicle compatible with the plant to be treated (i.e., a botanically compatible vehicle or carrier), such as an aqueous vehicle, to the plant. Any application means can be used, however preferred application is foliar spraying. Other methods include application to the soil surrounding the plant, by injection, soaking or spraying, so that the applied composition preferably comes into contact with the phloem of the plant.

The term “botanically acceptable carrier/vehicle” or “botanically compatible carrier/vehicle,” as used herein, refers to any non-naturally occurring vehicle, in liquid, solid or gaseous form which is compatible with use on a living plant and is convenient to contain a substance or substances for application of the substance or substances to the plant, its leaves or root system, its seeds, the soil surrounding the plant, or for injection into the trunk, or any known method of application of a compound to a living plant, preferably a crop plant, for example a citrus tree, citrus seedling, and the like. Useful vehicles can include any known in the art, for example liquid vehicles, including aqueous vehicles, such as water, solid vehicles such as powders, granules or dusts, or gaseous vehicles such as air or vapor. Any vehicle which can be used with known devices for soaking, drenching, injecting into the soil or the plant, spraying, dusting, or any known method for applying a compound to a plant, is contemplated for use with embodiments of the invention. Typical carriers and vehicles contain inert ingredients such as fillers, bulking agents, buffers, preservatives, anti-caking agents, pH modifiers, surfactants, soil wetting agents, adjuvants, and the like. Suitable carriers and vehicles within this definition also can contain additional active ingredients such as plant defense inducer compounds, nutritional elements, fertilizers, pesticides, and the like.

The term “Citrus” or “citrus,” as used herein, refers to any plant of the genus Citrus, family Rutaceae, and includes Citrus maxima (Pomelo), Citrus medica (Citron), Citrus micrantha (Papeda), Citrus reticulata (Mandarin orange), Citrus trifolata (trifoliate orange), Citrus japonica (kumquat), Citrus australasica (Australian Finger Lime), Citrus australis (Australian Round lime), Citrus glauca (Australian Desert Lime), Citrus garrawayae (Mount White Lime), Citrus gracilis (Kakadu Lime or Humpty Doo Lime), Citrus inodora (Russel River Lime), Citrus warburgiana (New Guinea Wild Lime), Citrus wintersii (Brown River Finger Lime), Citrus halimii (limau kadangsa, limau kedut kera) Citrus indica (Indian wild orange), Citrus macroptera, and Citrus latipes. Hybrids also are included in this definition, for example Citrus x aurantiifolia (Key lime), Citrus x aurantium (Bitter orange), Citrus x latifolia (Persian lime), Citrus x limon (Lemon), Citrus x limonia (Rangpur), Citrus x paradisi (Grapefruit), Citrus x sinensis (Sweet orange), Citrus x tangerina (Tangerine), Poncirus trifoliata x C. sinensis (Carrizo citrange), C. paradisi “Duncan” grapefruit x Pondirus trifoliate (Swingle citrumelo), and any other known species or hybrid of genus Citrus. Citrus known by their common names include, Imperial lemon, tangelo, orangelo, tangor, kinnow, kiyomi, Minneola tangelo, oroblanco, sweet orange, ugli, Buddha's hand, citron, lemon, orange, bergamot orange, bitter orange, blood orange, calamondin, clementine, grapefruit, Meyer lemon, Rangpur, tangerine, and yuzu, and these also are included in the definition of citrus or Citrus.

The term “citrus plant,” as used herein, refers to a mature plant, seed, cutting, embryo, seedling, and/or sapling, and the like of any citrus variety.

The term “effective amount” or “therapeutically effective amount,” as used herein, with respect to treatment means any amount of the glyphosate compound or a composition containing this compound, which reduces the symptoms of HLB disease in a citrus plant or population of citrus plants, reduces the amount of pathogenic bacteria in a citrus plant or population of citrus plants, improves health, growth or productivity of the plant, or which reduces the effects, titer or symptoms of the plant disease, or prevents worsening of the plant disease, symptoms or infection of the plant. This term includes an amount effective to increase seed germination of a plant or a plant population, to increase the speed of seed germination of a plant or a plant population, to increase growth rates of a plant or a plant population, to increase crop yield of a plant or plant population, increase crop quality in a plant or plant population, reduce the plant pathogen titer, to inhibit plant pathogen growth, to reduce the percent of infected plants in a plant population, to reduce the percent of plants showing disease symptoms in a plant or plant population, to reduce the disease symptom severity rating or damage rating of a plant or plant population, to reduce average pathogen population or titer in a plant or plant population by about 2%, about 5%, about 10%, about 20%, about 30%, about 40%, about 50%, about 60%, about 70%, about 75%, about 80%, about 85%, about 90%, or more, compared to plants or a plant population not treated with the active ingredient.

Expression: The term “expression” as used herein refers to the transcription of a particular nucleic acid sequence to produce sense or antisense RNA or mRNA, and/or the translation of an mRNA molecule to produce a polypeptide, with or without subsequent post-translational events. Expression also encompasses production of a functional nucleic acid (e.g., an RNAi, antisense molecule, ribozyme, aptamer, etc.).

Genome editing: Modifying a genome with techniques that employ targeted mutagenesis to activate DNA repair pathways. These techniques include, but are not limited to, those that utilize endonucleases to generate single-strand and double-strand DNA breaks that activate DNA repair pathways. Genome editing techniques may also comprise systems that enable targeted editing at any genomic locus. These targeting systems include, but are not limited to, polypeptides, such as, Transcription Activator-Like Effectors (TALEs) and zinc fingers (ZFs), or nucleic acids, such as, Clustered Regularly Interspaced Short Palindromic Repeats/Cas (CRISPR/CAS) single guide RNAs or NgAgo (Argonaute) single strand DNAs. As used herein, “genome editing” and “genome-engineering” are interchangeable.

Genetic modification: A DNA sequence difference, epigenetic difference, or combination thereof between two genomes of the same species in which one genome is identified as the modified genome and the other is identified as the unmodified genome and the DNA sequence or epigenetic difference is the result of applying genome modifying techniques to the unmodified genome to yield the modified genome. A genetic modification, as used herein, encompasses any insertion, deletion, or substitution of a nucleotide sequence of any size and nucleotide content, any epigenetic modification to any number of nucleotides, or a combination thereof. A genetic modification, as used herein, may also encompass introduction of one or more exogenous coding nucleic acids that do not integrate into the unmodified genome, yet are capable of autonomous replication. In certain embodiments, a modification to an endogenous gene or regulatory element thereof may be a deletion, a substitution, or an insertion that reduces expression of the endogenous gene or the polypeptide for which it encodes. In specific embodiments, the modification may be an indel, wherein the indel may cause a frameshift mutation, a missense mutation, a nonsense mutation, a neutral mutation, or a silent mutation. In specific embodiments, a modification to a regulatory element of an endogenous gene may alter or eliminate a function of the regulatory element. In further contemplated embodiments, the modification may comprise a nucleic acid sequence that provides exogenous control of endogenous gene, mRNA, or polypeptide expression levels. In specific embodiments, the modification may also disrupt a post-translational process of a polypeptide encoded by an endogenous gene. Post-translational processes in certain embodiments may be post-translational modification, protein sorting, or proteasomal degradation.

Genetically modified cell: A cell in which the endogenous genome has been genetically modified; a cell in which one or more exogenous, coding nucleic acids have been introduced that do not integrate into the genome yet are capable of autonomous replication; or a combination thereof.

Genetically modified plant: A plant comprising at least one genetically modified cell. A genetically modified plant may be regenerated from a genetically modified cell or plant part comprising genetically modified cells, and thus the genetic modification may be heritable and inherited by progeny thereof. The progeny thereof that inherit the genetic modification are also considered genetically modified plants. A genetically modified plant, as used herein, also refers to a plant in which at least one genetically modified cell is introduced to a plant or arises as a result of genetic modification techniques directly applied to the plant.

Genetic modification techniques: Any technique known to those in the art that can modify the genome of a cell including, but not limited to, genome editing, site-specific genetic recombination, epigenetic modifications, and genetic transformation.

Genetic Transformation: A process of introducing a DNA sequence or construct (e.g., a vector or expression cassette) into a cell or protoplast in which that exogenous DNA is incorporated into a chromosome or is capable of autonomous replication.

The term “gibberellins” refers to tetracyclic diterpene acids plant hormones that regulate various developmental processes, including stem elongation, germination, dormancy, flowering, flower development, and leaf and fruit senescence. All known gibberellins are diterpenoid acids that are synthesized by the terpenoid pathway in plastids and then modified in the endoplasmic reticulum and cytosol until they reach their biologically-active form. Gibberellins include but are not limited to the following: gibberellic acid, gibberellin A3, gibberellin A4, gibberellin A7, gibberellin A13, iso-gibberellin A7, and iso-gibberellin A7 methyl ester.

Heterologous: A sequence which is not normally present in a given host genome in the genetic context in which the sequence is currently found. In this respect, the sequence may be from another species, organism, plant, tree, or variety, or may be native to the host genome, but be rearranged with respect to other genetic sequences within the host sequence. For example, a regulatory sequence may be heterologous in that it is linked to a different coding sequence relative to the native regulatory sequence. In addition, a particular recombinant DNA molecule may be heterologous with respect to a cell or organism into which it is inserted when it would not naturally occur in that particular cell or organism.

The term “Huanglongbing disease,” as used herein, is a disease of plants caused by microorganisms of the Candidatus genus Liberibacter, such as L. asiaticus, L. africanus, and L. americanus. This disease, for example, can be found in citrus plants, or other plants in the genus Rutaceae. Symptoms of Huanglongbing disease include one or more of yellow shoots and mottling of the plant leaves, occasionally with thickening of the leaves, reduced fruit size, fruit greening, premature dropping of fruit from the plant, low fruit soluble acid content, fruit with a bitter or salty taste, or death of the plant. The term “treating” or “treatment,” or its cognates, as used herein indicates any process or method which cures, diminishes, ameliorates, or slows the progress of the disease or disease symptoms. Thus, treatment includes reducing bacterial titer in plant tissues or appearance of disease symptoms relative to controls which have not undergone treatment.

Hypersensitive Response (or Reaction): The hypersensitive response (or sometimes referred to a hypersensitive reaction) (HR) is plant defense mechanism that protects a plant against infection by a plant pathogen. HR is a form of cell death often associated with plant resistance to pathogen infection to prevent the spread of the potential pathogen from infected to uninfected tissues. Cell death is activated by recognition of pathogen-derived molecules by the resistance (R) gene products, and is associated with the massive accumulation of reactive oxygen species (ROS), salicylic acid (SA), and other pro-death signals such as nitric oxide (NO). Ca. Liberibacter species inhibit hypersensitive response, which inhibits the plant from defending itself against the Ca. Liberibacter, Xanthomonas species, and other pathogens It is shown herein that secretion of SDEs by a bacterial species inhibit HR. The genomic modifications described herein prevent or minimize inhibition of HR by SDES.

The term “micronutrient” refers to nutrients that an organism needs for healthy growth and development. A non-limiting list of examples of micronutrients includes carbon, hydrogen, nitrogen, oxygen, phosphorus, potassium, sodium, calcium, and magnesium, as well as trace elements such as iron, sulfur, boron, chlorine, manganese, zinc, nickel, molybdenum, copper, iodine, selenium, and cobalt.

Overexpress: As used herein, “overexpress” or “overexpression” refers to increased expression of a gene or coding sequence over that found in nature or a control plant or tissue. In some embodiments, “overexpress” may refer to greater expression of a gene or coding sequence in a genetically modified plant, when compared to a plant lacking the genetic modification.

Plant: As used herein, the term “plant” refers to citrus or solanaceous plant, or any other plant that can be infected by a Ca Liberibacter species.

The terms “plant growth hormone” or “phytohormones” refer to organic substances that regulate plant growth and development. Plant growth hormones include auxins, gibberellins (GA), abscisic acid (ABA), cytokinins (CK), salicylic acid (SA), ethylene (ET), jasmonates (JA), brassinosteroids (BR), or peptides. Synthetic plant growth hormones or PGRs maybe used in place of a plant growth hormone.

The term “plant in need thereof,” as used herein, means any plant which is healthy or which has been diagnosed with a plant disease or symptoms thereof, or which is susceptible to a plant disease, or may be exposed to a plant disease or carrier thereof.

Plant part: The term “plant part” refer to cells, tissues, organs, seeds, and severed parts (e.g., roots, leaves, and flowers) that retain the distinguishing characteristics of the parent plant. “Seed” refers to any plant structure that is formed by continued differentiation of the ovule of the plant, following its normal maturation point at flower opening, irrespective of whether it is formed in the presence or absence of fertilization and irrespective of whether or not the seed structure is fertile or infertile. A plant part may be any part of the plant from which another plant may arise.

Promoter: A recognition site on a DNA sequence or group of DNA sequences that provides an expression control element for a structural gene and to which RNA polymerase specifically binds and initiates RNA synthesis (transcription) of that gene. Ro genetically modified plant: A plant that has been genetically modified or has been regenerated from a plant cell or cells that have been genetically modified. The terms “reactive oxygen species” or “ROS” refer to highly reactive chemicals formed from O2. Elevated formation of the different ROS leads to molecular damage, denoted as ‘oxidative distress’. Examples of ROS include peroxides, superoxide, hydroxyl radical, singlet oxygen, and alpha-oxygen.

Reduction of Expression: The term “Reduc(e), (es) or (ing) the expression” of a gene or polypeptide in a plant or a plant cell includes inhibiting, interrupting, knocking-out, or knocking-down the gene or polypeptide, such that transcription of the gene and/or translation of the encoded polypeptide is reduced as compared to a corresponding control plant, plant cell, or population of plants or plant cells in which expression of the gene or polypeptide is not inhibited, interrupted, knocked-out, or knocked-down. “Reduced expression” encompasses any decrease in expression level (e.g., a decrease of 10% or more, 20% or more, 30% or more, 40% or more, 50% or more, 60% or more, 70% or more, 80% or more, 90% or more, or even 100%) as compared to the corresponding control plant, plant cell, or population of plants or plant cells. In some embodiments, reducing expression by 50% or more may be particularly useful. Expression levels can be measured using methods such as, for example, reverse transcription-polymerase chain reaction (RT-PCR), Northern blotting, dot-blot hybridization, in situ hybridization, nuclear run-on and/or nuclear run-off, RNase protection, or immunological and enzymatic methods such as ELISA, radioimmunoassay, and western blotting

Regeneration: The process of growing a plant from a plant cell (e.g., plant protoplast, callus, or explant).

Rootstock: As used herein, a “rootstock” refers to underground plant parts such as roots, from which new above-ground growth of a plant or tree can be produced. In accordance with the disclosure, a rootstock may be used to grow a different variety through asexual propagation or reproduction such as grafting. As used herein, a “scion” refers to a plant part that is grafted onto a rootstock variety. A scion may be from the same or a different plant type or variety. [089] Site-specific genome modification: Any genome modification technique that employs an enzyme that can modify a nucleotide sequence in a sequence-specific manner. Site-specific genome modification enzymes include, but are not limited to, nucleases, endonucleases, recombinases, invertases, transposases, methytransferases, demethlylases, aminases, deaminases, helicases, and any combination thereof.

Transformation construct: A chimeric DNA molecule which is designed for introduction into a host cell by genetic transformation. Preferred transformation constructs will comprise all of the genetic elements necessary to direct the expression of one or more exogenous nucleic acid sequences. In particular embodiments of the instant disclosure, it may be desirable to introduce a transformation construct into a host cell in the form of an expression cassette.

Transgene: A segment of DNA which has been incorporated into a host genome or is capable of autonomous replication in a host cell and is capable of causing the expression of one or more nucleic acid sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was modified with the DNA segment.

Tolerance or resistance: Tolerance encompasses any relief from, reduced presentation of, improvement of, or any combination thereof of any symptom of an infection by a Ca. Liberibacter species. Resistance encompasses tolerance as well as a reduction of bacteria upon infection or reduction of ability to infect by a Ca. Liberibacter species. In specific embodiments of the disclosure, citrus plant may be provided that are defined as comprising a complete or less than complete resistance or tolerance to HLB. This may be assessed, for example, relative to a citrus plant not comprising a genetic modification according to the disclosure.

Vector: A DNA molecule designed for transformation into a host cell. Some vectors may be capable of replication in a host cell. A plasmid is an exemplary vector, as are expression cassettes isolated therefrom.

### DETAILED DESCRIPTION

### Overview

Citrus HLB is an immune-mediated disease. CLas induces a systemic chronic immune response, mimicking systemic chronic inflammation diseases of human³⁵. Systemic chronic inflammation diseases have been suggested to result from collateral damage to tissues and organs over time by oxidative stress³⁶. ROS concentrations triggered by CLas infection are above the threshold needed to induce cell death. Persistent induction of ROS by systemic CLas infection leads to systemic cell death of phloem tissues and other effects owing to diverse roles of ROS, which subsequently affect phloem function, hormone synthesis and transportation, metabolic transportation, and rerouting energy to immune response rather than to growth. This hypothesis can explain most HLB phenomena. For instance, phloem dysfunction resulting from death of companion and sieve element cells may lead to starch accumulation, and blotchy mottle symptoms. Hardened leaves perhaps result from the action of ROS since ROS are known to directly cause strengthening of host cell walls²⁵. Both cell death of the phloem tissues and reduced transportation of photosynthates may be responsible for root decay. Stunt growth probably results from the direct effect of ROS, reduced transportation of carbohydrates and hormones. The detailed molecular mechanism of how CLas activates immune response remains unknown. It is anticipated that cytoplasmic receptors, such as nucleotide-binding leucine-rich repeat (NLR) proteins are mainly responsible for intracellular detection of CLas through recognition of PAMPs inside companion and sieve element cells. It is probable that immune-mediated diseases, even though which have not been previously known for plants, are prevalent for the Plantae Kingdom, such as diseases caused by phloem-colonizing pathogens including bacteria (e.g., Ca. Liberibacter, Spiroplasma, and Ca. Phytoplasma), viruses, and fungi and some non-phloem colonizing pathogens.

Knowing citrus HLB is an immune mediated disease helps guide the battle against this notorious disease. It is projected that horticultural approaches that suppress oxidative stress can provide immediate help to alleviate the immune-mediated damages caused by CLas in HLB endemic citrus production areas. These approaches include optimized usage of plant growth hormones, such as GA and brassinosteroids³⁷. Even though the effect of nutritional modulation of immune function was not tested on HLB in this study, citrus growers in Florida have observed that modulation of macronutrients (N, P, and K) and micronutrients (e.g., B, Cu, Fe, Mn, Mo, Ni, Se and Zn) reduces HLB symptoms. This is consistent with that a deficiency of the macronutrients leads to oxidative stress³⁸, whereas micronutrients (B, Cu, Fe, Mn, Mo, Ni, Se and Zn) at low concentrations activate antioxidative enzymes 39. Growth hormones (e.g., GA) and nutritional modulation (e.g., micronutrients) directly alleviate the oxidative stress to reduce cell death of the phloem tissues to mitigate HLB symptoms. Moreover, growth hormones and micronutrients promote new growth, which decreases the ratio of dead cells in phloem tissues, further mitigating HLB symptoms. The horticultural measures used to mitigate ROS and cell death are expensive and unable to reduce or eliminate CLas inoculum, thus are not recommended for citrus production areas with low HLB incidence. For those areas, region-wide comprehensive implementation of roguing infected trees, tree replacement, and insecticide applications has been shown to successfully control citrus HLB^{6, 40}. In summary, citrus HLB is an immune-mediated disease and mitigating ROS via plant growth hormone mechanisms and promoting new growth both can reduce cell death of the phloem tissues, thus controlling HLB.

Compositions and formulations Preferably, the compounds are administered in the form of a composition containing a botanically compatible vehicle. Suitable amounts for administration to a plant are in the range of about 200 mL to about 500 mL for trunk injection, the range of about 1 per tree to about 4 L per tree for foliar spraying, and the range of about 1 gallon per to about 2 gallons per tree for soil drench and soil injection methods. Persons of skill in the art are able to adjust these amounts taking into account the plant size, timing of application and environmental conditions.

Compositions according to embodiments of the invention preferably include a botanically acceptable vehicle or carrier, preferably a liquid, aqueous vehicle or carrier such as water, and at least one compound according to the invention. The composition may be formulated as an emulsifiable concentrate(s), suspension concentrate(s), directly sprayable or dilutable solution(s), coatable paste(s), dilute emulsion(s), wettable powder(s), soluble powder(s), dispersible powder(s), dust(s), granule(s) or capsule(s).

The composition may optionally include a botanically acceptable carrier that contains or is blended with additional active ingredients and/or additional inert ingredients. Active ingredients which can be included in the carrier formulation can be selected from any combination of pesticides, herbicides, plant nutritional compositions such as fertilizers, and the like. Additional active ingredients can be administered simultaneously with the plant defense inducer compounds described here, in the same composition, or in separate compositions, or can be administered sequentially.

Inert ingredients which can be included in the carrier formulation can be selected from any compounds to aid in the physical or chemical properties of the composition. Such inert ingredients can be selected from buffers, salts, ions bulking agents, colorants, pigments, dyes, fillers, wetting agents, dispersants, emulsifiers, penetrants, preservatives, antifreezes, evaporation inhibitors, bacterial nutrient compounds, anti-caking agents, defoamers, antioxidants, and the like.

### Methods of Administration

Persons of skill are aware of various methods to apply compounds, including the compounds of the invention, to plants for surface application or for uptake, and any of these methods are contemplated for use in this invention. Methods of administration to plants include, by way of non-limiting example, application to any part of the plant, by inclusion in irrigation water, by injection into the plant or into the soil surrounding the plant, by exposure of the root system to aqueous solutions containing the compounds, by use in hydroponic or aeroponic systems, by culture of individual or groups of plant cells in media containing the inducer, by seed treatment, by exposure of cuttings of citrus plants used for grafting to aqueous solutions containing the compounds, by application to the roots, stems or leaves, or by application to the plant interior, or any part of the plant to be treated. Any means known to those of skill in the art is contemplated. Preferred modes of administration include those where the compounds are applied at, on or near the roots of the plant, or trunk injection.

Application of compounds can be performed in a nursery setting, a greenhouse, hydroponics facility, or in the field, or any setting where it is desirable to treat plants to prevent the likelihood of disease, or to treat disease and its effects, for example in plants which have been or can become exposed to HLB or Ca. Liberibacter infection. The methods and compounds of this invention can be used to treat infection with any Ca. Liberibacter species or type and can be used to improve plant defenses in plants which are not infected. Thus, any plant in need, in the context of this invention, includes any and all plants for which improvements in health and vigor, growth and productivity or ability to combat disease is desired. Citrus or other plants susceptible to diseases such as HLB or infection by Ca. Liberibacter species, whether currently infected or in potential danger of infection, in the judgement of the person of skill in this and related arts, are advantageously used in the invention.

Application to seeds preferably is accomplished as follows, however any method known in the art can be used. Seeds may be treated or dressed prior to planting, by soaking the seeds in a solution containing the compounds at a dosage of active ingredient over a period of minutes or hours, or by coating the seeds with a carrier containing the compounds at a dosage of active ingredient. The concentrations, volumes, and duration may change depending on the plant. Application to soil preferably is performed by soil injection or soil drenching, however any method known in the art can be used. These methods of administration are accomplished as follows. Soil drenching may be performed by pouring a solution or vehicle containing the compounds at a dosage of active ingredient at X to Y gallons/tree to the soil surface in a crescent within 10 to 100 cm of the trunk on the top side of the bed to minimize runoff, and/or by using the irrigation system. Soil injection may be performed by directly injecting a solution or vehicle containing the compounds at a dosage of active ingredient into the soil within 10 to 100 cm of the trunk using a soil injector. The concentrations, volumes, and duration may change depending on the plant.

Application to hydroponic or culture media preferably is performed as follows, however any method known in the art can be used. A solution or vehicle containing the compounds at a dosage of active ingredient may be added into the hydroponic or culture media at final concentrations suitable for plant growth and development. The concentrations, and volumes may change depending on the plant.

Application to the roots preferably is performed by immersing the root structure in a solution or vehicle in a laboratory, nursery or hydroponics environment, or by soil injection or soil drenching to the soil surrounding the roots, as described above. Emersion of the root structure preferably is performed as follows, however any method known in the art can be used. A solution or vehicle containing the compounds at a dosage of active ingredient may be applied to the roots by using a root feeder at 0.5 to 1 gallon per tree. The concentrations, volumes, and duration may change depending on the plant.

Application to the stems or leaves of the plant preferably is performed by spraying or other direct application to the desired area of the plant, however any method known in the art can be used. A solution or vehicle containing the compounds at a dosage of active ingredient may be applied with a sprayer to the stems or leaves until runoff to ensure complete coverage, and repeat three or four times in a growing season. The concentrations, volumes and repeat treatments may change depending on the plant.

Application to the plant interior preferably is performed by injection directly into the plant, for example by trunk injection or injection into an affected limb, however any method known in the art can be used.

### Genome Editing

Certain aspects of the present disclosure relate to methods of modifying the genome of a citrus or solanaceous plant using genome editing techniques. As used herein, “genome editing” and “genome-engineering” are terms used interchangeably and refer to the modification of a genome through mutagenesis. For example, in plant genome engineering, endonucleases may be used to generate double-strand DNA breaks (DSBs) and activate genome repair pathways. These DSB repair pathways may repair the break cleanly, i.e., without altering the starting sequence, or, alternatively, induce a mutation through an error in repair. In some embodiments, genome editing is used to insert, delete, or substitute one or more base pairs at one or any combination of genetic loci. In some embodiments, a genome editing technique is used to create a mutation, for example, a point mutation or single nucleotide polymorphism.

In some embodiments the DSB repair pathway is non-homologous end-joining (NHEJ) or microhomology mediated end joining (MMEJ). During NHEJ, any nucleotide overhangs on the break ends are either resected or filled to form blunt ends that are ligated. During MMEJ, the break ends are processed to reveal overhangs comprising microhomology sequences that are then ligated together. The insertions or deletions resulting from the terminal end processing in both the NHEJ and MMEJ pathways can be referred to as indels. In some embodiments, the NHEJ or MHEJ that occurs can be relied upon to introduce a genome modification including, but not limited to, a silent mutation, a neutral mutation, a missense mutation, a nonsense mutation, or a frameshift mutation.

In other embodiments, the DSB repair pathway is homologous recombination (HR). During HR, a DSB is repaired using a template with sequences with homology to the DNA flanking the break, i.e., a homologous chromosome. In plant genome editing, a linear DNA polynucleotide flanked by sequences (e.g., of 50 base pairs or more) homologous to those flanking a targeted genomic locus, may be introduced into the genome when a DSB is repaired by HR. In some embodiments, this approach is used to introduce, substitute, or delete a DNA sequence at a genomic locus. Any DNA sequence of interest may be introduced, deleted, or substituted. An introduced or substituted DNA sequence may encode an RNA molecule with a specific activity or function, a DNA molecule with a specific activity or function (e.g., encoding a polypeptide, representing a detectable marker, etc.), a DNA molecule comprising ds-regulatory elements, or a DNA molecule encoding a polypeptide, a motif thereof, or domain thereof. In some embodiments, the nucleic acid encoding the linear DNA sequence that will act as the HR template is encoded by an expression vector. In some embodiments, the nucleic acid encoding the linear DNA sequence of interest is encoded by a DNA sequence separate from the expression vector. For example, and without limitation, the nucleic acid encoding a DNA sequence of interest may be a linear DNA polynucleotide that is co-transformed with an expression vector.

In some embodiments, single-strand breaks or “nicks” are introduced into the target DNA sequence. As used herein, the term “single-strand break inducing agent” or “nickase” refers to any agent that can induce a single-strand break (SSB) in a DNA molecule. In some embodiments two SSBs are introduced into the target DNA to generate a DSB. These breaks may also be repaired by HR, NHEJ, or MMEJ. In some embodiments, sequence modifications occur at or near the SSB sites, which can include deletions or insertions that result in modification of the nucleic acid sequence, or integration of exogenous nucleic acids by HR or NHEJ.

In one aspect, a “modification” comprises the insertion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In another aspect, a “modification” comprises the deletion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In a further aspect, a “modification” comprises the inversion of at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the substitution of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In some embodiments, a “modification” comprises the substitution of an “A” for a “C,” “G” or “T” in a nucleic acid sequence. In some embodiments, a “modification” comprises the substitution of an “C” for an “A,” “G” or “T” in a nucleic acid sequence. In some embodiments, a “modification” comprises the substitution of a “G” for an “A,” “C” or “T” in a nucleic acid sequence. In some embodiments, a “modification” comprises the substitution of a “T” for an “A,” “C” or “G” in a nucleic acid sequence. In some embodiments, a “modification” comprises the substitution of a “C” for an “U” in a nucleic acid sequence. In some embodiments, a “modification” comprises the substitution of a “G” for an “A” in a nucleic acid sequence. In some embodiments, a “modification” comprises the substitution of an “A” for a “G” in a nucleic acid sequence. In some embodiments, a “modification” comprises the substitution of a “T” for a “C” in a nucleic acid sequence.

In some embodiments, genome editing of a citrus plant as described herein may encompass techniques that employ methods of targeting endonucleases to one or more genetic loci. In some embodiments, synthetic polypeptides, for example, Transcription Activator-Like Effectors (TALEs) and zinc fingers (ZFs), or nucleic acids, for example, Clustered Regularly Interspaced Short Palindromic Repeats/Cas (CRISPR/CAS) single guide RNAs or NgAgo (Argonaute) single strand DNAs, are used to target endonucleases to any genomic locus. The targeted endonucleases may catalyze a DSB at a target locus. Upon detecting these breaks, a cell may initiate any DSB repair pathway. In some embodiments, genome editing is carried out at more than one genomic locus simultaneously (i.e., multiplex genome engineering). In some embodiments, multiplex genome engineering may be used to remove a sequence of any size from the genome. In some embodiments, any combination and number of endonuclease targeting techniques may be used to target one or more genetic loci.

### RNA- and DNA-Guided Genome Editing Systems

In some embodiments, genome engineering of a citrus plant as described herein may employ RNA-guided endonucleases including, but not limited to CRISPR/Cas systems. CRISPR/Cas systems have been described in U.S. Patent Application Publication Nos. 2017/0191082 and 2017/0106025, each of which are incorporated herein by reference in their entirety. In some embodiments, a targeted genome modification as described herein comprises the use of at least one, at least two, at least three, at least four, at least five, at least six, at least seven, at least eight, at least nine, or at least ten RNA-guided nucleases. In some embodiments, a CRISPR/Cas9 system, a CRISPR/Cpf1 system, a CRISPR/CasX system, or a CRISPR/CasY system are alternatives that may be used to generate modifications to target sequences as described herein.

The CRISPR systems are based on RNA-guided endonucleases that use complementary base pairing to recognize DNA sequences at target sites. CRISPR/Cas systems are part of the adaptive immune system of bacteria and archaea, protecting them against invading DNA, such as viral DNA, by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trafts-activating CRISPR RNA (tracrRNA) to activate and guide the Cas9 nuclease. This cleaves homologous double-stranded DNA sequences known as protospacers in the invading DNA.

A prerequisite for cleavage is the presence of a conserved protospacer-adjacent motif (PAM) downstream of the target DNA, which usually has the sequence 5′-NGG-3′ but less frequently NAG. Specificity is provided by the so-called “seed sequence” approximately 12 bases upstream of the PAM, which must match between the RNA and target DNA. Cpf 1 acts in a similar manner to Cas9, but Cpf 1 does not require a tracrRNA. Specificity of the CRISPR/Cas system is based on an RNA-guide that use complementary base pairing to recognize target DNA sequences. In some embodiments, the site-specific genome modification enzyme is a CRISPR/Cas system. In an aspect, a site-specific genome modification enzyme provided herein can comprise any RNA-guided Cas endonuclease (non-limiting examples of RNA-guided nucleases include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, Cpf1, homologs thereof, or modified versions thereof); and, optionally, the guide RNA necessary for targeting the respective nucleases.

In some embodiments, an RNA-guided endonuclease is the DNA cleavage domain of a restriction enzyme fused to a deactivated Cas9 (dCas9), for example dCas9-Fok1. As used herein, a “dCas9” refers to a endonuclease protein with one or more amino acid mutations that result in a Cas9 protein without endonuclease activity, but retaining RNA-guided site-specific DNA binding. As used herein, a “dCas9-restriction enzyme fusion protein” is a dCas9 with a protein fused to the dCas9 in such a manner that the restriction enzyme is catalytically active on the DNA.

In some embodiments, genome editing of a citrus or solanaceous plant as described herein may employ DNA-guided endonucleases including, but not limited to, NgAgo systems.

In one aspect, a method and/or composition provided herein comprises one or more, two or more, three or more, four or more, or five or more guide RNAs or DNAs. In another aspect, a CRISPR/CAS system, dCas9-restriction enzyme fusion protein, NgAgo system provided herein is capable of generating a targeted DSB in a target sequence as described herein. In one aspect, vectors comprising nucleic acids encoding one or more, two or more, three or more, four or more, or five or more guide RNAs or DNAs and the corresponding CRISPR/CAS system, dCas9-restriction enzyme fusion protein, NgAgo system are provided to a cell by transformation methods known in the art (e.g., without being limiting, viral transfection, particle bombardment, PEG-mediated protoplast transfection or Agrobacterium-mediated transformation).

### Transcription Activator-Like Effector Nucleases

In some embodiments, genome editing of a citrus plant as described herein may employ Transcription Activator-Like Effector Nucleases (TALENs). TALENs have been described in U.S. Patent Application Publication Nos. 2016/0369301 and 2015/0203871 (both of which are incorporated herein by reference in their entirety) and are well known in the art. TALENs are artificial restriction enzymes generated by fusing the transcription activator-like effector (TALE) DNA binding domain to an endonuclease domain, hi one aspect, the nuclease is selected from a group consisting of Pvu11, MutU, Tev1 and Fok Alw1, Mly1, Sbf1, Sda1, Sts1, CleDORF, Clo051, Pept071. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site.

TALEs can be engineered to bind practically any DNA sequence, such as a target sequence as described herein. TALE proteins are DNA-binding domains derived from various plant bacterial pathogens of the genus Xanthomonas. The X pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

In one aspect, a method and/or composition provided herein comprises one or more, two or more, three or more, four or more, or five or more TALENs. In another aspect, a TALEN provided herein is capable of generating a targeted DSB in a target sequence as described herein. In one aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agwbacterium-ediaied transformation).

### Zinc Finger Nucleases

In some embodiments, genome engineering of a citrus or solanaceous plant as described herein may employ Zinc Finger Nucleases (ZFNs). ZFNs have been described in U.S. Pat. No. 9,322,006 (incorporated herein by reference in its entirety) and are well known in the art. ZFNs are synthetic proteins consisting of an engineered zinc finger DNA-binding domain fused to the cleavage domain of an endonuclease, for example, Fok1. ZFNs can be designed to cleave almost any long stretch of double-stranded DNA by the modification of the zinc finger DNA-binding domain. ZFNs form dimers from monomers composed of a non-specific DNA cleavage domain of Fok1 nuclease fused to a zinc finger array engineered to bind a target DNA sequence. The DNA-binding domain of a ZFN is typically composed of 3-4 zinc-finger arrays. The amino acids at positions −1, +2, +3, and +6 relative to the start of the zinc finger∞-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art. The Fok1 nuclease domain requires dimerization to cleave DNA and therefore two ZFNs with their C-terminal regions are needed to bind opposite DNA strands of the cleavage site (separated by 5-7 nt). The ZFN monomer can cut the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Without being limited by any scientific theory, because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

Several embodiments relate to a method and/or composition provided herein comprising one or more, two or more, three or more, four or more, or five or more ZFNs directed to a target sequence as described herein. In another aspect, a ZFN provided herein is capable of generating a targeted DSB. In one aspect, vectors comprising polynucleotides encoding one or more, two or more, three or more, four or more, or five or more ZFNs are provided to a cell by transformation methods known in the art (e.g., without being limiting, viral transfection, particle bombardment, PEG-mediated protoplast transfection or Agrobacterium-mediated transformation).

### Meganucleases

In some embodiments, genome engineering of a citrus or solanaceous plant as described herein may employ a meganuclease. Meganucleases, which are commonly identified in microbes, are unique enzymes with high activity and long recognition sequences (>14 nt) resulting in site-specific digestion of target DNA. Engineered versions of naturally occurring meganucleases typically have extended DNA recognition sequences (for example, 14 to 40 nt). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

In one aspect, a method and/or composition provided herein comprises one or more, two or more, three or more, four or more, or five or more meganucleases directed to a target sequence as described herein. In some embodiments, a meganuclease provided herein is capable of generating a targeted DSB. In some embodiments, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation).

### Site-Specific Genome Modification

Certain aspects of the present disclosure relate to methods of modifying the genome of a citrus plant using site-specific genome modification techniques. In some embodiments, site-specific genome modification of a citrus plant as described herein may employ any site-specific genome modification enzyme. As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme is a recombinase. In some embodiments, a site-specific genome modification enzyme is a transposase. In the present disclosure, site-specific genome modification enzymes include, but are not limited to, nucleases, endonucleases, recombinases, invertases, transposases, methytransferase, demethlylases, aminases, deaminases, helicases, and any combination thereof.

In some embodiments, the site-specific genome modification enzyme is a recombinase. Non-limiting examples of recombinases include a tyrosine and serine recombinases and coupled with a DNA recognition motifs, for example, a Cre recombinase, a Gin recombinase, a Flp recombinase, and a Tnp1 recombinase. In another aspect, a serine recombinase coupled with a DNA recognition motif, for example, a PhiC31 integrase, an R4 integrase, and a TP-901 integrase. In an aspect, a recombinase is tethered to a zinc-finger DNA-binding domain, or a TALE DNA-binding domain, or a Cas9 nuclease. [0130] The Flp-FRT site-directed recombination system comes from the 2 plasmid from the baker's yeast Saccharomyces cerevisiae. In this system, Flp recombinase (flippase) recombines sequences between flippase recognition target (FRT) sites. FRT sites comprise 34 nucleotides. Flp binds to the “arms” of the FRT sites (one arm is in reverse orientation) and cleaves the FRT site at either end of an intervening nucleic acid sequence. After cleavage, Flp recombines nucleic acid sequences between two FRT sites.

Cre-lox is a site-directed recombination system derived from the bacteriophage PI that is similar to the Flp-FRT recombination system. Cre-lox can be used to invert a nucleic acid sequence, delete a nucleic acid sequence, or translocate a nucleic acid sequence. In this system, Cre recombinase recombines a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or Mi 1 site.

In another aspect, the site-specific genome modification enzyme is a dCas9-recombinase fusion protein. As used herein, a “dCas9-recombinase fusion protein” is a dCas9 with a protein fused to the dCas9 in such a manner that the recombinase is catalytically active on the DNA. In some embodiments, dCas9 may be fused with the catalytic domain of any enzyme such that the catalytic domain is catalytically active on DNA. In another aspect, a DNA transposase is attached to a DNA binding domain for example, a TALE-piggyBac and TALE-Mutator.

Several embodiments relate to promoting DNA recombination by providing a site-specific genome modification enzyme to a plant cell. In some embodiments, recombination is promoted by providing a strand separation inducing reagent. In one aspect, the site-specific genome modification enzyme is selected from an endonuclease, a recombinase, an invertase, a transposase, a helicase or any combination thereof. In some embodiments, recombination occurs between B chromosomes. In some embodiments, recombination occurs between a B chromosome and an A chromosome.

Several embodiments relate to promoting integration of one or more DNAs of interest by providing a site-specific genome modification enzyme. In some embodiments, integration of one or more DNAs of interest is promoted by providing a strand separation inducing reagent. In one aspect, the site-specific genome modification enzyme is selected from an endonuclease, a recombinase, a transposase, a helicase or any combination thereof. Any DNA sequence can be integrated into a target site of a chromosome sequence by introducing the DNA sequence and the provided site-specific genome modification enzymes. Any method provided herein can utilize any site-specific genome modification enzyme provided herein.

Several embodiments relate to a method and/or a composition provided herein comprising at least one, at least two, at least three, at least four, at least five, at least six, at least seven, at least eight, at least nine, or at least ten site-specific genome modification enzymes. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific genome modification enzymes.

Plant Transformation Constructs Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. In some embodiments, a viral vector based on a plant virus such as a Citrus Tristeza Virus may be used in accordance with the disclosure, namely for delivery of vectors to plant cells. It is contemplated that utilization of cloning systems with large insert capacities will allow introduction of large genetic sequences comprising more than one selected gene. In accordance with the disclosure, this could be used to introduce genetic material corresponding to an entire biosynthetic pathway into a plant. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes that have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant genetically modified cells resulting in a screenable or selectable trait and/or will impart an improved phenotype to the resulting genetically modified plant. However, this may not always be the case, and the present disclosure also encompasses genetically modified plants incorporating non-expressed transgenes.

In accordance with the disclosure, a nucleic acid vector comprising a coding sequence may be introduced into a plant such as a citrus tree or variety, such that, when the vector is transformed into a citrus variety or plant as described herein, the coding sequence is expressed in the plant. In some embodiments the coding sequence may be expressed in, for example, the phloem or roots of the plant, or any other part of the plant. Expression of the coding sequence in the resulting genetically modified citrus tree or variety results in the tree exhibiting increased tolerance or resistance to HLB when compared to a tree lacking expression of the coding sequence.

### Proteins and Recombinant DNA Molecules

As used herein, a “protein/Coding DNA molecule” or “polypeptide/Coding DNA molecule” refers to a DNA molecule comprising a nucleotide sequence that encodes a protein or polypeptide. A “coding sequence” or “protein/Coding sequence” or “polypeptide/Coding sequence” means a DNA sequence that encodes a protein or polypeptide. A “sequence” means a sequential arrangement of nucleotides or amino acids. The boundaries of a protein/Coding sequence or polypeptide/Coding sequence are usually determined by a translation start codon at the 5′-terminus and a translation stop codon at the 3′-terminus. A protein/Coding molecule or polypeptide/Coding molecule may comprise a DNA sequence encoding a protein or polypeptide sequence. As used herein, “transgene expression,” “expressing a transgene,” “protein expression,” “polypeptide expression,” “expressing a protein,” and “expressing a polypeptide” mean the production of a protein or polypeptide through the process of transcribing a DNA molecule into messenger RNA (mRNA) and translating the mRNA into polypeptide chains, which may be ultimately folded into proteins. A protein/Coding DNA molecule or polypeptide/Coding DNA molecule may be operably linked to a heterologous promoter in a DNA construct for use in expressing the protein or polypeptide in a cell transformed with the recombinant DNA molecule. As used herein, “operably linked” means two DNA molecules linked in manner so that one may affect the function of the other. Operably-linked DNA molecules may be part of a single contiguous molecule and may or may not be adjacent. For example, a promoter is operably linked with a protein/Coding DNA molecule or polypeptide/Coding DNA molecule in a DNA construct where the two DNA molecules are so arranged that the promoter may affect the expression of the transgene.

As used herein, a “DNA construct” is a recombinant DNA molecule comprising two or more heterologous DNA sequences. DNA constructs are useful for transgene expression and may be comprised in vectors and plasmids. DNA constructs may be used in vectors for the purpose of genome modification, that is the introduction of heterologous DNA into a host cell, in order to produce genetically modified plants and cells, and as such may also be contained in the plastid DNA or genomic DNA of a genetically modified plant, seed, cell, or plant part. As used herein, a “vector” means any recombinant DNA molecule that may be used for the purpose of genetically modifying a plant or plant cell. Recombinant DNA molecules as set forth in the sequence listing, can, for example, be inserted into a vector as part of a construct having the recombinant DNA molecule operably linked to a promoter that functions in a plant to drive expression of the protein encoded by the recombinant DNA molecule. Methods for constructing DNA constructs and vectors are well known in the art. The components for a DNA construct, or a vector comprising a DNA construct, generally include, but are not limited to, one or more of the following: a suitable promoter for the expression of an operably linked DNA, an operably linked protein/Coding DNA molecule, and a 3′ untranslated region (3′-UTR). Promoters useful in practicing the present disclosure include those that function in a plant for expression of an operably linked polynucleotide. Such promoters are varied and well known in the art and include those that are inducible, viral, synthetic, constitutive, temporally regulated, spatially regulated, and/or spatio-temporally regulated. Additional optional components include, but are not limited to, one or more of the following elements: 5′-UTR, enhancer, leader, cis-acting element, intron, chloroplast transit peptides (CTP), and one or more selectable marker transgenes.

Recombinant DNA molecules of the present disclosure may be synthesized and modified by methods known in the art, either completely or in part, especially where it is desirable to provide sequences useful for DNA manipulation (such as restriction enzyme recognition sites or recombination-based cloning sites), plant-preferred sequences (such as plant/Codon usage or Kozak consensus sequences), or sequences useful for DNA construct design (such as spacer or linker sequences). The present disclosure includes recombinant DNA molecules and proteins having at least about 80% (percent) sequence identity, about 81% sequence identity, about 82% sequence identity, about 83% sequence identity, about 84% sequence identity, about 85% sequence identity, about 86% sequence identity, about 87% sequence identity, about 88% sequence identity, about 89% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, about 99% sequence identity, or about 100% sequence identity to a coding sequence provided herein. As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar, Inc., Madison, WI), and MUSCLE (version 3.6) (Edgar, Nucl. Acids Res. 32: 1792-1797, 2004) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

Proteins in accordance with the disclosure may be produced by changing (that is, modifying) a wild-type protein to produce a new protein with a novel combination of useful protein characteristics, such as altered Vmax, Km, substrate specificity, substrate selectivity, and protein stability. Modifications may be made at specific amino acid positions in a protein and may be a substitution of the amino acid found at that position in nature (that is, in the wild-type protein) with a different amino acid. Proteins provided by the disclosure thus provide a new protein with one or more altered protein characteristics relative to the wild-type protein found in nature. In one embodiment of the disclosure, a protein may have altered protein characteristics such as improved or decreased activity against one or more herbicides or improved protein stability as compared to a similar wild-type protein, or any combination of such characteristics.

In one embodiment, the disclosure provides a protein, and the DNA molecule or coding sequence encoding it, having at least about 80% sequence identity, about 81% sequence identity, about 82% sequence identity, about 83% sequence identity, about 84% sequence identity, about 85% sequence identity, about 86% sequence identity, about 87% sequence identity, about 88% sequence identity, about 89% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, about 99% sequence identity, or about 100% sequence identity to a protein sequence. Amino acid mutations may be made as a single amino acid substitution in the protein or in combination with one or more other mutation(s), such as one or more other amino acid substitution(s), deletions, or additions. Mutations may be made as described herein or by any other method known to those of skill in the art.

### Regulatory Elements

The plants and methods of the present disclosure can utilize a vector comprising a coding sequence that, when the vector is transfected into a plant, the coding sequence is expressed in the plant. The site and conditions under which the first selected DNA is expressed can be controlled to a great extent by selecting a promoter element in the vector that causes expression under the desired conditions.

In some embodiments, the coding sequence is expressed primarily in the roots of the plant, or in the phloem tissue of the plant. In this case, the coding sequence may be expressed in a greater quantity in roots or phloem than in other tissues of the plant. In some embodiments, more than one copy of an coding sequence may be expressed in a plant such that expression in the roots or phloem may be at least twice as much as in any other individual plant tissue (e.g., leaves, flowers, etc).

Limiting expression of the coding sequence primarily to the roots or phloem of a plant may be accomplished by operably linking the coding sequence to a heterologous promoter active in plant tissues, such as a root-specific or phloem-specific promoter. In other embodiments, a constitutive promoter may be preferred such that the coding sequence is expressed in all tissues of the plant. In some embodiments, a phloem-specific promoter in accordance with the disclosure may comprise an Arabidopsis sucrose-proton symporter 2 (AtSUC2) promoter, or a constitutive promoter may comprise a CaMV 35S promoter. Any root-specific or phloem-specific promoter known in the art may potentially be utilized to direct expression of the coding sequence to the roots or the phloem tissue. Examples of these may include, but are not limited to, an RB7, RPE15, RPE14, RPE19, RPE29, RPE60, RPE2, RPE39, RPE61, SHR, ELG3, EXP7, EXP 18 or Aflg73160 promoter (Vijaybhaskar et at, 2008; Kurata et at, 2005; PCT Publication WO 01/53502; U.S. Pat. No. 5,459,252; Cho and Cosgrove, 2002).

In some embodiments, a coding sequence as described herein may be expressed at any level in the plant such that it may be detected in the plant using techniques known in the art. A coding sequence may be expressed in a greater quantity in a genetically modified citrus plant or variety than in a plant not expressing the coding sequence as described herein. In some embodiments, the coding sequence is expressed at least twice as much as in a plant not expressing a coding sequence. In further embodiments, the coding sequence is expressed at least three, or four, or five times, or more, as much as in a plant not expressing a coding sequence. In yet another embodiment, there is no detectable expression of the coding sequence in a plant not expressing a coding sequence.

The DNA sequence between the transcription initiation site and the start of the coding sequence, i.e., the untranslated leader sequence, can also influence gene expression. One may thus wish to employ a particular leader sequence with a transformation construct of the disclosure. Useful leader sequences are contemplated to include those which comprise sequences predicted to direct optimum expression of the attached gene, i.e., to include a consensus leader sequence which may increase or maintain mRNA stability and prevent inappropriate initiation of translation. The choice of such sequences will be known to those of skill in the art in light of the present disclosure.

It is contemplated that vectors for use in accordance with the present disclosure may be constructed to include an ocs enhancer element. This element was first identified as a 16-bp palindromic enhancer from the octopine synthase (ocs) gene of Agrobacterium (Ellis et al, 1987), and is present in at least 10 other promoters (Bouchez et al, 1989). The use of an enhancer element, such as the ocs element and particularly multiple copies of the element, may act to increase the level of transcription from adjacent promoters when applied in the context of plant transformation.

### Terminators

Transformation constructs prepared in accordance with the disclosure will typically include a 3′ end DNA sequence that acts as a signal to terminate transcription and allow for the poly-adenylation of the mRNA produced by coding sequences operably linked to a promoter. In one embodiment of the disclosure, the native terminator of a coding sequence coding sequence may be used. Alternatively, a heterologous 3′ end may enhance the expression of coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et ah, 1987), sucrose synthase intron (Vasil et al, 1989) or TMV omega element (Gallie et al, 1989), may further be included where desired.

### Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, Golgi apparatus, and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a genetically modified plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

### Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Many examples of suitable marker proteins are known to the art and can be employed in the practice of the disclosure. Examples include, but not limited to, neo (Potrykus et al, 1985), bar (Hinchee et al, 1988), bxn (Stalker et al, 1988); a mutant acetolactate synthase (ALS) (European Patent Application 154, 204, 1985) a methotrexate resistant DHFR (Thillet et al, 1988), 0-glucuronidase (GUS); R-locus (Dellaporta et al, 1988), P-lactamase (Sutcliffe, 1978), xylE (Zukowsky et al., 1983), cc-amylase (Ikuta et al., 1990), tyrosinase (atz et al, 1983), 0-galactosidase, luciferase (lux) (Ow et al, 1986), aequorin (Prasher et al, 1985), and green fluorescent protein (Sheen et al, 1995; Haseloff et al, 1997; Reichel et al, 1996; Tian et al, 1997; WO 97/41228).

Included within the terms “selectable” or “screenable” markers also are genes which encode a “secretable marker” whose secretion can be detected as a means of identifying or selecting for genetically modified cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., a-amylase, P-lactamase, phosphinothricin acetyl transferase); and proteins that are inserted or trapped in the cell wall (e.g., proteins that include a leader sequence such as that found in the expression unit of extensin or tobacco PR-S).

### Antisense and RNAi Constructs

In the methods and compositions of the present disclosure, endogenous gene activity can be down-regulated by any means known in the art, including through the use of ribozymes or aptamers. Endogenous gene activity can also be down-regulated with an antisense or RNAi molecule.

In particular, constructs comprising a coding sequence, including fragments thereof, in antisense orientation, or combinations of sense and antisense orientation, may be used to decrease or effectively eliminate the expression of the gene in a plant such as a citrus tree or variety. Accordingly, this may be used to “knock-out” the function of the coding sequence or homologous sequences thereof.

Techniques for RNAi are well known in the art and are described in, for example, Lehner et al, (2004) and Downward (2004). The technique is based on the ability of double stranded RNA to direct the degradation of messenger RNA with sequence complementary to one or the other strand (Fire et al, 1998). Therefore, by expression of a particular coding sequence in sense and antisense orientation, either as a fragment or longer portion of the corresponding coding sequence, the expression of that coding sequence can be down-regulated.

Antisense, and in some aspects RNAi, methodology takes advantage of the fact that nucleic acids tend to pair with “complementary” sequences. By complementary, it is meant that polynucleotides are those which are capable of base-pairing according to the standard Watson/Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNA's, may be employed to inhibit gene transcription or translation or both within a host cell, either in vitro or in vivo, such as within a host plant cell. In certain embodiments of the disclosure, such an oligonucleotide may comprise any unique portion of a nucleic acid sequence provided herein. In certain embodiments of the disclosure, such a sequence comprises at least 18, 30, 50, 75, or 100 or more contiguous nucleic acids of the nucleic acid sequence of a gene, and/or complements thereof, which may be in sense and/or antisense orientation. By including sequences in both sense and antisense orientation, increased suppression of the corresponding coding sequence may be achieved. [0159] Constructs may be designed that are complementary to all or part of the promoter and other control regions, exons, introns or even exon-intron boundaries of a gene. It is contemplated that the most effective constructs may include regions complementary to intron/exon splice junctions. Thus, it is proposed that an embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an RNAi or antisense construct which has limited regions of high homology, but also contains a non-homologous region (e.g., as in a ribozyme) could be designed. Methods for selection and design of sequences that generate RNAi are well known in the art (e.g. Reynolds, 2004). These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence. Constructs useful for generating RNAi may also comprise concatemers of sub-sequences that display gene regulating activity.

### Methods for Genetic Transformation/Transfection

Suitable methods for transformation of plant or other cells for use with the current disclosure are believed to include virtually any method by which DNA can be introduced into a cell, such as by direct delivery of DNA such as by PEG-mediated transformation of protoplasts (Omirulleh et ai, 1993), by desiccation/inhibition-mediated DNA uptake (Potrykus et al, 1985), by electroporation (U.S. Pat. No. 5,384,253, specifically incorporated herein by reference in its entirety), by Agrobacterium-mediated transformation (U.S. Pat. Nos. 5,591,616 and 5,563,055; both specifically incorporated herein by reference) and by acceleration of DNA coated particles (U.S. Pat. Nos. 5,550,318; 5,538,877; and 5,538,880; each specifically incorporated herein by reference in its entirety), etc. Through the application of techniques such as these, the cells of virtually any plant species may be stably transformed, and these cells developed into genetically modified plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et ai, (1985), Rogers et ai, (1987) and U.S. Pat. No. 5,563,055, specifically incorporated herein by reference in its entirety.

Another method for delivering transforming DNA segments to plant cells in accordance with the disclosure is microprojectile bombardment (U.S. Pat. Nos. 5,550,318; 5,538,880; 5,610,042; and PCT Application WO 94/09699; each of which is specifically incorporated herein by reference in its entirety). In this method, particles may be coated with nucleic acids and delivered into cells by a propelling force.

Another method of delivering genetic information to plant cells (i.e. citrus cells) is via a Citrus Tristeza Virus vector. See U.S. Pat. Nos. 10,851,381; 10,781,454; 10,472,641; 10,093,939; and 9,611,483, which are incorporated herein by reference.

### Production and Characterization of Genetically Modified Plants

After effecting delivery of exogenous DNA to recipient cells, the next steps generally concern first identifying and selecting the transformed cells and from those cells identifying the selecting the genetically modified cells for further culturing and plant regeneration. In order to improve the ability to identify transformed and genetically modified cells, one may desire to employ one or more selectable or screenable marker genes with a transformation vector prepared in accordance with the disclosure. In this case, one would then generally assay the potentially transformed and modified cell population by exposing the cells to a selective agent or agents, or one would screen the cells for the desired marker gene trait.

It is believed that DNA is introduced into only a small percentage of target cells in any one study. In order to provide an efficient system for identification of those cells that are transformed and predisposed to genetic modification one may employ a means for selecting those cells that are stably transformed. One exemplary embodiment of such a method is to introduce, into the host cell, a marker gene which confers resistance to some normally inhibitory agent, such as an antibiotic or herbicide. Potentially transformed cells then are exposed to the selective agent. In the population of surviving cells will be those cells where, generally, the resistance/Conferring gene has been integrated and expressed at sufficient levels to permit cell survival. Cells may be tested further to confirm stable integration of the exogenous DNA.

Cells that survive the exposure to the selective agent, or cells that have been scored positive in a screening assay, may then be selected again using a second, distinct selection paradigm that detects those cells that contain the genetic modification. Cells that survive the exposure to the second selective agent, or cells that have been scored positive in the second screening assay, may be cultured in media that supports regeneration of plants. The genetically modified cells, identified by selection or screening and cultured in an appropriate medium that supports regeneration, will then be allowed to mature into plants. Developing plantlets are transferred to soilless plant growth mix, and hardened, e.g., in an environmentally controlled chamber, for example, at about 85% relative humidity, 600 ppm C0₂, and 25-250 microeinsteins m″² s″¹ of light. Plants may be matured in a growth chamber or greenhouse. Plants can be regenerated from about 6 wk to 10 months after a genetically modified cell is identified, depending on the initial tissue.

To confirm the presence of the genetic modification in the regenerating plants, a variety of assays may be performed. Such assays include, for example, “molecular biological” assays, such as Southern and northern blotting and polymerase chain reaction (PCR); “biochemical” assays, such as detecting the absence or presence of a protein product, e.g., by immunological means (ELISAs and western blots) or by enzymatic function; plant part assays, such as leaf or root assays; and also, by analyzing the phenotype of the whole regenerated plant. Modification of the host genome and the independent identities of genetically modified plants may be determined using, e.g., Southern hybridization or PCR. Genetic modifications that affect, for example, protein or gene expression may then be evaluated by specifically measuring the expression of those affected molecules or evaluating the phenotypic changes brought about by their expression change.

### Breeding Plants of the Disclosure

In addition to direct transformation of a particular plant genotype with a construct prepared according to the current disclosure, genetically modified plants may be made by crossing a plant having a selected genetic modification of the disclosure to a second plant lacking the construct. For example, a selected lignin biosynthesis coding sequence can be introduced into a particular plant variety by crossing, without the need for ever directly transforming a plant of that given variety. Therefore, the current disclosure not only encompasses a plant directly modified or regenerated from cells which have been modified in accordance with the current disclosure, but also the progeny of such plants.

As used herein the term “progeny” denotes the offspring of any generation of a parent plant prepared in accordance with the instant disclosure, wherein the progeny comprises a selected DNA construct. “Crossing” a plant to provide a plant line having one or more added transgenes relative to a starting plant line, as disclosed herein, is defined as the techniques that result in a coding sequence of the disclosure being introduced into a plant line by crossing a starting line with a donor plant line that comprises a first selected DNA of the disclosure. To achieve this in a plant such as a citrus tree one could, for example, perform the following steps:
(a) plant seeds of the first (starting line) and second (donor plant line that comprises a first selected DNA of the disclosure) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.

Backcrossing is herein defined as the process including the steps of:
(a) crossing a plant of a first genotype containing a desired gene, DNA sequence or element to a plant of a second genotype lacking the desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

In some embodiments, asexual reproduction or propagation may be used to obtain a progeny plant in accordance with the disclosure. Techniques to achieve asexual propagation or reproduction in citrus trees or varieties may include, for example, grafting, budding, top-working, layering, runner division, cuttings, rooting, T-budding, and the like. In some embodiments, one citrus variety into which a coding sequence has been introduced may be grafted onto the rootstock of another variety. In other embodiments, a coding sequence may be introduced into the rootstock. In either of these situations, one or both of the plant varieties may exhibit increased tolerance or resistance to HLB.

The percent sequence identity between a particular nucleic acid or amino acid sequence and a sequence referenced by a particular sequence identification number may be determined by techniques known in the art. In one example, sequence identity is determined as follows. First, a nucleic acid or amino acid sequence is compared to the sequence set forth in a particular sequence identification number using the BLAST 2 Sequences (B12seq) program from the standalone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12 seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q −1 -r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq2.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. For example, a nucleic acid sequence that has 1200 matches when aligned with a sequence having 1424 nucleotides is 83.7 percent identical to the sequence (i.e., 1200+1434 x100=83.7). It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It also is noted that the length value will always be an integer.

The embodiments described herein are not limited to a particular citrus or solanaceous plant or variety but rather encompass any citrus or solanaceous plant or hybrid thereof that may be useful in accordance with the disclosure. Citrus varieties contemplated by this disclosure include, but are not limited to, cultivated citrus types such as sweet orange, bitter orange, blood orange, grapefruit, pomelo, citron, Clementine, naval orange, lemon, lime, mandarin, tangerine, tangelo, or the like.

### EXAMPLES

### Methods and Materials

Transgenic expression analysis of CLas proteins containing Sec secretion signals and other putative virulence factors. Transgenic expression of CLas genes was conducted as described previously 1. For the citrus transformation, CLas genes were amplified without signal peptide sequence and cloned into the binary vector RCsVMV-erGFP-pCAMBIA-1380N-35S-BXKES-3×HA, which has a C-terminal 3×HA tag, to generate the CLas gene overexpression vectors. The resulting binary vectors were transferred into Agrobacterium tumefaciens strain EHA105 for citrus transformation. The empty vector (EV) was used in citrus transformation as a negative control. Agrobacterium-mediated transformation of epicotyl segments of Duncan grapefruit (Citrus paradisi) was carried out as described previously². Transgenic lines showing kanamycin-resistance and erGFP-specific fluorescence were selected and then micro-grafted in vitro onto one-month-old Carrizo citrange rootstock seedlings. After one month of growth in vitro, the grafted shoots were potted into a peat-based commercial potting medium and acclimated under greenhouse conditions for the phenotype evaluation. Transgenic plants were confirmed by PCR, qRT-PCR at the RNA level, or western blot using HA Tag Antibodies (Sigma-Aldrich, St. Louis, MO).

For the tobacco transformation, Agrobacterium-mediated transformation of leaf discs of Nicotiana tabacum was carried out to generate the transgenic tobacco 3. A. tumefaciens strain EHA105 containing the vectors was used for transformation. Transgenic positive shoots showing kanamycin-resistance and erGFP-specific fluorescence were selected and transferred to the rooting medium. Evaluation of the transgenic N. tabacum was conducted in a growth chamber. Transgenic plants were confirmed by PCR, qRT-PCR at the RNA level, or western blot using HA Tag Antibodies (Sigma-Aldrich).

For the gene overexpression in Arabidopsis thaliana. CLas genes without signal peptides were PCR amplified and cloned into the binary vector pCambia1380-35S-EYFP, which has a C-terminal EYFP protein tag, and transferred into A. tumefaciens strain GV2260. Agrobacterium-mediated floral dip method was used for the Arabidopsis transformation as reported previously 4. The T1 generation transgenic plants were screened on the Hygromycin B selection medium. Positive plants were further confirmed by PCR and western blot using GFP antibodies (Sigma-Aldrich). The positive T2 generation transgenic plants were evaluated in a growth chamber.

Plant materials used for investigation of the relationship between CLas infection, immune response, phloem blockage, cell death and HLB symptom development. Two-year-old CLas-infected and healthy Valencia sweet orange (Citrus sinensis) plants were used and maintained in a greenhouse (28° C.±2° C., relative humidity of 50%±5%, natural light period). Young flushes were selected from the twigs of HLB-positive sweet orange trees. Both ‘Valencia’ and ‘Hamlin’ sweet orange are C. sinensis and susceptible to HLB without observable differences in symptoms. The plant was infected with CLas by graft inoculation and maintained in a greenhouse. The sweet orange trees from the groves were naturally infected by CLas. Healthy plants were maintained in a glasshouse with natural light and without temperature control.

Quantification of H₂O₂ concentrations. H₂O₂ concentrations were quantified following the procedure described elsewhere²³. CLas positive asymptomatic mature leaves, mature leaves with mild or severe symptoms were collected from HLB-positive C. sinensis ‘Valencia’ trees in citrus groves of Citrus Research and Education Center, University of Florida/Institute of Food and Agricultural Sciences. CLas negative mature leaves were collected from healthy C. sinensis ‘Valencia’ trees in glasshouse. Briefly, leaf samples (0.5 g) were grinded in 0.1% (w/v) trichloroacetic acid (TCA) and centrifuged at 12,000 g for 15 min at 4° C. The supernatant (0.3 ml) was mixed with 1.7 ml 1.0 M potassium phosphate buffer (pH 7.0) and 1.0 ml of 1.0 M potassium iodide solution, then incubated for 5 min before measuring the absorbance of the oxidation product at 390 nm. H₂O₂ concentrations were calculated using a standard curve prepared with known concentrations of H₂O₂ and expressed in mol/g fresh weight. For measuring H₂O₂ concentrations in the exudates of phloem enriched bark tissues, the same procedure was used except the TCA step and H₂O₂ concentrations were expressed in mmol/L. CLas positive symptomatic branches and CLas-negative branches from the spring flush were used for collection of bark tissues from C. sinensis ‘Valencia’ trees mentioned above.

Ion leakage. Conductivity of the exudates extracted from phloem enriched bark tissues was measured using a CON 700 conductivity/° C./° F. bench meter (OAKTON Instruments, Vernon Hills, IL, USA).

Callose deposition assay. Leaf samples were fixed with FAA (37% formaldehyde/glacial acetic acid/95% ethanol/deionized water at a volume ratio of 50:5:10:35) solution overnight. Samples were embedded in the Tissue Plus O.C.T compound (Thermo-Fisher, Waltham, MA, USA), sectioned with a Harris Cryostat Microtome (International Equipment, Boston, MA, USA) and stained with 0.005% aniline blue solution prior to analysis. Samples were observed in an Olympus BX61 epifluorescence microscope (Olympus Corporation, Center Valley, PA, USA). Callose spots were counted per slide area for all sample types.

CLas quantification using qPCR. Tissues (100 mg) were homogenized into powders using a TissueLyser II (Qiagen, Valencia, CA, USA). DNA was extracted using the DNeasy Plant kit (Qiagen), following the manufacturer's instructions, and eluted in 100 μL nuclease free water. DNA concentration was measured using a Synergy LX plate reader (BioTek, Winooski, VT, USA). Quantification of CLas in plant tissues was performed as described elsewhere⁶. Briefly, qPCR was carried out with primers and probe for CLas⁷. qPCR assays were performed with QuantiStudio3 (Thermo Fisher, Waltham, MA) using the Quantitec Probe PCR Master Mix (Qiagen) in a 25-1 reaction. The standard amplification protocol was 95° C. for 10 min followed by 40 cycles at 95° C. for 15 s and 60° C. for 60 s. All reactions were conducted in triplicate with CLas positive and water controls. Quantification of CLas was conducted using the equation Y=−0.288×(CLas Ct)+11.607⁸.

Starch assay. The samples (100 mg) were powdered using a TissueLyser II (Qiagen, Hilden, Germany). The powdered samples were used to quantify the starch. The starch estimation was performed using the Total Starch Assay Kit (AA/AMG) (Megazyme, Bray, Ireland) as instructed by the manufacturer. The experiments were repeated thrice with similar result.

Statistical analyses. All statistical analyses were performed using SAS statistical software (Version 9.4, SAS Institute, Cary, NC, USA).

Gene expression assays using reverse transcription quantitative PCR (RT-qPCR).

Total RNA was extracted using the RNeasy Plant Mini Kit (Qiagen), according to manufacturer's instructions. cDNA was synthesized with Quantitec Reverse Transcription Kit (Qiagen) according to manufacturer's instructions and diluted 10 times for RT-qPCR. Reactions were carried out by adding 1 μL of cDNA, 1 μL of each specific primer, 7 μL water and 10 μL Fast SYBR Green Master Mix (Thermo Fisher Scientific, Waltham, MA, USA) performed with QuantiStudio3 (Thermo Fisher) using the standard fast protocol of 95° C. for 20 s followed by 40 cycles of 95° C. for 1 s and 60° C. for 20 s. Denaturation protocol consisted of 95° C. for 1 s, 60° C. for 20 s and a final dissociation step of 95° C. Relative gene expression was calculated using the method described previously 9. CsGAPDH was used as an endogenous control.

TEM analysis. Small sections of the leaf and stem samples were collected under a stereomicroscope (Swift Table Stereo Zoom Microscope, Carlsbad, CA, USA). The leaf samples were transferred to 3% glutaraldehyde overnight at 4° C. for fixation. Then, the samples were postfixed in 2% osmium tetroxide prepared in 3% glutaraldehyde for 4 h at room temperature in a fume hood. The samples were dehydrated by sequential treatment with 10%, 20%, 30%, 40%, 50%, 60%, 70%, 80%, 90% and 100% (thrice) acetone for 10 min each. The leaf samples were incubated sequentially in 50%, 75% and 100% (twice) Spurr's low-viscosity epoxy resin prepared in acetone for 8 h each. One-micrometer sections were cut with glass knives using an ultramicrotome followed by staining with methylene blue/azure A for 30 sec and basic fuchsin (0.1 g in 10 ml of 50% ethanol) for 30 sec. The sections were observed under a Leitz Laborlux S compound microscope (Leica Microsystems, Wetzlar, Germany) for the right spot with a vascular system. The same blocks were trimmed with a surgical blade and then sectioned to 0.1 m using a diamond knife under an ultramicrotome. The thin sections were collected on 200-mesh copper grids. The samples were stained with 2% aqueous uranyl acetate for 5 min, washed in water, and again stained with lead citrate followed by water wash. The micrographs were prepared and analyzed using a Morgagni 268 (FEI Company, Hillsboro, OR, USA) transmission electron microscope equipped with an AMT digital camera (Advanced Microscopy Techniques Corp., Danvers, MA, USA).

Trypan-blue staining. Trypan-blue staining was conducted as described by Fernindez-Bautista et al.¹⁰

Monitoring ROS formation and localization in phloem tissues by use of the fluorescent probe 2′,7′-dichlorodihydrofluorescein diacetate (H2DCFDA) and confocal laser microscopy. Four CLas-infected branches collected from HLB-positive C. sinensis ‘Valencia’ trees and four CLas-free branches collected from healthy C. sinensis ‘Valencia’ trees were collected and placed in glass test tube with 30 mL water containing 10 μM 2′,7′-dichlorodihydrofluorescein diacetate (H2DCFDA). Four leaves/branch at the top were kept to facilitate transpiration. Glass tubes were wrapped with aluminum foil and kept in room temperature for 24 hours. Bark was peeled from the stem section that was submerged in water and placed on slide with inner side upwards. HLB-positive and healthy branches were also incubated in water without H2DCFDA as controls. 2′,7′-dichlorofluorescein (DCF) fluorescence was visualized by confocal laser scanning microscopy (CLSM) (Leica TCS-SP5, Mannheim, Germany) with excitation/emission at 495 nm/525 nm.

Trunk injection of HLB-positive 5-year-old C. sinensis trees. Trunk injection was conducted as described elsewhere¹⁴. For each tree, approximately 0.4 g streptomycin sulfate (laboratory grade; Thermo Fisher Scientific) at 5 g/L was injected into the trunk. The amount of streptomycin injected was calculated to reach the concentration needed to kill CLas in planta based on the canopy volume¹¹.

Exudates of phloem enriched bark tissues for H₂O₂ and ion leakage assays. Exudates of phloem enriched bark tissues were extracted from stems following the procedure described elsewhere¹². Stems were collected from small branches of spring spouts with mildly symptomatic leaves.

Protoplast. Protoplast cells of C. sinensis ‘Hamlin’ were prepared as described by¹³. Embryogenic calli were subcultured on solid MT (Murashige and Tucker) media (Phytotech) every 2 weeks. From the maintained calli suspension, cells were prepared and maintained in DOG liquid media as described elsewhere¹⁴. The final isolated protoplast cells were suspended in W5 solution (154 mM NaCl, 125 mM CaCl₂), 5 mM KCl, 2 mM MES at pH 5.7) at 1×107 cells/ml for different treatments.

Treatment of protoplast cells with H₂O₂, antioxidants, and Gibberellic acid (GA) For H₂O₂ treatment, H₂O₂ was freshly prepared as 1 M stock solution with sterile double stilled water. For protoplast treatments with different concentrations of H₂O₂, H₂O₂ was further prepared as 100× stock with protoplast buffer (W5 solution). Protoplast cells were then treated with different concentrations of H₂O₂ for 24 hours. For each treatment, at least three biological replicates were conducted. After 24 h treatment, samples were stained with Fluorescein Diacetate (FDA) (Invitrogen) for viability observation. For 50 ul of sample, 2 ul of FDA was added. Immediately after staining, the samples were observed under Olympus BX53 Epi-fluorescence microscope with green channel. The ratio of green cells (living cells) to total cells was calculated as viability rate. All steps and chemical treatments were performed at room temperature.

Protoplast cells co-treated with H₂O₂, antioxidants or Gibberellic acid (Alfa Aesar) were conducted as described above. Uric acid (Thermo Fisher Scientific) was dissolved in protoplast buffer (W5 solution). Rutin hydrate (Sigma-Aldrich) was dissolved in DMSO as stock, GA was dissolved in sterile double stilled water as stock.

Treatment of Citrus Suspension Culture Cells with H₂O₂

Sweet orang Hamlin suspension culture seven days after subculture was used. Five ml of suspension culture cells were aliquoted into a 50-ml Falcon tube. Freshly prepared H₂O₂ was added into each tube at a concentration of 0 (water control), 0.6 mM, 1.5 mM, 1.8 mM, or 3.6 mM. The tubes were incubated at room temperature with gentle shaking (100 rpm). Twenty-our hours after treatment, 50 ul of cells were pipetted into a 1.5-ml tube from each treatment. Each sample was stained with 2 ul of fluorescein diacetate (stain only living cells, green color) and 2 ul of propidium iodide (stain only dead cells, orange to red color). One minute after staining, the stained samples were observed under a fluorescent microscope with green and red al channels.

Foliar spray with antioxidants and GA. Five-year-old Valencia sweet orange trees with similar symptoms were used for foliar spray treatments. All trees in the grove were HLB-positive. The experiment was a completely randomized design with 5 treatments. Each treatment consisted of four trees. The treatments were applied by foliar spray with 2.5 mL/L of Induce non-ionic surfactant (Helena Ag, Collier, TN, USA). One liter of solution per plant were applied at approximately 400 kPa using a handheld pump sprayer to apply on the whole tree. This pressure resulted a fine mist and was sufficient to produce runoff from the leaves to ensure complete coverage. Individual treatments were applied to the various trees as follows: uric acid (1.8 mM), rutin (0.6 mM), GA (5 mg/L), and GA (25 mg/L). Water was used as the negative control. Foliar spray was conducted in the evening to facilitate absorption. The chemicals GA, and uric acid were purchased from Fisher Scientific. Rutin was purchased from Sigma-Aldrich (St. Louis, MO, USA).

GA treatment via foliar spray. GA foliar spay was conducted in the first week of November, 2020. For the GA application, 20 ounces of Pro Gibb LV (Valent U.S.A. LLC, Walnut Creek, CA, USA) was mixed with water in a 100 Gal tank. 64 ounces of WIDESPREAD MAX (A.I. organosilicone) was included as the surfactant for leaf spray with airblast. Applications were conducted during night. One block of Valencia sweet orange on rootstock 942 was treated with GA, whereas the nearby Valencia/942 block was not treated with GA as a negative control. In addition, one block of Vernia sweet orange on X639 rootstock was treated with GA with one nearby Vernia/X639 block as a negative control. All blocks are 10 acres or more with approximately 140 trees/acre.

RNA-seq analyses of GA treatment on citrus protoplast cells in the presence of H₂O₂ Protoplast cells were prepared as described above and suspended in W5 solution (154 mM 683 NaCl, 125 mM CaCl₂), 5 mM KCl, 2 mM MES at pH 5.7) at 1×107 cells/ml. All steps were performed at room temperature. The following two treatments were conducted: 1) Protoplast+H₂O₂ (1.8 μmol/mL)+Gibberellin (5 mg/L), and 2) Protoplast+H₂O₂(1.8 mol/mL). Protoplast cells were maintained at room temperature without shaking. RNA was collected at 6 h after treatment. Four biological replicates were included for each treatment.

Total RNA was extracted using the RNeasy plant kit (Qiagen, Valencia, CA), followed by treatment with RQ1 RNase-Free DNase (Promega, Madison, WI). RNA concentration and quality were measured by a Nanodrop One Microvolume UV-Vis Spectrophotometer (Thermo Fisher Scientific, Waltham, MA). Samples meeting the following requirements (Concentration ≥20 ng/L, OD260/280 >2.0) were sent to Novogene (Novogene, Davis CA) for cDNA libraries construction and RNA-seq analyses. Libraries were constructed with the NEBNext Ultra II RNA 694 Library Prep Kit for Illumina (Illumina, San Diego, CA). Samples were sequenced to generate 150 bp paired-end reads using the Illumina NovaSeq 6000 platform (Illumina). Raw RNA-seq data were filtered by removing low-quality reads and adapters, and then aligned to the Citrus sinensis reference genome 77 using HISAT2 version 2.2.1 78 and SAMtools version 1.279 each gene was quantified using HTSeq-count version 0.11.280. Different expressed genes (DEGs) analysis was performed using DESeq2 packages version 1.30.1 in R version 4.0.581. Genes were considered significantly expressed with adjusted p-value <0.05 (FDR method). The heatmap plots of expression profiling of DEGs were drawn using the pheatmap package version 1.0.12 in R program version 4.0.582.

### NADPH Oxidase Inhibitor Diphenyleneiodonium (DPI) Treatment on ROS Levels in CLas-Positive Stems

HLB positive branches from the summer flush of Valencia sweet orange in the field were collected and then soaked in DPI solution (25 μM) or water (control). After 48 h treatment, phloem-enriched bark tissues were collected for H₂O₂ concentration measurement. Experiments were repeated two times and representative result is shown.

Evaluation of citrus tree growth and HLB symptoms in response to GA treatment. Tree growth was evaluated by estimating trunk diameter, tree height (TH), and canopy volume (CV) on both GA treated and untreated control trees. For each treatment group, a total of 10 trees (n=10) were randomly selected for the evaluation. A digital caliper (Fowler, Newton, MA) was used to take two measurements of trunk diameter (north-south and east-west orientation) at −20 cm above the ground. A tape measure was used to measure the TH above the ground from the soil surface to the apical point of the plant. CV was estimated by taking the average of two independent measurements of the diameter of the canopy at different directions (north-south and east-west). The CV was estimated using the equation: V=(⅔)×p×h×(d/2)2, where h is the TH and d is the average diameter of the tree canopy¹⁵. All statistical analyses were performed using SAS V9.4 (SAS Institute Inc., Cary, NC). The data were first tested for normality and homogeneity of variance using the Shapiro-Wilk's test and Levene's test, respectively. A Student's two-tailed t test was performed to explore differences between GA treated and untreated control trees in growth performance traits.

HLB disease incidence in different treatments was evaluated by randomly checking 200 trees/treatment. Ratio of symptomatic leaves vs total leaves in different treatments were investigated by evaluating 3 groups of branches/treatment with each group containing 16 branches that were selected randomly from 8 trees (2 branches/tree).

Data analyses of RNA-seq data. To generate the comprehensive expression pattern of sweet orange in response to CLas infection, 15 microarray and 9 RNA-seq data sets were collected from NCBI SRA and GEO databases (Table 2). The differentially expressed genes (DEGs) were determined using Limma¹⁶ and DESeq2¹⁷ packages in R for microarray and RNA-seq data, respectively (adjusted p value <0.05 and |log 2 fold change|>1). Gene ontology (GO) term enrichment of DEGs was conducted using agriGO v2.0: a GO analysis toolkit for the agricultural community¹⁸ using the singular enrichment analysis tool. The heatmap plots were drawn using the gplots package in R program¹⁹.

Data availability. The raw RNA-seq reads were deposited in the NCBI Bioproject databased under the accession number PRJNA780217.

### Example 1 CLas does not Contain Pathogenicity Factors that Directly Cause HLB Symptoms

A comprehensive analysis of CLas proteins was conducted and no homologs with known pathogenicity factors that are directly responsible for causing disease symptoms were identified. Ca. Liberibacter spp. do not contain the type II, III, and IV secretion systems that secrete such pathogenicity factors. To test whether CLas contains pathogenicity factors responsible for causing HLB symptoms, predicated virulence factors including serralysin and hemolysin (substrates of type I secretion system) and proteins containing Sec secretion signals (Table 1)⁷ were overexpressed in Arabidopsis thaliana, Nicotiana tabacum or Citrus paradisi. These virulence factors refer to genes that contribute to bacterial growth in plants but are not directly responsible for disease symptoms, and genes that contribute to virulence in non-plant hosts, such as seralysin. None of the overexpressed CLas proteins caused HLB-like symptoms, consistent with the bioinformatic analyses that CLas does not contain pathogenicity factors that directly cause HLB symptoms. Intriguingly, multiple characterized proteins of CLas, such as SDE1, SDE15, and SahA, suppress plant immune response⁸⁻¹⁰, suggesting that CLas triggers immune response, which, it is hypothesized, is responsible for causing the devastating damages of the HLB disease, mimicking the immune-mediated diseases of human.

### Example 2. CLas Infection Triggers Immune Response and Cell Death in the Phloem Tissues

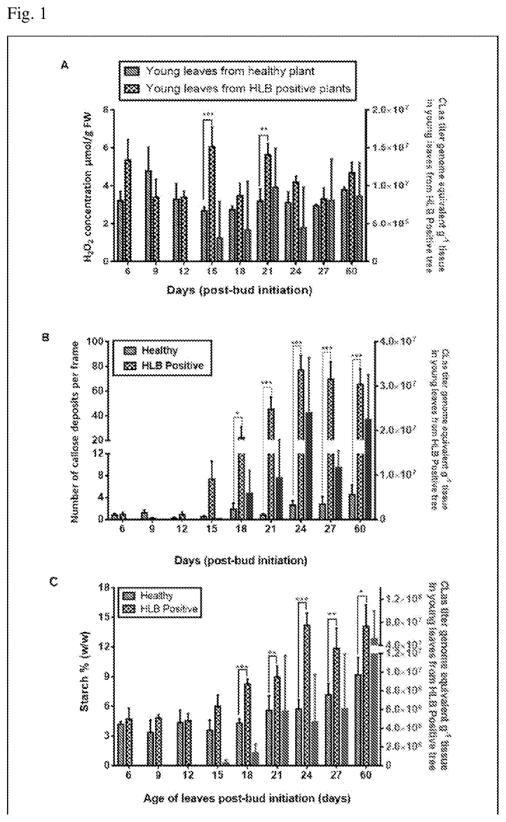

Next, whether and how CLas triggers immune response and cell death was tested. Newly emerged citrus flush from HLB positive citrus trees is free of CLas for a short period of time. HLB positive and healthy two-year-old C. sinensis ‘Valencia’ trees were trimmed in a greenhouse to trigger young flush and conducted dynamic analyses of the relationship between CLas infection, immune response, cell death, and HLB symptom development. CLas was detected in young leaves of HLB positive trees at approximately 15 days post bud initiation based on quantitative PCR (qPCR) (FIG. 1). The H₂O₂ (an indicator of reactive oxygen species (ROS)) content in CLas positive flush was significantly higher than that of healthy plants at 15- and 21-day post bud initiation (FIG. 1A). Significantly more callose deposition, an indicator of immune response¹¹, was observed in CLas positive flush than that of the healthy plants starting at 18 days post bud initiation and thereafter (FIG. 1B). On the other hand, significantly more starch accumulation was observed in CLas positive samples than in healthy samples starting at 18 days post bud initiation and afterwards (FIG. 1C). Symptoms began to appear at approximately 40 days post bud initiation. It appears that CLas infection triggered plant immune response, such as ROS (e.g., H₂O₂) production and callose deposition, followed by symptom development.

To have a better understanding of the nature of the immune response induced by CLas, temporal expression analyses of immune marker genes (PR1, PR2, PR3, and PR5) was conducted in young leaves at 15-, 18-, 21-, 24-, 27-, and 60-days post-bud initiation for the CLas infected and healthy C. sinensis plants. PR genes were consistently induced by CLas despite some fluctuations between 15- and 60-day-post-bud initiation (FIG. 7).

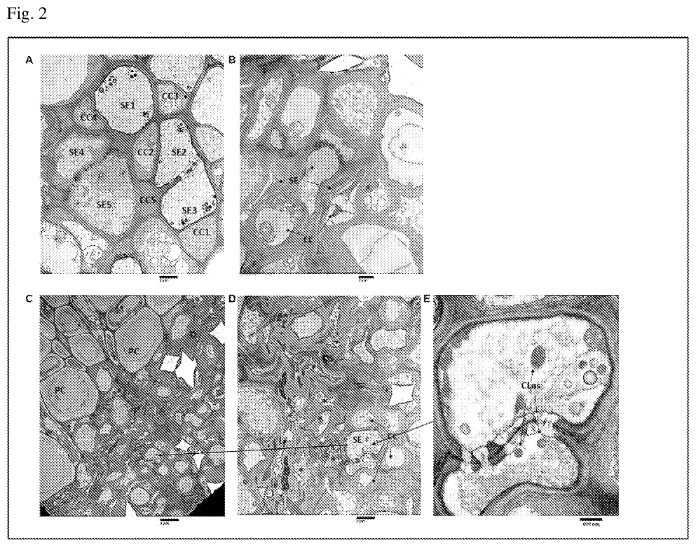

Cell death of sieve element and companion cells was observed via transmission electron microscopy (TEM) analysis of asymptomatic young leaves of HLB positive C. sinensis ‘Valencia’ trees (FIG. 2A-D), indicating cell death of phloem tissues occurs prior to the appearance of HLB symptoms. CLas was observed in phloem tissues with intact sieve element and companion cells, but not in sieve element cells undergoing the cell death process (FIG. 2C-E). Particularly, some sieve element and companion cells were observed undergoing cell death while others remained intact in the same field (FIG. 2D), explaining the reduced function of the phloem tissues, rather than a complete loss of function. This accords with the observation that CLas moves primarily vertically, but not laterally during infection¹². TEM observation of CLas infected leaves confirmed that cell death was limited to sieve element and companion cells, but not occurring in surrounding parenchyma cells (FIG. 2C).

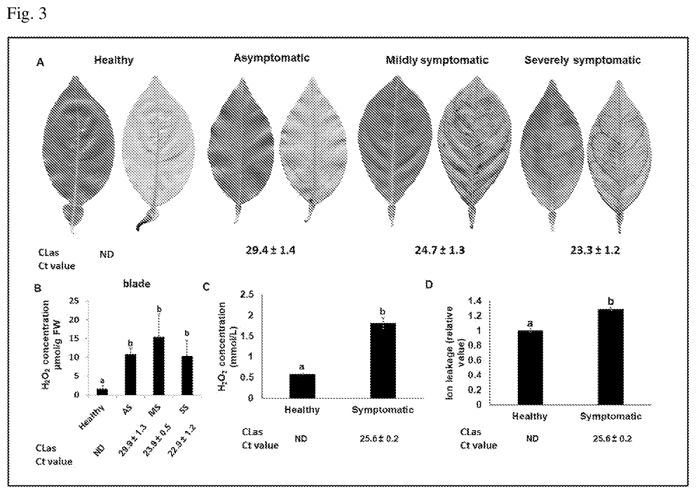

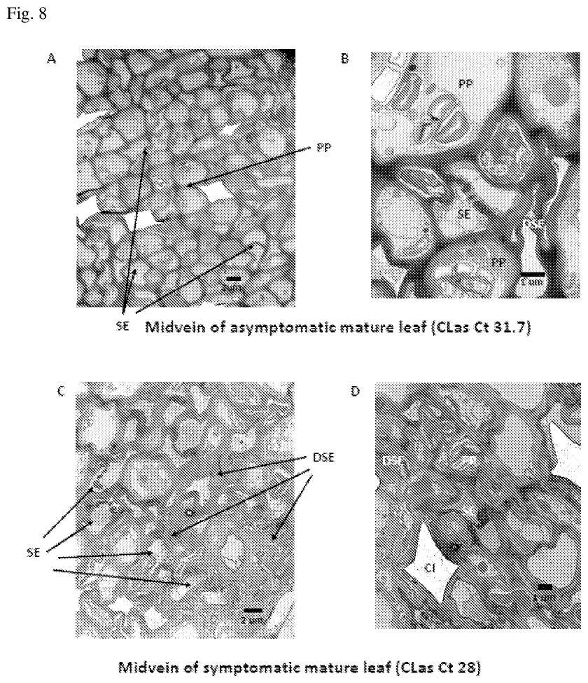

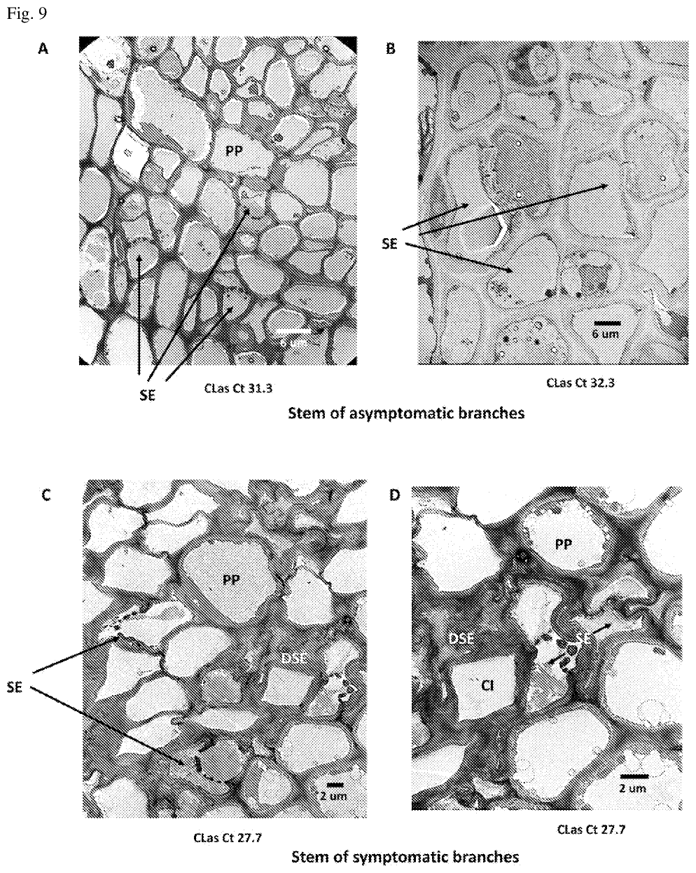

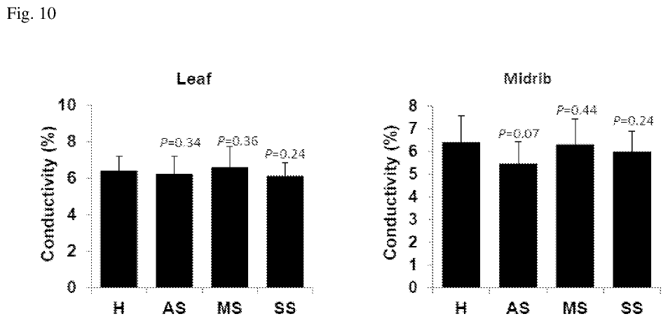

The cell death in C. sinensis mature leaves was confirmed showing different symptoms based on trypan blue staining. No cell death was observed in healthy leaves collected from CLas-free plants. Cell death was observed in CLas positive asymptomatic leaves, and leaves with mild or severe HLB symptoms and correlated positively with symptom development (FIG. 3A). In addition, cell death was observed along the vascular tissues, congruent with the TEM observation of the death of sieve element and companion cells in the midribs of CLas infected mature leaves and CLas positive stem tissues (FIGS. 8 and 9). More cell death was observed with increasing CLas titers, suggesting CLas infection is responsible for the cell death of the phloem tissues (FIGS. 2 and 3). TEM observation demonstrated that cell death of companion and sieve element cells occurred concurrently without obvious difference in timing, in agreement with their function together as a unit¹³. Significantly higher H₂O₂ concentrations was observed in CLas infected mature leaves than CLas free leaves (FIG. 3B). In addition, significantly higher H₂O₂ concentrations were detected in the exudates of phloem enriched tissues of CLas positive stems than that of stems of CLas-free trees (FIG. 3C).

Cell death is usually accompanied by ion leakage. Surprisingly, no difference was observed in ion leakage between leaf blades or midribs of healthy, asymptomatic, mildly symptomatic, and severely symptomatic leaves (FIG. 10), contrary to the TEM and trypan blue staining data (FIGS. 2 and 3A, and FIG. 8). However, the ion leakage values of the exudates extracted from phloem enriched bark tissues of CLas infected samples were significantly higher than that of healthy samples (FIG. 3D), consistent with that cell death happens in the phloem tissues, but not in surrounding parenchyma cells (FIG. 2C). The negative ion leakage data related to leaf blades and midribs of CLas infected samples (FIG. 10) probably result from the mask effect of parenchyma cells because companion and sieve element cells make up only approximately 1% of the total cell population in plants¹⁴.

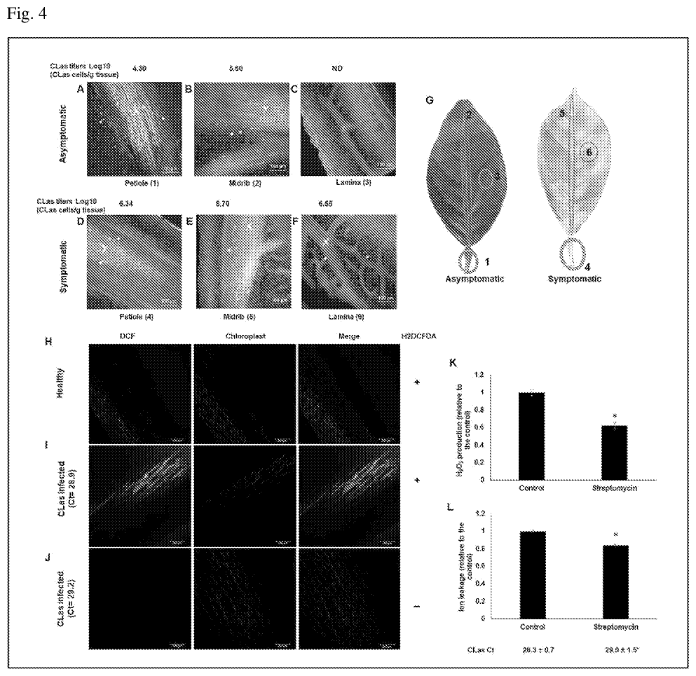

Next, callose deposition was used as an indicator to investigate the localization of the immune response in citrus leaves in response to CLas infection. For this test, callose deposition was investigated in different sections of asymptomatic and symptomatic leaves of HLB-positive C. sinensis trees (FIG. 4A-G). The callose deposition in the petiole, midrib, and lamina of asymptomatic leaves was significantly lower than that in their counterparts of symptomatic leaves (FIG. 4, A-G). No callose deposition was observed in the CLas-free lamina of asymptomatic leaves (FIG. 4C). Collectedly, the correlation between callose deposition and CLas titers suggests that CLas is responsible for inducing callose deposition (FIG. 4A-F) as observed in the past 15. Callose deposition was observed only in the phloem tissues as observed previously¹⁶, but not in mesophyll cells. In contrast, for pathogens infecting the apoplast, such as Xanthomonas, callose is deposited between the plasma membrane and the cell wall at the site of pathogen attack^{17, 18}. Hence, CLas induces systemic immune response in the phloem tissues following systemic CLas infection.

To further verify that CLas induces immune response in the phloem tissues, ROS formation and localization was monitored in phloem-enriched bark tissues using the fluorescent probe 2′,7′-dichlorodihydrofluorescein diacetate (H2DCFDA) and confocal laser microscopy. H2DCFDA is a commonly used cell-permeable probe for measuring cellular H₂O₂¹⁹. Significantly higher H₂O₂ was detected in the phloem-enriched bark tissues of CLas infected citrus plants than that of CLas-free plants (FIG. 4H-J).

Next, the causal relationship between CLas infection and ROS induction and cell death in phloem tissues was further established. For this assay, CLas-positive 5-year-old C. sinensis trees were treated with streptomycin to kill CLas via trunk injection²⁰. At 7 days post treatment, streptomycin significantly reduced CLas titers, H₂O₂ content and ion leakage in the phloem tissues (FIG. 4K-L). Collectively, the causative relationship has been established between CLas infection and ROS induction and cell death in the phloem tissues.

### Example 3. HLB Caused Cell Death is Instigated by ROS

It is known that cell death can be initiated by ROS²¹. At high concentrations, ROS triggers necrotic cell death, but induces programmed cell death below the ROS threshold²² Next, H₂O₂ contents were analyzed in C. sinensis leaves triggered by CLas infection. The H₂O₂ concentration induced by CLas infection in young leaves was approximately 6 μmol g⁻¹ FW, but reached 10-15 μmol g⁻¹ FW in mature leaves (FIG. 3B). Moreover, this method probably underestimated the H₂O₂ concentration in the phloem tissues because it could not differentiate phloem cells, where H₂O₂ concentrates (FIG. 4H-J), from parenchyma cells. Intriguingly, Xanthomonas citri subsp. citri, another bacterial pathogen of citrus, infection of kumquat (Citrus japonica, syn: Fortunella crassifolia) triggers H₂O₂ production at 2 days after inoculation which peaks (9.86 μmol g⁻¹ FW) at 8 days after inoculation, eventually leading to cell death^{23, 24}. X, citri subsp. citri induced cell death is a slow process and happens at approximately 6-8 days after inoculation²⁴. Hence, it was conjectured that ROS induced by CLas reaches the threshold necessary to trigger the death of companion and sieve element cells in mature leaves, but possibly not in the early stage of infection of young leaves before CLas titers reach a certain threshold. The ROS production triggered by CLas is distinct from that triggered by incompatible pathogens, which is typified by a biphasic oxidative burst²⁵. Instead, the ROS production triggered by CLas is chronic, and was observed in young leaves during early infection stages as well as in CLas infected mature leaves consistently, probably triggered by CLas colonizing and multiplying in the previously unoccupied phloem tissues.

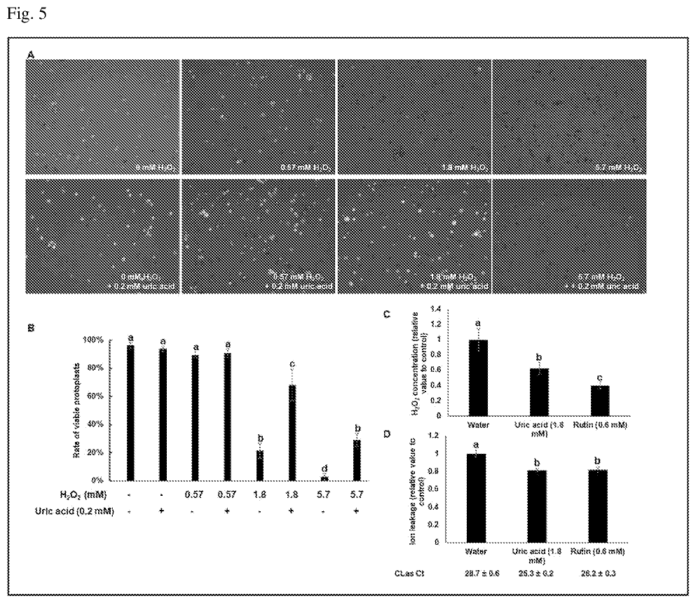

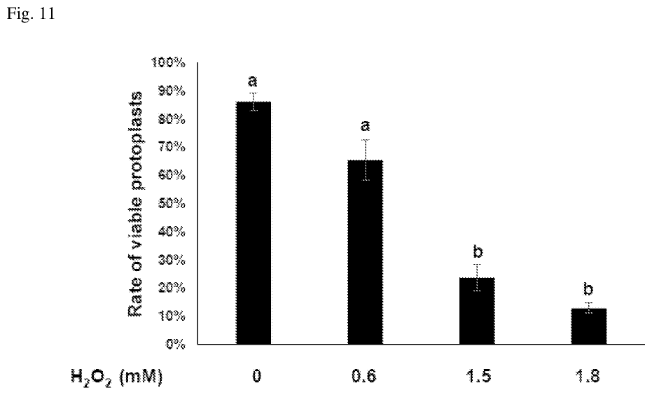

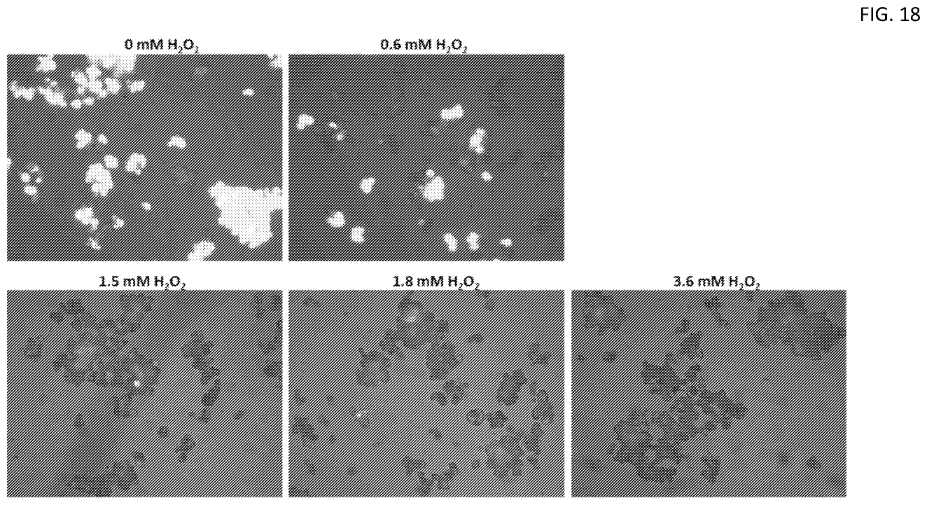

Furthermore, the H₂O₂ concentrations in the exudates extracted from the phloem enriched bark tissues from symptomatic (1.80±0.13 mmol/L) CLas positive branches were significantly higher than that (0.59±0.01 mmol/L) of healthy trees (FIG. 3C). H₂O₂ induces necrosis of immortalized rat embryo fibroblasts at a concentration of 700 μmol/L 26. H₂O₂ at concentrations of 1.8 mmol/L but not 0.6 mmol/L or lower induced cell death of C. sinensis protoplast cells (FIGS. 5A and B; FIG. 11). Similar results were observed for suspension culture cells (FIG. 18) Addition of uric acid (0.2 mM), a ROS scavenger, reduced both H₂O₂ concentration (FIG. 5C) and cell death (FIGS. 5, A and B), indicating H₂O₂ induced by CLas alone can cause cell death of phloem tissues. It is important to note that growth of plants (e.g., Arabidopsis) is inhibited by 1 mM H₂O₂²⁷, partly explaining the growth stunting phenotype of CLas infected young trees.

In addition to H₂O₂, ROS induced by pathogens include hydroxyl radicals, superoxide anions, and singlet oxygen. To further corroborate that HLB caused cell death is instigated by ROS, weekly foliar spray of HLB positive C. sinensis ‘Valencia’ trees was conducted with antioxidants uric acid (1.8 mM) and rutin (0.6 mM). Six weeks later, analysis of the exudates extracted from the phloem-enriched bark tissues demonstrated that both uric acid and rutin treatments reduced both ROS production, as indicated by H₂O₂ concentration (FIG. 5C), and cell death (FIG. 5D). Taken together, CLas infection of citrus phloem tissues induces ROS production, which subsequently causes cell death of phloem tissues.

### Example 4. CLas Infection Significantly Affects Pathways Related to Oxidative Stress and Immune Responses

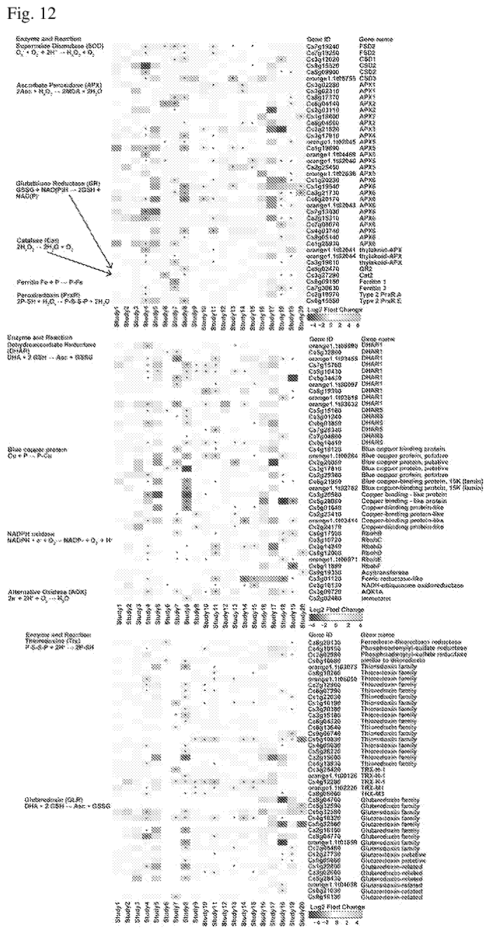

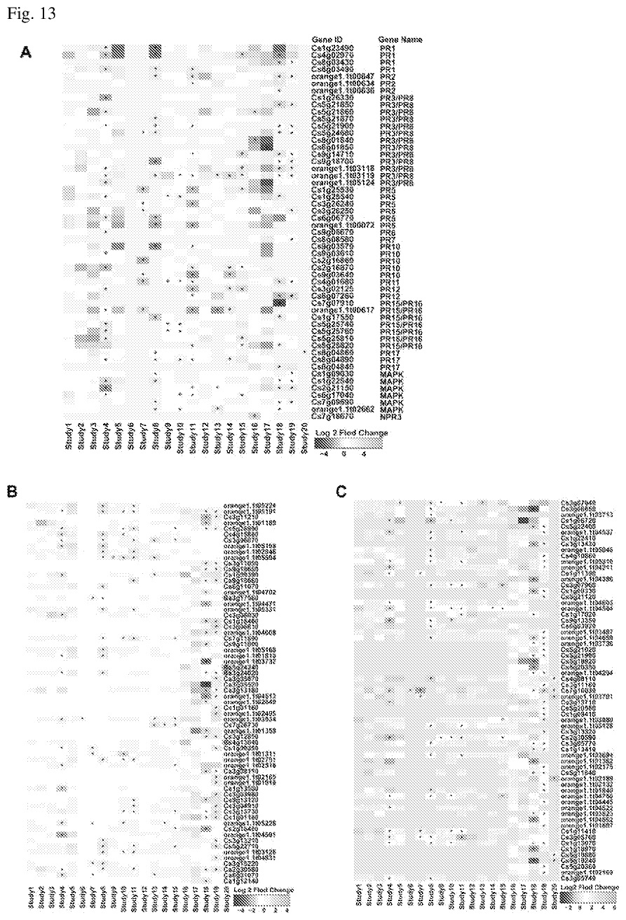

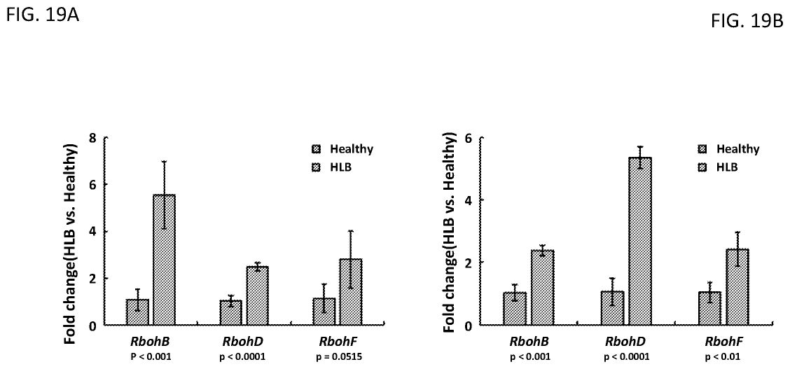

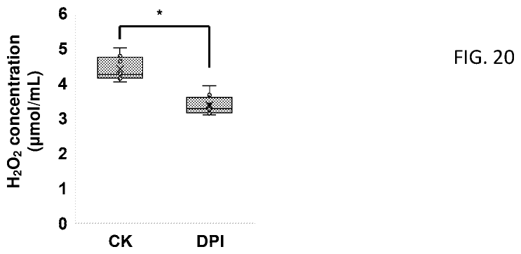

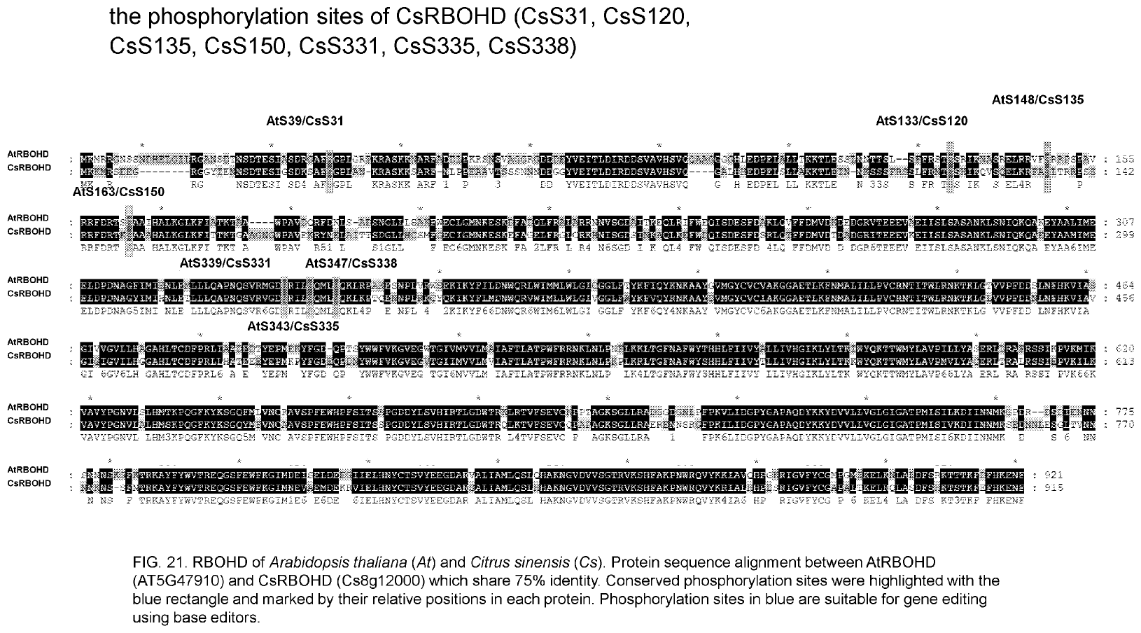

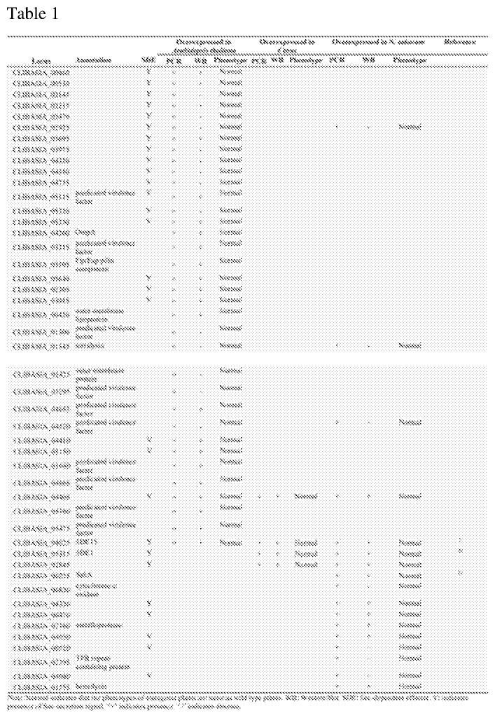

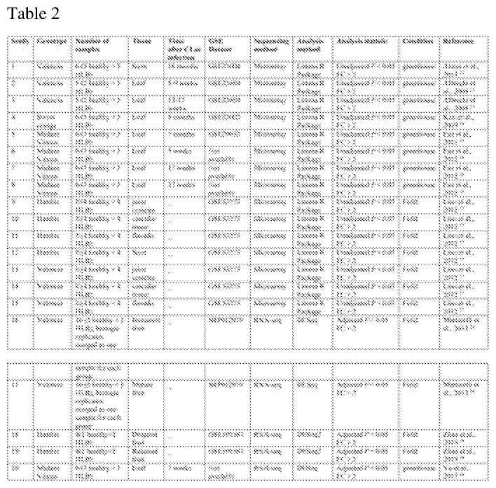

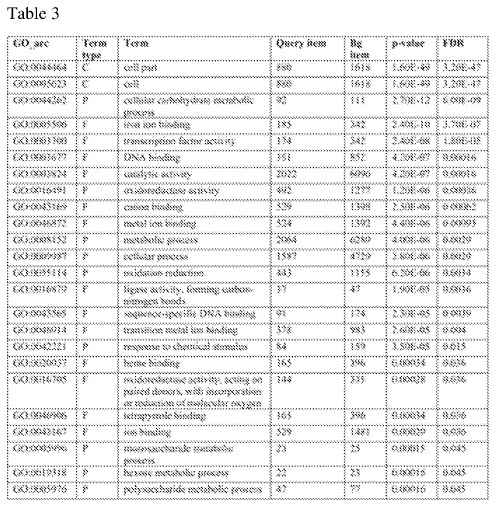

Next, the gene expression profiles of C. sinensis was investigated in response to CLas infection that were conducted previously, comprising 9 studies including different tissues (leaf, stem, and fruit), different environments (greenhouse or groves), and different infection stages (Table 2). Enrichment analyses of differentially expressed genes (DEGs) clearly demonstrated that the expression of genes related to ROS and immune response are significantly affected by CLas infection (Table 3). The combined analysis showed an overall downregulation of antioxidant enzymes and upregulation of transmembrane localized NADPH oxidases, known as RBOHs, explaining the oxidative stress response in response to CLas infection (FIG. 12). Critically, expression of respiratory burst oxidative homolog D (RbohD) genes, which encode an enzyme implicated in the generation of ROS during the defense response, was induced by CLas infection in most occasions. RBOHD is a main producer of ROS upon PAMP recognition and required for cell death initiated after pathogen detection²⁸. In addition, ROBHB and RBOHF have also been reported to be involved in ROS production in response to pathogen infection xx. qRT-PCR assays revealed that both RBOHB and RBOHD were induced by CLas infection of leaf samples collected in both field and greenhouse conditions, whereas RBOHF was induced only under field conditions (FIG. 19). Intriguingly, ROS levels in CLas-positive stems were reduced by NADPH oxidase inhibitor diphenyleneiodonium (DPI) (FIG. 20), supporting the notion that CLas triggers ROS production via RBOH genes. The combined analysis implies that CLas infection causes oxidative stress to citrus in most conditions, consistent with H₂O₂ production triggered by CLas (FIGS. 1A, 3B-C, 4H-K, and 5C). The combined analysis also revealed complex expression changes associated with the immune response pathways in response to CLas infection (FIG. 13). Intriguingly, approximately 66 NLR genes showed overall induction by CLas in multiple studies (FIG. 13B). In sum, transcriptome analyses of sweet orange in response to CLas infection support our hypothesis that HLB is an immune-mediated disease that results from ROS induced cell death of phloem tissues triggered by CLas.

### Example 5. Suppressing ROS Mediated Cell Death Mitigates HLB Symptoms

Antioxidants, and immunoregulators are commonly used to treat human immune-mediated diseases by halting or reducing ROS mediated cell death²⁹⁻³¹. Correspondingly, it was tested whether growth hormones gibberellin (GA), and antioxidants (uric acid and rutin) mitigate ROS mediated cell death triggered by CLas infection, thus blocking or reducing HLB symptoms. GA is selected because it is a known plant growth hormone and modulates PAMP-triggered immunity and PAMP-induced plant growth inhibition³². Both uric acid and rutin are well-known ROS scavengers^{33, 34}.

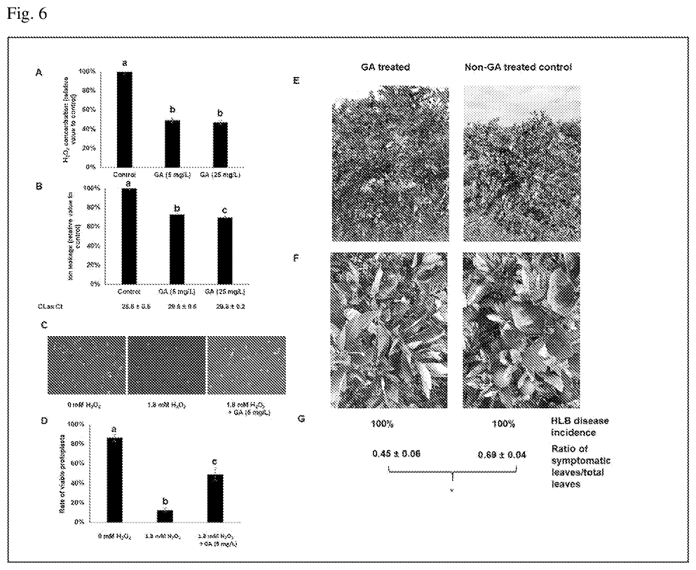

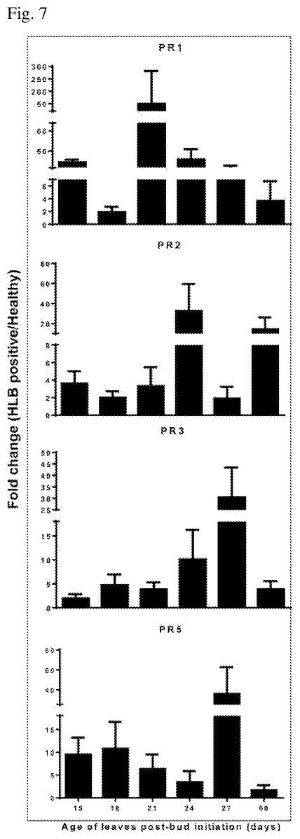

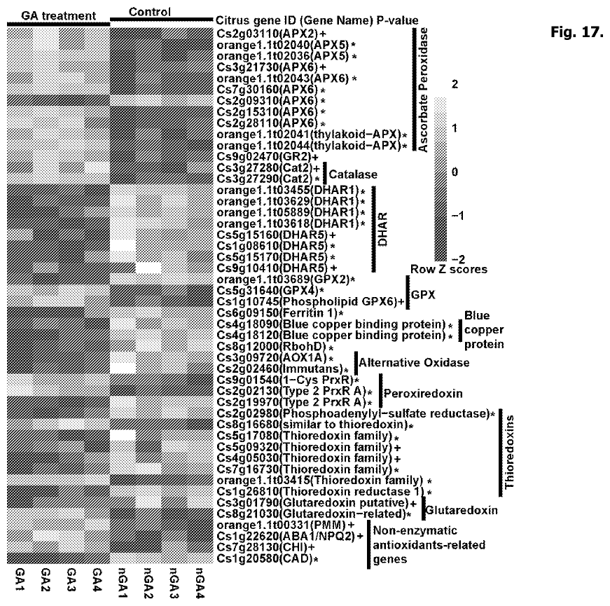

Foliar sprays of HLB positive C. sinensis trees with GA at both 5 mg/L and 25 mg/L reduced H₂O₂ and ion leakage caused by CLas (FIGS. 6, A and B). Consistent with the foliar spray results, GA (5 mg/L) also suppressed death of C. sinensis protoplast cells caused by 1.8 mM H₂O₂(FIGS. 6, C and D). RNA-seq analyses of GA treated vs. non-GA treated C. sinensis protoplast cells in the presence of 1.8 mM H₂O₂ demonstrated that GA induced the expression of genes encoding H₂O₂ scavenging enzymes catalases, ascorbate peroxidases and glutathione peroxidases. GA also inhibited the expression of RbohD (FIG. 17). GA treatment therefore clearly alleviates the oxidative stress caused by H₂O₂ in citrus.

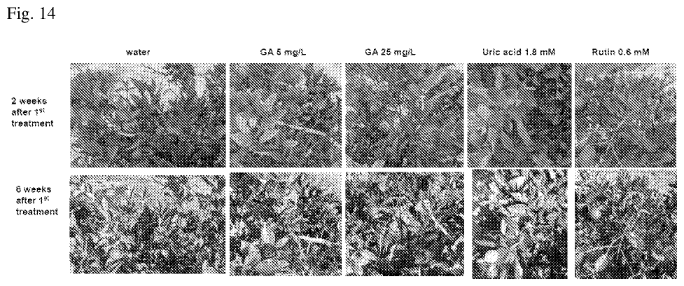

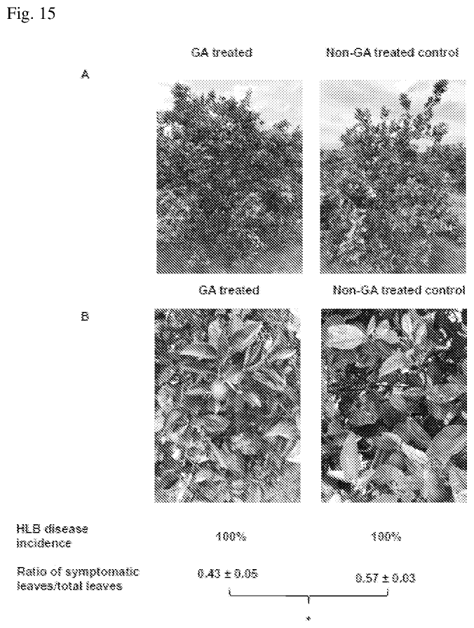

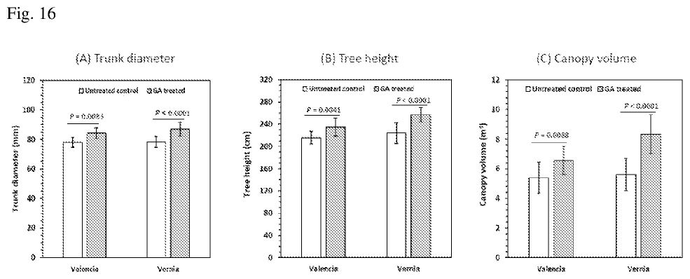

Six weeks after foliar spray, the treated plants showed reduced HLB symptoms (i.e. less blotchy mottle) compared with that before treatment, whereas plants with water treatment developed more severe HLB symptoms in the same period (FIG. 14). In addition, it was obvious that GA promoted growth as indicated by new flushes, which were not observed for the water control in the same duration (FIG. 14). GA is registered to be used on citrus in the US, which enables large scale field trials to test its effect against HLB. Foliar spray of C. sinensis with GA significantly reduced HLB disease symptoms 8 months after application (FIGS. 6, E and G, and FIG. 15). The treated trees looked much healthier than the non-treated control despite both were 100% infected (FIG. 6G). Because symptomatic leaves demonstrated significantly more cell death than asymptomatic leaves based on trypan blue staining (FIG. 3a) and TEM observation (FIG. 8), the ratio of symptomatic leaves vs total leaves was used as an indicator of cell death caused by HLB. GA treatment significantly reduced the percentage of symptomatic leaves (FIGS. 6, E and G, and FIG. 15), indicating reduced cell death of sieve element and companion cells in treated leaves. In addition, foliar spray of GA on HLB positive 6-year-old C. sinensis var. ‘Valencia’ and var. ‘Vernia’ significantly promoted plant growth including tree height, trunk diameter and canopy volume at eight months after application (FIG. 16). It is probable that GA reduces HLB symptoms via its direct effect on both mitigating ROS (FIG. 6A) and promoting plant growth (FIG. 16).

To determine whether mitigating ROS can directly halt or reduce HLB symptoms, weekly foliar spray of antioxidants uric acid (1.8 mM) and rutin (0.6 mM) was conducted on HLB positive C. sinensis. Remarkably, 6 weeks after the first treatment, both antioxidants significantly reduced HLB symptoms compared to that before treatment, whereas the plants treated with water became more symptomatic in the same duration (FIG. 14). Taken together, the data suggest that suppression of ROS mediated cell death mitigates HLB symptoms. Consequently, the causal relationship that CLas triggers ROS production was established in the phloem tissues, which subsequently causes cell death of phloem tissues, leading to HLB symptoms.

### Example 6: Editing of Plant Antioxidant Enzymes

Genetic improvements that enhance plant tolerance of oxidative stress is achieved by companion cell- or phloem-specific overexpression of antioxidant enzymes (such as superoxide dismutase, catalases, glutathione peroxidases, ascorbate peroxidase, glutathione reductase, and glutathione S-transferase using CRISPR gene editing, transgenic, or cisgenic approaches or Citrus tristeza virus vectors. In certain embodiments, the promoter regions of genes encoding antioxidant enzymes are specifically edited to activate their expression in response to CLas infection.

The following three approaches are followed to enhance tolerance to oxidative stress:

1. Gene expression of antioxidant enzyme genes can be driven by 35S promoter, or phloem specific AtSUC2 promoter or the promoter of the citrus homolog of AtSUC2 gene.

2. The expression in citrus can be individually or stacking multiple genes together. For stacking, there are many combinations. Here are some examples: one SOD gene plus one catalase gene, or one SOD gene plus one catalase gene plus one APX gene, or one catalase gene plus one APX gene.

3. Gene expression of antioxidant enzyme genes can be expressed using the CTV vector. Mutations that drive overexpression of antioxidant enzymes or promote induction as a result of CLas infection may be tested by inoculating the edited plants with CLas through grafting and psyllid feeding. The effects of the editing of the promoter or coding region of antioxidant enzymes on CLas titers, ROS production, cell death in the phloem, and HLB symptom development are determined via the techniques described in Examples 1-5 above. Effects of editing on horticultural traits and fruit yield and quality is also determined.

### Promoter Sequences for Engineering Overexpression or CLas Induction

CaMV 35S promoter
(SEQ ID NO: 1)
CCTGCAGGTCAACATGGTGGAGCACGACACACTTGTCTACTCCAAAAATATCAAAG
ATACAGTCTCAGAAGACCAAAGGGCAATTGAGACTTTTCAACAAAGGGTAATATCC
GGAAACCTCCTCGGATTCCATTGCCCAGCTATCTGTCACTTTATTGTGAAGATAGTG
GAAAAGGAAGGTGGCTCCTACAAATGCCATCATTGCGATAAAGGAAAGGCCATCGT
TGAAGATGCCTCTGCCGACAGTGGTCCCAAAGATGGACCCCCACCCACGAGGAGCA
TCGTGGAAAAAGAAGACGTTCCAACCACGTCTTCAAAGCAAGTGGATTGATGTGAT
AACATGGTGGAGCACGACACACTTGTCTACTCCAAAAATATCAAAGATACAGTCTC
AGAAGACCAAAGGGCAATTGAGACTTTTCAACAAAGGGTAATATCCGGAAACCTCC
TCGGATTCCATTGCCCAGCTATCTGTCACTTTATTGTGAAGATAGTGGAAAAGGAAG
GTGGCTCCTACAAATGCCATCATTGCGATAAAGGAAAGGCCATCGTTGAAGATGCCT
CTGCCGACAGTGGTCCCAAAGATGGACCCCCACCCACGAGGAGCATCGTGGAAAAA
GAAGACGTTCCAACCACGTCTTCAAAGCAAGTGGATTGATGTGATATCTCCACTGAC
GTAAGGGATGACGCACAATCCCACTATCCTTCGCAAGACCCTTCCTCTATATAAGGA
AGTTCATTTCATTTGGAGAGGACCTCGACCTCAACACAACATATACAAAACAAACG
AATCTCAAGCAATCAAGCATTCTACTTCTATTGCAGCAATTTAAATCATTTCTTTTAA
AGCAAAAGCAATTTTCTGAAAATTTTCACCATTTACGAACGATA
AtSUC2 promoter
>AtSUC2 promoter region
(SEQ ID NO: 2)
GCAAAATAGCACACCATTTATGTTTATATTTTCAAATTATTTATTACATTTCAATATT
TCATAAGTGTGATTTTTTTTTTTTTTGTCAATTTCATAAGTGTGATTTGTCATTTGTAT
TAAACAATTGTATCGCGCAGTACAAATAAACAGTGGGAGAGGTGAAAATGCAGTTA
TAAAACTGTCCAATAATTTACTAACACATTTAAATATCTAAAAAGAGTGTTTCAAAA
AAAATTCTTTTGAAATAAGAAAAGTGATAGATATTTTTACGCTTTCGTCTGAAAATA
AAACAATAATAGTTTATTAGAAAAATGTTATCACCGAAAATTATTCTAGTGCCACTT
GCTCGGATCGAAATTCGAAAGTTATATTCTTTCTCTTTACCTAATATAAAAATCACA
AGAAAAATCAATCCGAATATATCTATCAACATAGTATATGCCCTTACATATTGTTTC
TGACTTTTCTCTATCCGAATTTCTCGCTTCATGGTTTTTTTTTAACATATTCTCATTTA
ATTTTCATTACTATTATATAACTAAAAGATGGAAATAAAATAAAGTGTCTTTGAGAA
TCGAACGTCCATATCAGTAAGATAGTTTGTGTGAAGGTAAAATCTAAAAGATTTAA
GTTCCAAAAACAGAAAATAATATATTACGCTAGAAAAGAAGAAAATAATTAAATAC
AAAACAGAAAAAAATAATATACGACAGACACGTGTCACGAAGATACCCTACGCTAT
AGACACAGCTCTGTTTTCTCTTTTCTATGCCTCAAGGCTCTCTTAACTTCACTGTCTCC
TCTTCGGATAATCCTATCCTTCTCTTCCTATAAATACCTCTCCACTCTTCCTCTTCCTC
CACCACTACAACCACCGCAACAACCACCAAAAACCCTCTCAAAGAAATTTCTTTTTT
TTCTTACTTTCTTGGTTTGTCAAA
Coding sequences of antioxidant enzyme genes for editing to
promote over expression
superoxide dismutase (SOD) genes
>Cs3g12000
(SEQ ID NO: 3)
ATGGTGAAAGCAGTTGCAGTTCTTGGTGGAACTGAGGGTGTCAAGGGAACTGTTTCC
TTTTCCCAGGAAGGAGATGGTCCAACAACTGTATCAGGAAGCCTCTCTGGTCTCAAG
CCTGGTCCTCATGGATTCCATGTTCATGCTCTTGGAGACACAACAAATGGTTGCATG
TCTACCGGACCCCACTTTAACCCTGCTGGAAAAGAACATGGACCTCCAGAGGATGA
GAATCGTCATGCTGGTGACTTAGGAAATGTCAATGTTGGTGATGATGGTACTGCTAC
TTTTACAGTTGTTGACAATCAGATTCCTCTTTCTGGACCAAATTCCATTATTGGAAGG
GCTGTTGTAGTCCACGCAGATCCCGATGATCTTGGCAAGGGCGGTCATGAGCTGAGC
AAAACCACTGGAAATGCTGGTGGCAGAGTAGCTTGCGGCATCATTGGCCTCCAAGG
ATGA
>Cs3g12020
(SEQ ID NO: 4)
ATGGTTAGTTGGGATAAGGACAAATATTGTGGCACTATACCTCAATTGCAACATAGA
CACGCGGGGCCTTACAACAATGCTTGTCAAACACGCGTGGGAGGGAGGTTCACCGA
TCTAGAGCCTGATGGTGCTTATGCACTCGTTATTCACCAGTACGGAGACATAAGGGA
CGGTTGGAAATCAACTGGAGACCGTTTTAAACCTGCTGGAATTGCCGGTGTTATAGC
ACATTTCGTTGCTGATGATGAGGGTACGGCTCGTTTTGACCTTGGGACGAAGGCAGA
TCTTTCTGGAGAAAATTCCGTTATGAGTAGGGGTCTTGTACTCCATGTTTTTGACAGT
TCAAATCCTTGTCTGAACGTTTCCTCGGGTTCAATTCCTCGTCCCGACATAAATTGGG
ATAAGTATTTAGGTCCCGGGGTGGATTGGGCCGTCATCGGTCTCGCTCGTTGA
>Cs3g12040
(SEQ ID NO: 5)
ATGGAGAAAGCAGATTCACTAAAGTTGGTAGCCCTTTTAGCTGTTTTATTTTGTTTTG
TCAATATTGCAACCAATTCAAGACCACATAGCAAAGCAATTGCTGTTCTTGACGGCA
GTGAGGGTTTCAAGGGAACTGTCTCCTTCTACCAGGAAGATGGCCATCATACAGTCA
TAACTGTAAACCTCGCTAATCTTAAGGCCGGTTCTCATGGATTCCATGTCCACGAAT
CCGGATTCATTCGTGCTACTACAGTGGGAAACATTTTAACCCATTTAACAAATCTCA
TAGTGACATTCACGACCATGAGGAGCATCGTCATTATGGTGATTTAG
>Cs3g12050
(SEQ ID NO: 6)
ATGGAGAGAACAGCTTCAATCAAGTTGGTAGCCCTTTTAGCTGTTTTATTCTGTTTTG
TCAATGCAACCAAATCAAGAGGAGTCCCAACGGTGAAAGCAGTTGCTTATGTTATTA
GCAAATGGGGTCCCAAGGGAATTCTCCATTTTAAACAGGAAGGAGATGGCCCAACA
ACTATAAAGGGGACACTCTATTACCTTTCGCAGGGTGCTCACGGATTTCATATACAC
GTCTACGGGGACATGGCCCATTTCTGCCAGTCAACTGGCGATCACTTTAACCCATTT
CGCAAACATCATGGTGGCCCAGAGGATTGGATTCGTCATGCCGGTGATTTAGGAAAT
ATCTATGTTGGTTTTGATGGTAAAGCCAAGATCAACATTGTTGACAAGCATAAGCTG
ATTCCTCTTGTTGGGCCAAATTCCATTATCGGAAGATCTGTCGTGATACACGACAAG
CATGATGATCTTGGCAAGGTTGGAAATTACCACAGCAAAAAGAATGGAAATGCTGG
TGACAAAATAGCTTGTGGCGTCATTGGTCTCCAAGCATGA
>Cs3g12080
(SEQ ID NO: 7)
ATGGTGAATGCAACTGTAGTTCTTACTGGAGTTGTGGGTCGTGTTGGAACTATTTCCT
TTACCCAGGAAGGAAATGGTACTGCTACTTTTAAAATTTACGGGAAGGTTGCTCTTT
CTGGACCAGATTCTGTTATTGGAAGGGGTCTTGTACTCCACTCAAATCCCAGTCCAA
ATCCCGGTCCCGGAGTAGCTTGGGGTGTCATCGGTCTCGATGCATGA
>Cs3g12090
(SEQ ID NO: 8)
ATGGAGAAAGCAGCTTCACTCAAGTTAACTTTATTGGTAGCAGTTTTATTCTGTTTTG
TCAATTCAACCAAATCAACAGGAGTACCACATGGCAATAAGGTGAATGCAATTGCC
GTTATTACTGGCAGAGAGGGGGGTCCCAAGGGAAGCATCTTTTTTTATCAGGATGGA
GATCATGGTCCAACAATTTTAAATGGATACCTCCACGGCCTTCCGCCTGGTCATCAC
GGATTTCATGTTCACGCCGCCGGGGACACAAGACACGAATGCAATTCAGCTGGGAG
TCATTTTAATCCTCATAACATGTTACATGGTAGCAAAGAAGACGAGCATCGTCATGC
TGGCGATTTAGGAAATCTCATTGTTGATGCTTATGGTAATGCTTATTTGAGCCATTTT
TTAGACAATAAGATTCGTCTTACAGGGCCACATTCCATTATTGGAAGGGCTATCGTG
ATACACAAAGATCAAGATGACTTTGGCAGGGGTGGCCATAACGACAGCAAATCCAC
AGGACATGCTGGCGAAAGAATAGCTTGCGGCGTCATCGGTCTTCTTTCATATTAA
>Cs5g26750
(SEQ ID NO: 9)
ATGGAAACAGGGGCTACAAAAGCCACAGTGAAAGCTGTGGCTCTCATTTCTGGTGC
TACCAGCGTTAAAGGCTCTCTTCACTTTGTTCAAGGACCCAACGGAGTTACACATGT
GAAAGGGAAGATTACAGGACTGAAGCCAGGCCTTCATGGCTTCCATATTCACGCCCT
CGGTGACACCACTAATGGTTGCAATTCTACTGGACCTCATTTCAATCCATTGAAGAA
GGATCATGGGGCTCCCAGTGATAACGAGCGGCACGCTGGTGATTTGGGAAACATTG
TTGCAGGCCCTGATGGAGTTGCTGAGGTTTCAATTGCTGACAGGATGATTCCTCTGA
CCGGGCAACACTCAATATTGGGGAGGGCTGTTGTTGTGCATGCTGATCCTGATGATC
TTGGAAAAGTTTCAGGTACATTGGATTCTTTACAACTACTTTGA
>Cs8g15520
(SEQ ID NO: 10)
ATGCAAGCCGCAATAGCAGCCATGGCTCACGTTCTCCTCGTAGCAGCACCGCCCTCT
TCATCTCACACACTCCTTGCCACATTGCCAAATCCTAGCAGTATCACGCGCTCTTCCT
CTTCTTCTTCTTCTCATTCTCCCTCGCTTCACTCTGCATTCCACGGCGTCTCCCTCAAA
TTCCCCTCTCGTCTCAACTTATCTCTCGCCGCCGTGGCCTCCAAGAAGCCGTTGACTG
TCGTTGCTGCCGCCAAGAAAGCGGTGGCTGTTCTTAAAGGCACTTCCAATGTTGAAG
GAGTTGTTACGTTGACCCAAGAAGATGGCGGTCCTACGACTGTCAATGTTCGTGTTA
CTGGTCTTACTCCTGGGCCTCACGGGTTCCACCTACACGAATATGGTGATACGACAA
ATGGGTGTATGTCAACAGGAGCACATTTCAATCCTAATAACATGACACACGGTGCTC
CTAAAGATGAAGTGCGACACGCGGGTGACCTGGGAAACATAGTTGCTAACGCCAAT
GGTGTGGCAGAAGCAACAATTGTGGACAACCAGATATCACTGGATGGTCCAAACAC
AGTAGTTGGAAGAGCCTTTGTGGTTCATGAGCTTGAGGATGACCTTGGAAAAGGTG
GGCATGAACTTAGTCTAACCACTGGCAATGCAGGTGGAAGACTGGCATGCGCATCC
TCTATACTCCTGATTGAGTTCCAGTGGCACAAAGATTCATATGGAATAGGTTACAAA
CTGTTGCTTGAAATATGA
glutathione reductase (GR) genes
>orange1.1t02665
(SEQ ID NO: 11)
ATGGCAACTTCTGTTACAACTCCAAAGCTCACAACAACCTTCTATAAGAGACTACCA
ATTACAGTCCCACTCTCTTCTCACTTTCTTTCGCTACAAAATACTCTAACTCCATTCTC
TCACAATTGCTGCTCGCTTCTTTATTTTCAATGCCAACGCCGCCACGTTGCGGCTCGA
GCCAAGTCTGATAATGGAGCTGAGCTGCCGAGCCACTACGACTTTGACCTCTTCACT
GTTGGTGCCGGCAGCGGTGGCGTCCGCGCCTCCCGTTTTGCCGCCAATTTTGGCGCT
TCTGTGGCCATTTGTGAACTTCCATTTTCCACCATTTCTTCCGAAACCACGGGCGGCG
TTGGCGGCACGTGTGTACTTCGTGGATGTGTGCCAAAGAAGCTTTTGGTGTACGCGT
CAAAATTTTCTCATGAATTCGATGAGAGTAATGGTTTTGGATGGAAATATGGGACGG
AACCGCAGCATGATTGGAGCACTTTGATTGCTAACAAAAATGCTGAGTTGCAGCGCC
TTACCGGTATCTACAAGAACATTCTAATCAATGCTGGCATTACCTTAATTGAAGGTC
GTGGAAAGATTGTGGACCCACACACTGTTGATGTGGATGGGAAACTGTATTCAGCTA
GGCACATACTGATTTCGGTTGGGGGACGGCCCTTCATTCCTGACATTCCTGGCAGTG
AATATGCAATAGATTCTGATGCTGCCCTTGATTTGCCCTCGAAGCCTGAGAAAATTG
CAATTGTTGGTGGTGGATACATTGCCTTGGAATTTGCTGGCATCTTCAGTGGTCTAAC
AAGTGAGGTCCACGTATTTATACGGCAGAAGAAAGTTTTAAGGGGTTTCGATGAAG
ATATACGAGATTTTGTTGCTGAACGGATGTCTCTGAGAGGAATTGAGTTTCACACTG
AGGAGTCACCTCAGGCTATCCTGAAATCAACAGACGGTTCACTATCTGTGAAGACTA
ATAAAGGAACAGTTGATGGCTTCTCTCATGTAATGTTTGCAACTGGACGCAGGCCTA
ATACAAAGAACTTAGGTTTGGAGAAAGTGGGGGTCAAAATGACCAAGAATGGAGCT
ATAGAGGTTGATGAATATTCTGGTACAGCAGTTCCTTCCATTTGGGCAGTTGGAGAT
GTTACAGACAGAATAAATTTGACTCCAGTGGCTTTAATGGAGGGAGGGGCTCTAGC
AAAAACTTTGTTTCAGGCTGAGCCAACAAAACCTGATTACAGCGCTGTTCCATCTGC
AGTCTTTTCTCAGCCACCAATTGGACAAGTTGGTCTTTCTGAAGAACAGGCTATACA
AGAATATGGTGACATTGATGTCTTCACAGCAAATTTCAGACCACTAAAAGCTACTCT
CTCTGGGCTTCCAGACAGGATCTTCATGAAACTTGTATTATGTGCAAAGACGAACAA
AGTTCTGGGTTTGCACATGTGTGGAGAAGATGCACCTGAAATTGTGCAGGGATTTGC
TGTTGCTGTCAAAGCTGGTCTGACTAAGGCAGACTTTGATGCCACAGTGGGTGTTCA
TCCAACGGCAGCAGAGGAGTTTGTCACATTGAGGACGCCTACTCGGAAGATCAGAA
AGAGCCCTCCGTCTGAGGTTTGTCAAGGACTAGCAGCATTGTTGAATTTTATACTGG
AATGA
>orange1.1t03206
(SEQ ID NO: 12)
ATGGCAAGGAAGATGCTTAACGATGGCGAGCTGAGCCAGCCCAATCAAGAAGAACA
AACTCATTTTGACTTCGACTTGTTTGTTATTGGAGCTGGAAGTGGTGGTGTTCGTGCT
GCTCGATTTTCGGCTAATTTTGGAGCCAAGGTTGGGATATGCGAGCTTCCTTTTCATC
CGATCAGCTCTGAAGTAATTGGGGGAGTTGGTGGAACCTGTGTCATTCGTGGTTGTG
TACCCAAAAAGATATTGGTCTATGGAGCATCATTTGGAGGTGAACTTGAGGATGCA
AGGAGTTATGGATGGGAGGTACACGAGAAAATTGACTTCGACTGGAAAAAACTTCT
GCAAAAGAAGACAGATGAGATACTCAGATTAAATGGAATTTACAAGCGATTATTAT
CTAATGCTGGGGTAAAATTATATGAAGGGGAGGGAAAGATAGTTGGTCCAAATGAA
GTTGAGGTCACACAACTAGATGGCACTAAGTTAAGTTACTCAGCAAAGCACATATTG
ATCGCAACTGGAAGTAGGGCTCAACGTGCTCCTATTCCTGGCCAGGAGCTGGCTATA
ACCTCTGACGAGGCATTGAGCTTGGAAGAATTACCCAAGCGTGCTGTGGTGCTTGGT
GGAGGGTACATTGCTGTTGAGTTTGCTTCCATATGGCGAGGGATGGGTTCTACTGTG
GATCTATTATTTAGAAAGGAACTTCCATTAAGAGGGTTTGATGATGAAATGCGGGCA
GTAGTTGCCAGAAATCTGGAAGGGAGGGGAATTAATTTGCACCCGAGGACTACCAT
AAAGGAGTTGATTAAGTCAGAGGAGGGTGTAAAAGTTATTACAGATCATGGTGAAG
AGATTGTTGCAGATGTTGTACTCTTTGCCACTGGCAGGGCACCTAATACAAAAAGGT
TGAATTTGAAAGCTGTTGGTGTGGAAGTTGATCAAACAGGAGCTGTGAAGGTGGAC
GAGAACTCACGCACAAACGTACCTAGTATATGGGCTGTTGGTGATGTAACTAATCGA
ATGAATCTTACCCCTGTCGCCTTGATGGAGGGAATGTGCTTTGCAAAAACTGTTTTT
GGTGGACAACCCTGCAAACCAGACTACAGGGATGTACCCTGTGCTGTATTTTGCATA
CCTCCTCTCTCTGTAGTGGGTCTCAGTGAGGAGCAGGCCATAGAGCAAGGAAAGGG
TGATATCCTTGTTTTCACATCAAGCTTCAATCCCATGAGGAACACTATCTCTGGACG
GCAAGAGAAGACAGTAATGAAGCTAGTGGTGGATGCCGAGACGGAAAAAGTTCTTG
GAGCATCAATGTGTGGGCCAGAAGCACCTGAGATAATGCAGGGTATTGCTGTTGCA
CTGAAATGTGGAGCTACGAAGGCACAATTTGACAGCACGGTGGGAATACATCCTTC
CTCTGCTGAGGAATTCGTGACCATGCGGTCTGTGACGAGACGTGTTGCTGCCGCAAG
CAGCAATCCAAAGACAAATCTGTAA
glutathione peroxidase (GPX) genes
>Cs5g03830
(SEQ ID NO: 13)
ATGCTCCGCTGTTGTGCAAGTCGTTACCTTCTTAAACGAAATCTCGGAATCGCAACT
TCCCTTATTTTGACAAGACATTTCACTTCGAATTGTAAGCAAACCCTGTTGAGACCTT
CGAAAAGTAATCCAATTTCTTTGGTTTCAAGGCCTTGTTTCTTCGCTTCTAGATCGGA
TCATACAATGGCAAGCCAATCGAAAACTTCAGTTCACGATTTCAGTGTTAAGGACGC
CAAGGGACAGGATGTTGATCTGAGCATCTACAAGGGAAAGCTTCTTTTGATTGTCAA
TGTTGCATCACAGTGTGGCTTGACCAATTCAAACTACACTGAGCTGAGTCAGTTGTA
CGACAAGTACAAGAATCAAGGCTTGGAGATTCTGGCCTTTCCTTGTAATCAGTTTGG
AGCTCAAGAGCCAGGAGACAATGAACAGATTCAAGAATTTGCATGCACTCGCTTTA
AGGCTGAGTTTCCCATATTTGATAAGGTTGATGTTAATGGTGATAATGCTGCTCCTTT
ATACAAGCATCTGAAATCCAGCAAAGGTGGACTTTTTGGAGACAGCATCAAGTGGA
ACTTTTCCAAGTTCTTGGTGGATAAGGAGGGGAATGTTGTTGAGCGTTATGCCCCCA
CCACTAGCCCTCTGAGCATTGAGAAGGATATAAAGAAACTTCTGGAGACTGCTTAA
>Cs5g04030
(SEQ ID NO: 14)
ATGACAAGCCAATTTATACAAAACCCGGAGTCAATTTTTGACTTATCTGTCAAGGAT
GCCAGAGGCCATGAGGTAGATCTCAGCACATACAAAGGCAAAGTCTTACTCATAGT
TAATGTTGCTTCAAAATGTGGGATGACCAATTCGAATTACATAGAGCTAAGTCAGTT
ATATGACAAGTACAAAGATCAAGGCTTAGAGATTCTGGCATTTCCATGCAATCAATT
TGGTGAAGAGGAACCAGGAAGTAATGATCAGATCGCAGATTTTGTCTGCACTCGCTT
CAAATCAGAGTTTCCAATTTTTGAAAAGATTGATGTAAATGGTGAACATGCTTCTCC
ACTTTATAAGCTCTTGAAGTCGGGTAAATGGGGTATTTTTGGGGATGATATTCAGTG
GAACTTCGCCAAGTTCCTAGTTGATAAGAATGGACAAGTTGTTGATCGTTACTACCC
CACAACTTCTCTGCTTAGCCTTGAGCATGACATCAAGAAGCTCTTGGGCCTCTCCTG
A
>Cs5g31640
(SEQ ID NO: 15)
ATGGGTGCTTCAGAATCAGTCCCACAGAAATCAATCTACGAGTTCACTGTCAAGGAT
AGCAAGGGCAAGGACGTGGACCTTAGCATTTACAAAGGCAAGGTTCTTTTGATTGTT
AACGTTGCCTCCAAATGTGGTTTTACGGATTCAAATTACTCCCAGTTGACTGATCTTT
ATAACAAATACAAGCATAAAGGTTTGGAGATCTTGGCATTTCCGTGCAACCAATTTT
TGAAGCAAGAGCCTGGGACAAGTCAGGAGGCTCACGAATTTGCATGTACTAGATAC
AAGGCTGAATATCCAATCTTTCAAAAGGTACGCGTCAATGGTCCAAATGCGGAACCT
CTATATAAATTTCTCAAAGCAAGTAAAACTGGATACTTTGGGTCCAGAATAAAGTGG
AACTTCACCAAGTTTTTAGTTGATACAGAAGGAAATGTCATCGGGCGTTATAGCCCA
ACCACTTCTCCGATGGCAATTGAGGGTGACATAAAGAATGCACTGGGAGATGTTTA
G
>Cs7g06520
(SEQ ID NO: 16)
ATGGCTTCTTATTCGATGCCTTTCTCTGCAGCTTTCTCCTCACCTTTACGGCATTTCAC
ACAAAACAAAACATGCCCTGCTGCATGGGCTTCATCAATGGCGGCTTCATGGACTCC
TACTAATTCAATCAAATCATCAATTGGGTCCGCTAAACCAGGCTTTCTTCAACATGG
GCTGTTTTCCCAATCTTCAAATTTGCCTGGGTTTTTCGTGAAACGTCGTTCCTTTGGT
GTTCACGCTACAGCTGCTACTGAGAAGTCTTTATATGACTTCACCGTCAAGGATATT
GATGGTAAAGATGTTCCTCTTAGCAAGTTTAAGGGCAAAGTTCTGTTGATTGTGAAT
GTCGCTTCAAGATGTGGCTTGACACCATCTAATTACTCGGAGCTCTCTCACTTATATG
AGAAGTACAAGACTCAAGGATTTGAGATTCTAGCTTTCCCTTGCAATCAATTTGGTG
GGCAAGAGCCTGGATCAAACCCTGAGATCAAGGAATTCGCTTGTACTAGGTTTAAG
GCAGAATTTCCCATATTTGACAAGGTTGATGTAAATGGACCAAACACAGCTCCAGTT
TACCAGTTTTTGAAGTCAAGTGCTGGGGGATTTTTAGGCGACCTTGTCAAGTGGAAC
TTTGAGAAGTTCTTAGTGGACAAAAATGGTAAAGTCATCGAGAGATATCCACCAACT
ACCTCGCCTTTCCAAATTGAGAAGGACATCCAGAAGCTTGTTGTGGCATGA
>orange1.1t03689
(SEQ ID NO: 17)
ATGACATTGCGTTTGATGAAAAATAGCAACTGGGTTTCATTCCTTTTTATTGTTTTCG
CGTTTTTCTTGTATTTTTACAAATACCCATCTTCTTTTTCTGCAAAGAATATGGCTAC
GCAAGAGGCTCCCAAGTCCATCTATGACTTCACTGTCAAGGATATTCGTGGAAATGA
TGTGAGTCTAAGTGGGTACCGTGGGAAGGTTCTTCTGGTAGTGAATGTTGCTTCCAA
ATGTGGCTTAACGCAATCTAACTATAAGGAACTGAATGTTTTGTATGAAAAATACAA
AAACCAAGAGTTTGAGATCCTTGCGTTCCCTTGCAACCAGTTTGCAGGACAGGAGCC
AGGAAGCAATGAGGAAATTCAGGAAGTTGCATGCACAATGTTCAAAGCTGAATTCC
CAATCTTTGATAAGATTGATGTGAATGGAAAGAATGCGGCACCTATTTACAAGTTTC
TAAAATCAGAGAAAGGGGGATTCCTTGGAGATGCAATCAAGTGGAATTTTACCAAG
TTTCTGGTTAATAAAGAAGGGAAGGTTGTAGAGAGATATGCTCCTACAACATCACCT
CTTAAGATCGAGAAAGACATACAAAATCTGCTGGGATCATCTTGA
Catalase genes
>Cs3g27280
(SEQ ID NO: 18)
ATGGATCCCTACAAATTGCTTCCGTCAACTACTCACAATGCCCCCTTTATGACTACA
AATGCCGGCGCTCCTGTCTGGAACGACAATCACTCCTTGACTGTTGGAAGTAGAGGT
CCTGTCCTTCTTGAGGATTATCATCTGGTGGAGAAACTAGCTCATTTTGCCAGGGAG
AGGATTCCAGAACGTGTCGTCCATGCTAGGGGAGCCAGTGCTAAGGGTTTCTTTGAG
TGCACCCATGATATTTCTCACCTTACTTGTGCTGATCTTTTTCGAGCTCCAGGGGTTC
AGACACCTGTCATTGTTCGCTTCTCCACTGTTATCCATGAGCGGGGCAGCCCTGAGA
CACTCAGGGATCCTCGAGGATTTGCAGTCAAGTTTTACACCAGAGAGGGTAATTGGG
ATCTTCTGGGTAACAGTATCCCTGTTTTTTTCATCCGTGATGCAATCAAATTCCCAGA
TGTGATCCATGCTTTTAAACCTAATCCAAAGTCTCACATCCAAGAGTACTGGAGGAT
CCTCGACTTCTGCTCCCACCTTCCTGAAAGCTTGTCCACATTTTCCTGGTTCTTTGAT
GATGTGGGTATTCCACAAGATTACAGGCACATGGAAGGCTTCGGTGTTCAAACTTTT
ACACTGGTCAATAAGAATGGGAAAGTGCACTATGTGAAATTCCACTGGAAACCAAC
TTGTGGAGTTAAGTGTTTGATAGATGATGAAGAAGCAGTAAAGGTTGGTGGATCTA
ATCACAGCCACGCTACTCAGGATCTCTACGACTCAATTGCAGCTGGAAACTATCCTG
AGTGGAAACTCTACATTCAGACTATTGATCCCGATCACGAGGACCAATTTGACTTTG
ACCCACTGGACGTAACCAAGTGGTGGCCTGAGGACATCATCCCTCTGCAGCCAGTTG
GACGCTTGGTCTTGAACAAGAACATCGATAATTTCTTTGCGGAAAATGAAATGCTTG
CATTTAACCCTGGTATCGTGGTTCCTGGGATCTACTACTCAAATGATAAGATGCTCC
AATGTCGAATCTTTGCCTATGGTGATACCCAGAGGCATCGCCTTGGACCCAACTACT
TGATGCTTCCAGTTAATGCACCCAAATGTCCTCATCGCAACAATCATTATGATGGTTT
CATGAATTTCATGCACAGGGATGAGGAGGTCGATTACTTTCCTTCGAGGTATGATCC
TACTCGTCATGCTGAGAGATATCCCATTCCTTCTGATGTAATTACAGGAAGGCGTGA
GAAGGCTTGCATTGAGAAGGAAAACAACTTCAAGCAGCCTGGAGAGAGATACCGAT
CCTGGGCCCCAGACAGGCAAGAGAGGTTCCTTAATCGATGGATTAAGGCTTTATGTG
ACCCACGTGTCACCCATGAGGTTCGCAGCATATGGGTCTCTTACTGGTCTCAGGCCG
ACAGATCTCTGGGTCAGAAGCTAGCTTCTCGTCTCAATGTGAGGCCAAGCATTTGA
>Cs3g27290
(SEQ ID NO: 19)
ATGGATCCATACAAGTTCCGCCCTGCCAGTTCTTTCAACGCCCCCTTCTTTACCACCA
ATTCCGGCGCTCCGGTATGGAACAACAACTCATCAATGACGATTGGACCTAGAGGTC
CAATCCTCCTTGAGGATTATCATCTAGTGGAGAAACTTGCAAATTTTGATAGGGAGC
GGATTCCAGAACGTGTTGTCCATGCCAGGGGAGCCAGTGCTAAGGGTTTCTTTGAGG
TCACCCATGATGTTTCTAACCTTACTTGTGCTGATTTCCTTCGAGCCCCAGGGGTTCA
GACGCCTGTCATTGTCCGCTTCTCCACTGTTATCCATGAGCGTGGCAGCCCTGAAAC
CCTGAGAGATCCTCGAGGATTTGCGGTCAAGTTTTACACCAGAGAGGGTAATTTTGA
TCTTGTGGGAAACAATTTCCCCGTCTTTTTTGTCCGTGATGGAATGAAATTCCCTGAC
ATGGTCCATGCGCTTAAACCTAACCCAAAGTCTCACATCCAAGAGAACTGGAGGATT
GTTGACTTCTTCTCCCATCATCCTGAAAGCTTGCACATGTTCTCCTTCTTGTTTGATG
ATGTGGGTGTCCCACGAGATTACAGGCACATGGAAGGATCTGGTGTTAACACTTACA
TGCTGATCAATAAGGCTGGGAAAGCACATTATGTGAAATTTCATTGGAAACCCACTT
GTGGGGTTAAGTGTTTATTGGAAGATGAGGCAATCAAGGTTGGAGGAGCCAACCAC
AGCCATGCTACTCAAGATCTTTATGACTCAATTGCATCTGGAAACTATCCTGAGTGG
AAACTCTACATCCAGATTATGGATCCCGACCATGAGGACAAATTTGACTTTGACCCA
CTTGATGTAACCAAGACCTGGCCTGAGGACATCTTGCCCTTGCAACCTGTGGGCCGG
TTGGTTTTGAACAAGAACATTGATAACTTCTTTGCAGAGAATGAACAGCTTGCTTTC
TGCCCTGCTATTGTGGTTCCTGGCATCTACTATTCAAATGATAAGTTGCTCCAGACTC
GAATCTTCTCCTATGCTGATACTCAGAGGCACCGTCTAGGGCCGAACTATCTTCAAC
TCCCAGTAAATGCACCCAAATGTGCTCAGCACAACAATCATTATGACGGTTTCATGA
ATTTCATGCACAGGGATGAGGAGATCGATTACTTCCCTTCAAGGTATGATCCTGTCC
GTCATGCTGAGATGCATCCAACTCCTCCTGAAATCTGCGGCGGAAGGCGCGAGAAG
TGCATAATTCCAAAGGAAAACAACTTCAAACAACCTGGAGAGAGATACCGATCTTG
GGCACCAGACAGGCAAGAGCGATTCATCTGCAGATGGGTTGATGCTTTATCTGATCC
ACGCGTCACCCATGAGATCCGCCACATATGGATCTCATACTGGTCTCAGGCTTGCAA
GTCTCTGGGTCAGAAGTTGGCATCTCGTCTGAATGTGAGGCCTAACATTTAA
Ascorbate peroxidase (APX) genes
>Cs1g25730
(SEQ ID NO: 20)
ATGGTGGGAAGCATCGCAATTGATGCAGAGTACTTGAAGGAAATCGAGAAGGCTCG
TCGAGACCTTCGCAGTCTCATCTCCAGCAGAAGCTGCGCTCCTATCATGCTTCGCTT
GGCGTGGCACGATGCTGGAACTTATGATGCGAAGACAAGGACGGGCGGACCTGATG
GCTCCATCAGGCACGAGCAGGAGTACGCTCATAATGCTAACAATGGTTTGAAGATT
GCTATTGATTTATGTGAGGGGGTGAAGGCCAAGCATCCAAGGATTACGTATGCTGAT
CTTTACCAGCTAGCGGGAGTTGTTGCGGTTGAGGTCACTGGAGGACCTGCTATTGAC
TTCGCTCCTGGAAGAAAGGACTCGTCAGAATCTCCTGAAGAAGGGCGTCTTCCTGAT
GCCGCTCAAGGTGTATCCCATCTGAGGGACATCTTTTATCGGATGGGTCTATCTGAT
AAGGACATTGTGGCACTGTCTGGAGGCCACACACTGGGAAGAGCACATCCGGAAAG
GTCTGGTTATGAGGGACCCTGGACAAAGGAGCCTTTAAAATTCGACAACTCTTACTT
TGTGGAGCTTCTGAAAGGGGAATCAGAGGGATTGTTAAAGCTTCCCACGGACAAGG
CTCTACTGGAGGATCCTATGTTCCGCTACTACGTGGAGCTATATGCAAAGGATGAGG
ATGCATTCTTCACAGATTATGCTGCTTCACACAAGAAACTTTCAGAGCTAGGCTTCA
ATCCACCTTCCTTGGGTTTTAAGGATGCTGTGAAGAACACTGCATTGTTGGCACAAA
GTGCTGTCGGAGTCGCTGTTGCTGCAGCTGTAGTGATTTTCAGTTACTTTTATGAAGT
TCACAGAAAAACCAAGTAA
>Cs8g02730
(SEQ ID NO: 21)
ATGGCTTTACCGGTCGTTGACACAGAGTACCTGAAGGAGATCGACAAGGCTCGTCG
CGATCTCCGCGCTCTTATCGCTTACAAGAACTGTGCTCCAATCATGCTCCGTCTAGC
GTGGCACGACGCGGGAACATATGACGTGAACACGAAGACTGGTGGACCAAATGGCT
CGATAAGGAACGAAGAGGAGTACTCTCATGGCTCCAACAATGGCTTGAAAATCGCT
CTTGATTTTTGTGAGGAAGTGAAGGCTAAACATCCAAAAATAACATATGCAGACCTA
TACCAGCTTGCGGGTGTTGTTGCAGTTGAGGTCACTGGAGGTCCTACAGTCGACTTT
GTTCCTGGTAGAAAGGATTCAAAAATTTCTCCCAAGGAAGGGCGTCTTCCAGATGCC
AAACGAGGTGCACCTCATCTGAGGGATATCTTTTATCGGATGGGTCTGTCTGACAAG
GATATAGTGGCACTCTCAGGGGGCCATACTTTGGGAAGGGCACATCCAGAGAGATC
AGGTTTTGACGGCCCTTGGACCAGGGAGCCTCTGAAATTTGATAACTCATACTTTGT
GGAGTTGCTGAATGGGGAATCAGAAGGATTACTGCAACTTCCAACAGACAAGGCTT
TATTAGAAGACCCTGAGTTCCGTCGTTATGTTGAGTTGTATGCAAAGGATGAGGATG
CATTCTTTAGAGATTATGCAGCATCACATAAGAAACTTTCAGAGCTAGGGTTTTCAC
CCGGTTCTGGCTCCAAATCAACAGCAAATGATTGCACCATATTAGCTCAAGGTGCAG
TTGGAGTAGCAGTTACTGTGGCAGTGGTGATTCTAAGCTACTTTTACGAAGTTCGCA
AAAGGATGAAGTGA
>Cs3g17910
(SEQ ID NO: 22)
ATGGGCGCTTCTTTTCTCTCTACGGTTCCTTTTATGCTTCCCATTGTTCCTCCCCCATC
GCGCAGCGCTTTCTCTACTGTGGCCACCAGATGCTCCGCTCATGCTGGTGCTATACG
TTGTCATAAAATTGAGACTCAAATTACTGTTGAAGCTGGTTTTGGTAGAAGGGATAT
TCTGAAATGCATTGGTGCGACCATTGGCATGGAAATAATATCAAGCTCAGGATCAGT
TGAAATGGCACGTGCTGCTGATTTGATACAACGCAGACAGCGTTCGGAGTTTATTTC
CAATATCAAGGAGACTCTCCAAACTGCTCTCAAGGGAAATCCAGATCTTATCCCATC
TCTACTAACTTTGGCACTCAATGATGCCATGACTTATGATAAGGCTACAAAATCTGG
GGGCCCTAATGGATCCATAAGGTTCAGCTCAGAGATAAGCAGACCAGAAAATAAGG
GTCTTTCTGCTGCCATGAATTTGTTAGAGGAAGCAAAGAAGGACATAGATTCATATT
CTAAGGGTGGACCAATATCCTTTGCCGATCTCATCCAGTATGCAGCACAAAGTGCCG
TTAAGACAACATTTTTGGCATCAGCCATCCGCAAATGTGGAGGGAATGAAGAGAAA
GGGCGCTTATTGTACACTGCATATGGCTCAAATGGTCAGTGGGGCCTGTTTGAAAAG
CAATTTGGAAGGACAGATGCTCAGGAGCCGGATCCAGAGGGGAGGGTTCCCCAGTG
GGAGAAAGCTACTGTCCAGGAAATGAAAGATAAATTCTCAGCAATTGGCCTTGGTC
CCCGCCAGCTAGCTGTTATGTCTGCGTTCTTGGGTCCTGATCAGGCAGCAACAGAAG
CCTTATTGGCCACTGATAAAGATGTTTCTCCTTGGGTTCAGAAATACCAACGTAGCA
GAGAAACAGTGTCTCAGACTGATTACGAGGTCGATCTGATAACTACATTCACGAAAT
TAAGTAGCCTGGGCCAACAAATCAATTACGAGGCTTACACATATCCTGCTCAAAAG
ATTGAATTGAGCAAACTCAAATTGTAG
>Cs3g19810
(SEQ ID NO: 23)
ATGGCAGCTCGGCCAGCGTCACTGACACAACAACAGATACGACTGCAATCAAAATC
ACAATCATCGATGGCTTCCTCTTTAAGCACCGCCGCTTCTTCTCGCCTTCTTTGCTCC
ACCACCGCAGCCGCCGCCGCAGCTGCTAAGCTTTCTTTCTCTTCCGCTTCTTCACTCT
CTTTCTCTCTCTCCTCTCCTTCCTCCCTCAAATGCCTCAGATTCTCTCCTCTCATTAGC
CAGAGGAGATCGTCTGTGAACAGAGGATATAGCACCGTAGCTACGACGAAATGTGC
GGCGTCGGATCCTGATCAGTTGAAGAGTGCTAGAGAAGATATTAGGGAGCTTCTCA
AGTCTACATTTTGCCATCCTATTTTGGTTCGTCTAGGATGGCATGATGCTGGTACTTA
TGACAAGAACATTGAGGAGTGGCCTCGAAGGGGAGGAGCTAATGCAAGTCTTAGAT
TTGAGGTTGAGCTAAAACACGCAGCCAATGCTGGGCTTGTGAATGCATTGAAACTG
ATTCAACCTATCAAGGACAAATATTCCGGTGTCACATATGCAGACTTATTCCAGTTG
GCTAGCGCTACTGCTATAGAGGAAGCCGGGGGCCCCAAGATCCCAATGAAGTATGG
AAGAGTGGATGTCTCAGGACCTGAGCAGTGCCCTGAAGAAGGAAGGCTTCCCGCTG
CTGGGCCTCCATCACCTGCTGAACATTTAAGAAATGTCTTTTATAGAATGGGATTAA
ATGACAAGGAAATAGTTGCCTTATCTGGTGCACACACAGTGGGAAGGTCTAGACCT
GAACGAAGTGGTTGGGGCAAGCCAGAAACAAAGTACACGAAAGATGGACCAGGAG
CACCTGGTGGGCAGTCCTGGACAGTACAATGGTTGAAATTTGACAACTCCTACTTCA
AGGATATTAAAGAAAGAAGGGACGAAGATTTACTCGTGCTGCCTACTGATGCTGTTC
TTTTTGAAGATCCTTCATTCAAGGTTTATGCTGAGAAATATGCTGAGGATCAGGAAG
CATTCTTTAAGGATTATGCTGAAGCCCATGCCAAACTCAGCAACCTTGGGGCAAAAT
TTGACCCTCCTGAGGGTATTGTGCTAGATGATGGTGCTGCTCCAGAGAAGTTTGTAG
CAGCCAAGTATTCGTCCGGAAAGGACTGA
>Cs6g04140
(SEQ ID NO: 24)
ATGGGAAAGTGTTATCCAAAAGTGAGCGATGAGTATCAGAAAGCAGTGGAGAAATG
CAAGAGGAAACTCAGAGGACTCATTGCTGAGAAACACTGTGCCCCTATCATTCTTCG
CTTGGCATGGCACTCAGCGGGGACTTATGACGTGAACACGAAGACAGGAGGGCCTT
TTGGGACGATCAGGCACCCAGATGAGCTTGCTCATGAGGCTAACAATGGTCTTGATA
TTGCTGTCAGGCTCTTGGAGCCCATCAAGCAGCAGTTTCCTATCTTGTCCTACGCTGA
TTTCTATCAGTTGGCTGGAGTTGTTGCCGTTGAAGTTACCGGAGGGCCTGAAATACC
TTTCCACCCTGGAAGACCGGACAAATCTGATCCGCCCCCAGAAGGTCGCTTGCCAAA
TGCCACCAAAGGTTCTGACCATTTGAGAGATGTCTTCGGCCACATGGGTCTGAGTGA
TAAGGATATTGTTGTTTTATCCGGCGGTCATACATTGGGTAGGTGCCACAAGGAGCG
TTCTGGATTTGAAGGACCCTGGACTAACAATCCACTTATTTTTGACAATTCATATTTT
AAGGAATTGCTGAGTGGAGAGAAAGAAGGTCTACTTCAGCTGCCATCAGACAAGGC
TCTTCTCGAGGACCCAGTGTTTCGTCCTCTTGTTGAAAAATATGCTGCAGATGAGGA
TGCCGTCTTTGCAGATTACGCTGAAGCTCATTTGAAGCTCTCAGAACTCGGATTTGC
GGATGCTGAGTAA
>Cs7g05430
(SEQ ID NO: 25)
ATGAGTTCATCAACTGCCTCCAGCCTCTGCGGCTGCTCCACCGCTTACTACTCTTCCC
CTCTCCTCGCTCCCAAATTTAAATTCAAGTTTCCCGCCAAATCACAACGGTCCTCTCT
CTCAACGGTCGAATTTCGCGCCAATCCCCTCACAATTTCTTCCTCCTTAGTCAACCAC
CGAGGGAAAGATGGATCTTGCAATTCGAATAGGAGGAGGAGAGGACTGTTGCTTAC
GGCTACACTTCCATTTCTTCTTCCTCTCCACGAATTTGTGCAAGACCTGGGCGCAAAA
GCTGAGTCAGGTATGAGTGACTATCTGCTTATGAAAGAAGAAGTGACTAAGGTAGT
TTCAAAAGGCAAGGCTGCTAGTGTACTTCGTCTTGCATTTCATGATGCAGGGACCTT
TGAAATGGATGATAATTCGGGTGGTATGAACGGTTCTATAGTTTATGAACTGGAAAG
ACCTGAAAATGCAGGCCTGAACAAGCCTTTGAAGATTCTAGAGAAAGCAAAGGGTG
ACGTGAATGCAATACGGCCAGTGTCATGGGCAGATATGATCGCTTTGGGTGGAGCT
GTAGCAGTTTCAGTTTGCGGAGGTCCAAACATCCCAGTTCCTATGGGCAGACTTGAT
TCAATGGAGCCTGATCCTGAAGGAAAACTTCCGCAAGAAACTTTGGATGCTTCTGGT
TTGAAGCAATGCTTTCAAAGAAAAGGCTTCTCAGCACAAGAGCTAGTTGCTTTATCT
GGAGCCCATACTCTTGGAACCAAAGGTTTTGGAAATCCAATAGTTTTCGACAATTCG
TACTATAAAATTCTTTTAGAGAAGCCATGGCAGTCTTCAGCTGGTATGTCAAGCATG
ATTGGACTTCCTTCAGATCGTGCACTTGTTGAGGATGATGAATGTTTGAGATGGATC
AAGATGTATGCTGACAATCAGAATATGTTCTTTGAAGACTTCAAGAATGCTTACGTT
AAACTAGTGAACTCCGGAGCAAGGTGGAGAAGCTTGTGA
>Cs7g05450
(SEQ ID NO: 26)
ATGCATGAGCATGACGTGGCACGAGGGGTAAATGAGGTAAAGAACAAGGGTAAAA
GTGGAAGTGAAAAAAAGGGACCCACGGGTGACCAAGTAAGCAAGAAAGGTGGTAT
GACAGGGGGATTTTATGATTACAGACGTTCAAAATTGAAGTTTATGAATATAATTAT
GCGGAACACAAAAACTATCAATGCCAGGGAAGAAGAAGTGGAGAAAGTTCGATAT
AAGCTCCAAGAACTAGACCAAAAGATTACAGAACATGTTACTGAGCAGCAGAAAAC
TGATGCCAAGCGAGCTCATGATAAATCAGAACTGGAACAGCTTAAGCAGGATATCG
CTAATGCTAATAAGCAGAAGCAAATTATTTCTAAAGCTCTTGAAAATAAGGAAAAG
TCACTTGCAGATGTGAGGACACAACTTGATCAACTTGAAGCTAGTATGGCTATGAAA
CAGGCTGAAATGAACACAGACCTCATTGATCACTTATCACTAGATGAAAAAAATCTT
CTATCGCGTTTAAACCCTGAAATAACTGAGCTCAAGGAGAAGCTTATCACCTGCAGG
ACAGATCGCATTGAGTATGAAACCAGAAAAGCGGAGCTTGAGACTAATCTAACAAC
TAACCTTATGAGGCGAAAGCAAGAGTTGGAAGCTCTTATATCTTCTGCAGAGAATGA
TGTGATGCTAAGTGAAGCTGAATCAAAGAAGCAGGAATTGGCAGATGCTAAATCAT
TTGTTGAAGATGCAAGACAAGAGCTTAAAAGGACCTTTGAAATGGATGATAATTCG
GGTGGTATGAACGGTTCTATAGTTTATGAACTGGAAAGACCTGAAAATGCAGGCCT
GAACAAGCCTTTGAAGGCATGCTCTATTCTAGAGAAAGCAAAGGGTGACGTGAATG
CAATACGGCCAGTGTCATGGGCAGATATGATCGCTTTGGGTGGAGCTGTAGCAGTTT
CAGTTTGCGGAGGTCCAAACATCCCAGTTCCTATGGGCAGACTTGATTCAATGGAGC
CTGATCCTGAAGGAAAACTTCCGCAAGAAACTTTGGATGCTTCTGGTTTGAAGCAAT
GCTTTCAAAGAAAAGGCTTCTCAGCACAAGAGCTAGTTGCTTTATCTGGAGCCCATA
CTCTTGGAACCAAAGGTTTTGGAAATCCAATAGTTTTCGACAATTCGTACTATAAAA
TTCTTTTAGAGAAGCCATGGCAGTCTTCAGCTGGTATGTCAAGCATGATTGGACTTC
CTTCAGATCGTGCACTTGTTGAGGATGATGAATGTTTGAGGTTTGTCATCTTCGAAG
AGCCTCTTAAAACTTACCTGAATTCTATCAACTTCTTTTAA
>Cs8g17370
(SEQ ID NO: 27)
ATGACGAAGAATTACCCCACTGTTAGCGAGGATTACAAGAAGGCCGTGGAGAAATG
CAAGAGGAAGCTCAGAGGCTTCATCGCTGAGAAGAACTGCGCTCCACTCATGCTCC
GTATCGCGTGGCACTCAGCTGGTACATATGATGTGAAGACCAAGACCGGAGGTCCA
TTCGGAACCATGAGGCTCGCTGCTGAGCAGGCACACTCAGCCAACAACGGCCTCGA
TATTGCCGTTAGGCTTTTGGAGCCGTTCAAGGAGCAGTTCCCTACCATCTCCTACGCT
GACCTTTATCAGCTTGCTGGTGTTGTTGGCGTTGAGGTTACCGGTGGGCCCGATATC
CCCTTCCACCCTGGAAGAGATGACAAGGCTGAGCCACCTCAAGAAGGCCGGCTTCC
TGATGCTAAACAGGGTAATGATCACTTGAGGCAAGTCTTTGGTGCTCAGATGGGCCT
CAGCGACAAGGATATTGTTGCTCTCTCTGGTGGCCACACCCTGGGAAGGTGCCACAA
GGAGAGGTCTGGATTTGAGGGACCATGGACCCGCAACCCTCTCATTTTTGACAATTC
CTACTTCACGGAGCTCTTGACTGGTGAGAAGGATGGTCTTCTGCAATTGCCATCCGA
CAAGGCCCTTCTGGATGACCCTGTTTTCCGCCCTCTTGTTGAGAAATATGCTGCGGAT
GAGGACGCTTTCTTCGCTGATTATGCTGAGGCTCATCTCAAGCTCTCTGAGCTAGGA
TTTGCTGAAGCCTAA
glutathione S-transferase genes
>Cs7g14300
(SEQ ID NO: 28)
ATGAAGCCAGAGCTGAGAACTTGTTTGGGGGCAAGAGTGACCTGCTGCTCAAATCA
AACCCCATCTACTAGAAAGTCCCAGTTTTTCTACACGATGGGAAACCATTGTGTGAA
TCCACTATTATATTTAACTAAATTGACGAGACTTGGCCTTCGCCACCACTTCTCCCAT
CCTGTGCTTACGAGAGAGCTAAGACAAGGTTCTGGGCAGATTTTATTGACAAGAAG
GTCAGTCTATGATTCTCAAGTATTCGATGCAGTCTGCAACATATGGAAAAGCAAGGG
CGAGGTCCCCGAGACGGCGAAGAATGAATTTATAGAGATTTTGAAGCAACTTGAGG
GAGCTTTGGGGGAGAAGGATCTCTTTGGCGGGGATAGCTTTGGATTCGTTGATGTCA
TTGCCATTCCACTGACTTGTTGGTTTTACGCGGTTGAGAAATTTGGGGGATTTAAGGT
TGAGAATGAGTGCCCCAAATTCTCAGCTTGGATGAACAAGTGCATGCAAAGAGAAA
CTGTTGCCAGGATGCTTCCAGACCCAGAAAAGGTTACGAATTTGTTATCACGTTGA
>orange1.1t03605
(SEQ ID NO: 29)
ATGGGTGAAGTGAATCTGCACGGAGAATGGTCAAGTCCATTTAGTTACAGAGTAAT
ATGGGCTCTGAAACTAAAGGCCATAGAATTTGAATATATCGAAGAAGATCTTTCCAA
CAAGAGCCCTTTGCTTTTGCAATATAACCCAATTTACAAGAAAATCCCAGTTCTTGTT
CATGGAGGAAAACCAATCAGTGAGTCCATGATTATCCTCGAATATATTGAAGAGAC
ATGGCCAAAAAATCCATTGCTGCTGCCCAGTGATTCTTTTGAAAGAGCCATGGCTCG
CTTTTGGATCAAATTTTCCGAGGAAAAGGGTCCTACAATTTGGAAGATATTTGTGAC
AAGTGGAGAAGAACAAGAAAAGGTAAAAAATGAAAGCTTGGAAATTCTAAGAACC
ATAGAAGAGAAGGGCCTTGGAGAGAAGAAATTTTTCGGAGGAGAGAACATTGACCT
TCTAGATCTAGCCTTTGGTTGGATGCCCTATTGGCTGGGAGTCATTGAAGAAATATT
AGGGCTCAAGTTACTTGAAGCTGACAAGTTCCCTCGTTTGCATGCATGGATAAAAAA
TTTCAAGCAGGTTTCAATTATCAAAGATAACCCTCCCGACCGTGATCGGATGCTCAT
TCATTTCAGCAGTCTGAGGGAGAAGTTACTACATTAA
>Cs1g08610
(SEQ ID NO: 30)
ATGGCTGAGAATGTGGTTTTATTGGGCTTTTGGCCAAGTTCTTTTGCAATGAGAGTG
AAAATTGCACTGGCCGAGAAGGGAATCAAATATGAATCAAAAGAAGAGAACTTGTT
CGATAAGAGCCCTTTACTCCTTGAGATGAATCCGGTTGACAAGAAAATTCCTGTTCT
AATTCATGACGGGAAACCCATTTGTGAATCCCTTATTATCATTCAATACATTGATGA
AGTTTGGCATCACAAATCTCCTCTTATGCCTTCTGATTCTTACCTTCGATCTCAAGCC
AGGTTTTGGGCTGATTATGTTGACAAAAATATATACGGTATTGGGAAGAGGATTTGG
TCGGGGAAAGGAGAAGATCAAGAGGCAGCAAAGAAGGAGATGATAGGGTTTTTGA
AGAACATGGAAGCAGAGCTCGGAGACAAACACTTCTTCGGCGGCGAGAGTATTGGA
TTTGTAGATGTGGCGTTGGTTCCATTCACAACATGGTTTTACACGTACGAGACTTTTG
GGAACTTGAGCGTTGAAGCAGAGTGCCCCAAGCTTATTGCGTGGGCCAAGAGGTGC
TCACAGAAGGAGAGTGTGTCTGCCTCCCTTGCTGATCCTCACGACATCTACGACTTT
GCTTTGGGCCTTAGAATGAAATATGGGATTCAGTAG
>Cs7g15760
(SEQ ID NO: 31)
ATGGCAGAAGAAGTGAAGCTTTTGAAGACGTGGTCAAGCCCCTTTGGTTTGAGAGCT
TTTTGGATATTGAAACTCAAGGGCGTTCAATTTGATTTCATAGATGAAGATCTCTCTA
ACAAAAGCCCTTTACTTCTTCAATCTAATCCTGTCTATAAAAAAGTTCCTGTACTCAT
ACACAACGGGAAACCAATCTCAGAATCACTAGTAATTCTTGAATATGTTGATGAGAC
TTGGAAGCAAAATCCTTTGCTTCCTGAAGATCCTTACGAAAGAGCCAGGGCTCGCTT
CTGGGCTAAATTTGGTGAAGATAAGGTTTTGGTATCAATATGGAATGCTTTCATTAA
GCAAGGGAAAGAACAAGAGGAAGCTATTGGCCTAGCAATTGAGACTCTGAAATTCT
TAGAAGAAGAGCTGAAGGGAAAGAGATTTTTTGGTGGAGAGAAGATTGGATTGGCA
GATCTTGCATTGGGCTGGCTTGCTAATTTGATCGGCGTATTTGAAGAGGTAATTGGT
GTGAAACTGATAGAAAAGGAGCGGTTTCCACTGCTGTCAGCTTGGATGCAAGAGTTT
GCTGAGGCCCCTGTAATCAAAGAAAGCTGGCCACCTCATGAAAAACTAGTCACCAA
ATTTCGTGCCATCCGAGAGCCTTATGTTGCAGCTGCTGCCCAGCCTAAATGA
>orange1.1t03610
(SEQ ID NO: 32)
ATGGCTGAAGTGAAGCTTCATGGGGCATGGGCGAGTGCATTTTGTTACAGAGTGATA
TGGGCTCTGAAATTGAAAGGCATAGCATATGATTACATTGAAGAAGATCTCTCTAAC
AAAAGTGCTTTGCTTTTGAAGTATAATCCAGTTCACAAGAAGGTCCCAGTGCTTGTT
CATGACGAAAAATCCGTTTGCGAGTCCATTGTTATCCTTGAATACATTGAAGAGATG
TGGCCACACAATCCCTCCTTGATGCCAAGTGATCCTTATGATAGAGCTCTGGCTCAT
TTCTGGATTAAATTTGCCGAAGACAGGGTTTGCTTCAGAACTCTAGCTAATGCGTTT
ATAAGTTCATTATTTTCATGTTTATTTGTTATTAGAGAAAATGATCAGTCCTCCATTG
TATGTCAAAATTAA
>Cs3g17620
(SEQ ID NO: 33)
ATGGCTGAAGAAAGTGTGAAGTTGCTGGGTTACTGGGCAAGCCCCTTTGCTCTTAGA
GTTAAGTGGGCGCTCAAACTGAAGGGAGTTCACTACGAATATGTTGAAGAGAATCT
CCCCAACAAGAGTCCCTTGCTGTTGCGATACAATCCGGTTTATAAGAAGATTCCAGT
TCTTGTACACAATGGGAAACCATTGGCAGAATCACTACTGATCATCGAATACATTGA
TGAAGCGTGGAAACAAAATCCTCTCCTCCCAGACGATCCTTACGAACGGGCCAATG
CACGTTTGTGGGCAAAATTCTTTGATGAGAAGTGTGTACCTGAAGTCATGGGAGCAT
TTGCCTCAAAAGGGGAGGAACAAGAGAAAGCTGCAAAAGCTCGAGAGAATCTGAA
GATGCTGGAAAGGGCTCTGGAGGGGAAGCCCTTTTTTGGTGGTGACAAGATAGGCT
TTCTGGACATTGCTGTGGGGTGGATTGGAATCTGGGGTCGAATTGTTGAGGAAATTG
CAGGTGTCAGCCTGATTGATGCAGAGACGATGCCTTTGCTTACTGCTTGGTTGAACA
ATTTTCTTGAAGTTCCTGTTATCAAAGCATGCATACCATCTTGGCACGAATTGCTTGA
GCACAATAAAGGCTTCCACAAGATCTTGACAGGTTCATCAACTTGA
>Cs7g15770
(SEQ ID NO: 34)
ATGGCAGAAGAAGTGAAGCTTTTGATGACGTGGTCAAGCCCCTTTGCTTTGAGGGTT
GTTTGGGCATTAAAGCTGAAGGGTGTTCAATTCGATTCAATAGAAGAGGATCTCTCC
AACAAAAGCCCTTTACTTCTTCAGTACAATCCTGTTCACAAAAAGATTCCTGTGCTT
GTACACAACGGGAAACCGATCTCAGAATCTCTCGTAATTCTTGAATATGTCGACGAG
ACTTGGAGGCAATACCCTTTGCTCCCTGATGGTCCTTATGATAGAGCCAGGGCTCGC
TTTTGGGCCAAATTCAGCGATGATAAGGTTTCTCCATCAATGTGGGGTGTTTTCATTA
AACAAGGGAAAGAACGAGAGGAAGCTTTTGATCAAGTTTTTGAGAATTTGAAATTT
TTAGAAGAAGAGTTGAAGGGAAAGAAATTTTTTGGGGGAGAGAAGATCGGGTTTGC
AGATCTTGCATTGGGTTGGATTGCTGAAAGGGTCTGTGTATTGGAAGAGATTATTGG
TGTGAAAATTGTGGAGGAGGAGAAATTTCCACTGGTGTCAGCTTGGATGCAAGAGT
TTGTTAAGGTTCCAATCATCAAAGAAAGCTTGCCCCCTCATGAGAAACTAGTCACGA
AAATACGCGGCATCAGAGAGAAATTTATTGGAGCTGCATAG
>orange1.1t03617
(SEQ ID NO: 35)
ATGGCTGAAGTGAAGCTTCATGGGACCCTGCTGAGTCCATTTGTTTACAGGGTGATT
TGGGCTCTGAAATTGAAAGGCGTGCCATATGAATTTGTTGCAGAAGATCTCTCAAAC
AAAAGTGCTTTGCTTTTGAAGCACAATCCGGTTCACAAGAAGATCCCAGTGCTTGTC
CATGGCGGAAAACCAGTTTGTGAGTCCATGGTTATTCTCGAATACATCGAAGAGACG
TGGCCACAGAATCCCTTGATGCCAAATAATCCTTATGATAGAGCTCTTGCTCGATTC
TGGATTAAATTCGCCGAAGACAAGGGTGTTGCTGTTTGGAAAATGTTTCGTAGCACT
GGACAAGAACTACAAAACACAATGAAGGAAGCCTTGGAAATGCTACAAACCGTTGA
AGAGCATGGCTTGGGAGAGAAGAAATTTTTCAATGGAGACAATATTGGCTTAGTGG
ACCTAGCGTTTGGTTCAATTGTTTATTGGTTGCAAGTCATTGAAGATATAGTGGGCG
CTAAACTATTTGAATCACACAAATTCCCTCACTTGCATGCCTGGATTGAAACTTTCA
AGCAAGTCACTTTGATTGAAGAAAATCTTCCTAATCAGGATGAACTATTGGTTGCTC
TCAAACTTCGTCGTGAACAGTTGATGGCTTCTGCTTGA
>Cs5g03900
(SEQ ID NO: 36)
ATGGAAGGAGAAAACGAAGTTAAACTCCACGGGATGTGGGCGAGTACTTACTCTAA
GAGGGTTGAATTAGCCCTCAAAGTTAAAGGCATCCCATACGAGTACATAGAAGAAG
ATTTGAGGAACAAGAGCCCATTGCTTCTCCAATATAATCCTGTTTACAAGAAGGTCC
CTGTGCTTGTTCACAACGGAAAACCCATAGCTGAGTCATATATAATCCTGGAATATA
TTGATGAGACCTGGCAAAATGGTCCTCGACTCCTGCCTCAGGATCCCTACCAGAGAG
CTCAAATTCGATTCTGGGCCAGCTTCATTCAACAACAGTTATTTGGGTCTTTTTATAA
ATTGATTTCAAGTAATGGGGAGGAACAAGAGAAGGCAGTTGAGGAAGTTTTTGAGA
AGATGAACGTGTTGGAAAAAGGAATGAAAGAACTGCTACCAGAGGGCGTTCCTGAA
GTTGATGGCAATAATTCCGGAGTATTGGACATTTTGGTGACTGCAACATTCGGTCCC
TACAAAGCTCATGAAGAAGTTTTCGGTTTGAAGGTCTTGGATCCTGAGAAGTATCCA
TTGTTGCAAGCAGAACTAAGCAAAAAGAGAGGGATGGAAGAACAAAACAAAGTTA
AGCTACATGGGACGTGGATTAGCACTTACTCTAAGAGGGTTGAATTAGCCCTCAGAG
TTAAAGGCATTCCCTATGACTACTTAGAAGAAGATTTGAGCAACAAGAGCCCATTGC
TTTTCCAATACAATCCTGTTCACAAGAAGATCCCTGTGCTTGTTCACAATGGAAAAC
CAATACCAGAGTCGTCCATCATCCTGGAATACATCGACGAGACCTGGAAAAATGCT
CCTCGATTCCTGCCAGAAGACCCCTATCAGAGAGCTCAAATTCGATTCTGGGCTGGT
TTCATCCAACAGGTGTTTGAAGCTATGGGTAAAGCGTTCATGACATCAGGGGAAGC
ACAAGAAAAAGCAATTGAGGAAACGCATGAGAAAATCAACGTGTTAGAAGAGGGA
ATGAAAGGATTATTTTCTGAGGAAGCTCCGAAAATTGATGGCCAGAATTTCGGACTG
CTAGATATCATGGTGTGCGCAGTACTGAGTACCCACAAAGCTTCTGAAGAAGTCCTT
GGTAATGGTGTCAAGATTTTGCATCCAGAGAGACACCCCTTCTTGTCCTCATGGGTT
GAGAAGTTGAACAAGCATCCTGTAGTGAAGGAGAATACTCCTCCGTATGACAAGAT
CATCGGTTTTCTTCACTTCATAACTCACAAGACCAAAGAGTCTCGGGTCTGCTAG
>Cs7g15790
(SEQ ID NO: 37)
ATGGCGGAGGTGAAGCTTCTGAAGACATGGTCTAGTCACTTTGCTTTGAGGGTTGTT
TGGGCATTAAAGCTGAAGGGTGTTCAATTTGATTCCATAGAAGAAGATCTCTCCAAC
AAAAGCCCTTTACTTCTTCAGCACAATCCTGTTTACAAAAAGATTCCTGTGCTTGTAC
ACAACGGCAGAGCAATCTCAGAATCTCTGGTAATTCTTGAATATATTGATGAGACTT
GGAAGCAATACCCTTTGCTCCCTGATGATCCTTATGAGAGTGCCAGGGCTCGATTTT
GGGCCAAATTCAGCGATGATAAGGTTTTTCCATCAATATGGGGTGTTTTCATCAAAC
AAGGGAAAGAACAAGAGGAAGCTTTTGCTCAGGTTTTTGAGAATTTTAGTTTTTTAG
AAGAAGAGTTAAAGGGAAAGAAATTTTTTGGGGGAGAGAAGATCGGATATGCAGAT
CTTGCATTGGGTTGGATTGCTGAGAGGGTCTTTGAATTGGAAGAGATTGGTGTGAAA
GTTATAGAGAAGGAGAAATTTCCATTGGTGTCAGCATGGATGCAAGAGTTTCTTAAG
GTTCCAGTAATCAAAGAAAGCTTGCCACCTCACGAGAAACTAGTCACCAAAATGCG
TGGCATCAGAGAGAAATATCTTGGAGCTGCACATCCTGAATGA
>orange1.1t03618
(SEQ ID NO: 38)
ATGGCTGAGGTGAAACTTCATGGGCGATTGCTGAGTCCATTTGTTTGCAGAGTGATT
TGGGCTCTGAAATTGAAAGGCGTGCCATACGAGTTTGTTGCAGAAGATCTCTCAAAC
AAAAGTGATTTGCTTTTGAAGTATAATCCAGTTCACAAGAAGATCCCAGTGCTTGTC
CATGGCGGAAAACCTGTTTGTGAGTCCATGATTATTCTCGAGTACATTGAAGAGACG
TGGCCACAGAATCCCTTGATGCCAAATAATCCTTATGATAGAGCTCTTGCTCGATTTT
GGATTAAATTCGCCGAAGACAAGCTGGTTGTTGCTATTTTGAAATTGTTTCGTAGCA
TTACTGGACAAGAACTAGAGAATGCAAAGAAGGAAATCTTGGAAATCCTACAAACC
CTTGAAGAACATGGCTTGCGAGAGAGGAATTTTTTCAATGGAGACAATATTGGCTTA
GTTGACATTGCATTTGGTTCAATGCTTTATTGGATGCAAGTCATTGGAGATGTAGTG
GGAGTTAAACTGTTTGATTCACACAAATTCCCTAGCCTGCATACGTGGTTTGAAAAT
TTCAAGCAAGTCCCTGAAATTGAAGAGAACTTTCCTAATCGGGATGAACATTTGGTT
TATCTCAAACATCGCCGTGAACAGTGGGTGGCCTCTGATTGA
>Cs5g15160
(SEQ ID NO: 39)
ATGGCGGACGAAGTGATTCTATTGGATTTCTGGGCCAGTATGTTTGGGATAAGGGTC
AGGATTGCTCTGGCAGAGAAAGGCATCAAGTATGAGTACAAAGAACAGGACTTGTT
CAACAAGAGCCCGCTGCTTCTGCAGATGAATCCCGTCCATAAGAAGATCCCGGTTCT
TATCCATAATGGCAAATCAGTCTGTGAGTCATCCATCATTGTTCAGTACATTGATGA
AGTCTGGAAGGACAAGGCTCCATTGCTTCCCTACGATCCTTACCAAAGAGCTCAAGC
GAGGTTCTGGGCCGATTTTATTGACAAGAAGATGTACGATACTTCGAGGAAGACAT
GGGCCACAAAGGGAGAAGAGCAGGAGGCAGCAAAGAAGGAGTTCATCGAAGTTCT
GAAGACCTTGGGAGGGGAGCTTGGAGACAAGCCTTTTTTCGGAGGAGACAACTTTG
GGTATGTGGATTTGTGTTTGATCCCGTACTATTGTTGGTTTTATTCGTATGAGACATT
TGGAAAGTTCAGCGTTGAGGCTGAGTGCCCCAAATTCATTGCTTGGGTGACGAGATG
CTTGCAGAGAGAGACTGTTGCCAAGGCACTTCCTGATGAGAAAAAAGTGTTTGAATT
TGTTTCAGTGTTGAGGGAAAAAATTAGAGTTGAGTAA
>Cs7g27960
(SEQ ID NO: 40)
ATGGCAACAAGCAATTGTGCTGATGCACTTTCACTCATTTTCTCGCCTTCCTTCTCCT
CCAATACAGCATCGAAGCTGCCCCATATCAGAAAATTTTCTCAAACCAATCTTTGCT
TGCATTATCACAACACGCTCTCCTCTGTCCTACGGAACAAGAATGCCATAGCTGCTT
GTGTATCAGCGACAATGGCTTCCAATGTTGTGAAAGAAGTTTTGCCACCAGCTCTTG
ATTCTACTTCAGAACCCCCTCCATTATTTGACGGAACCACAAGGTTGTATATATCGT
ACACGTGTCCATATGCACAGCGTGTGTGGATTACGAGGAACTGTAAGGGATTGCAG
GAGAAGATTAAATTGGTTCCTATTGATTTGCAAAACAGACCTGATTGGTACAAGGAG
AAAGTCTATCCGGCGAACAAGGTGCCGTCATTGGAACATAATAATGAAGTTAAAGG
AGAGAGTCTCAATTTGATGAAATACATTGACAGTCACTTCGAAGGGCCTTCTCTTTT
CCCTGATGATCCTGCCAAGAGAGAATTTTCAGAAGAGTTGTTTTCCTACACTGACAC
ATTCAATAAAACCGTGCGTTCTTCGGTCCAAGGAGATGGAAATGAAGCCAGTACTG
CATTTGATTATTTTGAAACGGCTCTCTCCAAGTTCAATGATGGGCCTTTTCTTCTGGG
CCAGTTCAGCCTAGCAGATATAGCTTATGCTCCATTCATTGAAAGATATCTGCCTTTC
TTACTTGAAGTAAAGAAGTATGACATCACTGCAGGGAGGCCTAAACTGGCTACTTG
GATTGAGGAGATGAACAAGAATGAGGCTTTCAATCAAACCCGCCGTGATCCTAAGG
AGCTTGTTGAAACCTACAAGAAAAGATTTGCGGCTAAGATCTGA
>orange1.1t03622
(SEQ ID NO: 41)
ATGGCTGAAGTGAAGCTTCATGGGGCATGGGCGAGTGCATTTTGTTACAGAGTGATA
TGGGCTCTGAAATTGAAAGGCATAGCATATGATTACATTGAAGAAGATCTCTCTAAC
AAAAGTGCTTTGCTTTTGAAGTATAATCCAGTTCACAAGAAGGTCCCAGTGCTTGTT
CATGACTCAAAATCAGTTTGCGAGTCCATTGTTATCCTTGAATACATTGAGGAGATG
TGGCCACAGAATCCCTTAATGCCAAATGATCCTTATGATAGAGCTCTGGCTCATTTTT
GGATTATATTTGCCTAA
>Cs5g15170
(SEQ ID NO: 42)
ATGGCGGACGAAGTGATTCTGTTGGATTTCTGGCCCAGCATGTTCGGGATGAGGGTC
AGGATTGCTCTGGCGGAGAAAGGCGTCAAGTATGAATACAAGGAAGAGGACTTGAA
GAACAAGAGCCCCCTGCTTCTGCAGATGAACCCGGTTCACAAGAAGATCCCGGTTCT
CATCCACAACGGCAGACCGGTCTGTGAGTCATCCATCATTGTTCAGTACATTGATGA
AGTGTGGAAGGACAAGGCTCCATTGCTTCCCTCTGATCCCTACCAGAGAGCTCAAGC
TAGGTTCTGGGTAGATTTCATTGACAAGAAGATGTACGATGCTTCGACGAAGACATG
GACCACAAAGGGAGAAGAGCAGGAGGCGGCTAAGAAGGAGTTCTTTGAAGCTCTG
AAGACCTTGGAAGGGCAGCTTGGAGACAAGCCTTTCTTCGGAGGAGACAATTTTGG
GTATTTGGATTTGAGTTTGATCCCATTCTATTACACGTATGAGACATTTGGAAAGTCC
AGCGTTGAGGCTGAGTGCCCCAAGTTCAAAGCTTGGGTGAAGAGATGCTTGCAGAG
GGAGACTATTGCCAAGGCACTTCCTGATGAGAAGAAAGTGCTTGAACATGCTTCAG
GGATGAGCAAAAATTTAGTATTGGAGTAG
>Cs8g19380
(SEQ ID NO: 43)
ATGGCTGAAGAAGAAGTGAAGCTCTATGGCACATGGGTAAGCCCTTTTAGTCGCAG
AATCGAGCTTGCACTGAAACTGAAAGGGGTTCCGTTTGAGTACATAGGAGTAGATCT
GTCCAACAAGAGTCCTGAACTTCTGAAATACAATCCAATTCACAAGAAAATCCCAGT
GCTTGTACACAATGGCAAATCAATTGTTGAATCTCTAATCATTCTCGAGTACATCGA
CGACACGTGGAAGAATAATCCTATTTTGCCTCGAGATCCTCATCAAAGAGCCGTGGC
TCGCTTCTGGGCTAAGTTCATCGACGAAAAGCTGTTGGCAACAGGAATGAAGGCCA
GTTTAGCTGAAGGGAAGGAGAAAGAGCTGTTGAATGAAGAAATACTTGAGCAGATG
AAATTGCTGGAGAATGAACTCAATGGAAAAGACTTCTTTGGAGGTGAGGCGATTGG
GCTTGTTGACATTGTTGCAACTGTGGTCGCATTTTGGTTCCCAGTAAGCCATGAAGTT
CTTGGAGTAGAAGTAATCACTCAGGAAAAGTTTCCAGTTTTACTCAAATGGATTGGG
AAGCTTCAAGAGATTGATGTGGTGAGCCAAAGCCGACCTCCAAGAGAGAAGCATGT
TGCTCATGTTAGAGCTCGCATGGAAGGCCTCAATTCAGGTTCAAAGTAA
>orange1.1t03624
(SEQ ID NO: 44)
ATGCCGGTGATAAAATCAGATGTAAAATATTTGTGGCAGGGTGTTGCTGTTTGGAAA
ATGTTTCGTAGCACTGGACAAGAACTACAAAACACAATGAAGGAAGCCTTGGAAAT
GCTACAAACCGTTGAAGAGCATGGCTTGGGAGAGAAGAAATTTTTCAATGGAGACA
ATATTGGCTTAGTGGACCTAGCGTTTGGTTCAATTGTTTATTGGTTGCAAGTCATTGA
AGATGCAGTGGGAGTTAAACTATTTGAATCACACAAATTCCCTCGCTTGCATGCCTG
GCTTGAAAATTTCAAGCAAGTCCCCATAATTGAAGAAAACCTTCCCAATCAGGATGA
AATGTTGGTTGTTTTCAAACGCCGTCGCGAACAGTTGGTGGCTTCTGCTTGA
>Cs5g15190
(SEQ ID NO: 45)
ATGGCGAACGAAGTGGTTCTGTTGGATTTCTGGCCCAGCATGTTTGGGATAAGGGTC
AGGATTGCTCTGGCGGAGAAAGGCGTCAAGTATGAGTACAAGGAAGAGGACTTGAA
GAACAAGAGCCCCCTGCTTCTGCAGATGAACCCCGTTCACAAGAAGATCCCGGTTCT
CATCCACAACGGCAGACCGTTCTGTGAGTCACCCATCATTGTTCAGTACATTGATGA
AGTGTGGAAGGACAAGGCTCCATTGCTTCCCTCTGATCCCTACCAGAGAGCTCAAGC
TAGGTTCTGGGTAGATTTCATTGACAAGAAGATGTACGATTCTTTGAGGAAGACATG
GACCACAAAGGGAGAAGAGCAGGAGGCAGCTAAGAAGGAGTTCTTTGAAGTTCTGA
AGACTTTGGAAGGGCAGCTTGGAGACAAGCCTTTCTTCGGAGGAGACAATTTTGGGT
ATGTGGATTTAAGTTTGATCCCATTCTATTGTTGGTTTTACACGTATGAGACATGTGG
AAAGTTCAACGTCGAGGCTGAGTGCCCCAAGTTCATAGCTTGGGTGAAGAGATGCTT
GCAGAGGGAGACTGTTGCCAAGGCACTTCCTGATGGGAAGAAAGTGTTTGAATTTG
TTTCAGCGATGAGGACAAATTTAGGATTGGAGTAG
>Cs8g19390
(SEQ ID NO: 46)
ATGGCTGAGGAAGTGAAGCTCTATGGCACATGGGTAAGTCCTTTTAGCCGCAGAATC
GAGCTTGCACTGAAACTGAAAGGGGTTCCATTTGAGTACATAGAAGAAGACTTGTC
CAACAAGAGTCCTGAACTTCTTAACTACAACCCAGTTCATAAGAAAATCCCAGTGCT
CGTACACAATGGTAAACCAATAGCTGAGTCCCTAGTCATTCTTGAGTACATTGATGA
GACATGGAAGAATAATCCCATTTTGCCTCAAGATCCTTATCAAAGAGCCATGGTTCG
CTTCTGGGCCAAGTTCATTGATGAGAAGGTCTTGGCCACAGGATGGAAGGCTAATTT
TGGTGAAGGGAAGGAGAGAGAGGTTGCGATTGAAGAATTTGCTGAGCAGATGAAAT
TCTTGGAGAATGAACTCAATGGAAAAGACTTCTTTGGCTATTGGGTTTCTTGA
>orange1.1t03626
(SEQ ID NO: 47)
ATGACAAGGGGAGCCACTGTTTGGAAAATGTTTCGTAGCGTACAAGATCAAGAGAA
TACAATGAAAGAAATCTTGGAAATGCTAAAAACCGTTGAAGAACATGGCTTAGGAG
AAAAGAAATTTTTTCATGGAGACAAGATTGGCTTAGTGGACATAGCATTTGCTTCCT
TTGTTTATTGGTTGCAAATCATTGAAGATGTAGCAGGAGTGAAACTATTTGAATCTC
ACAAATTCACTCGCTTGCACGCATGGTTTGAAAAATTTAAACGAGTCCCAGAAATTG
AAGAGAACCTTCCTAATTGGGGTGAACTATTGGTTTTTTTCAAACGTCGCCGTGAAA
AGTTGGTGGCCTCTTCCTCATTGCATTAG
>Cs5g34430
(SEQ ID NO: 48)
ATGGAACAAGTTAAGCTAATAGGAGCCTGGCCAAGTCCCTATGTTTACAGAGTCATA
TGGACTCTGCAACTTAAAGGCATCAAGTACGAATATGAAGAAGAAAACTTGTCGAA
CAAGAGTGACATGCTTTTGAAGTACAACCCAGTTCATCAAAAAGTCCCTGTGCTTGT
TCATGATGAGAAACCAATCGTTGAGTCCACTGTTATTCTTGAGTACATAGAGGAGGC
ATGGCCTCGGCATCCTTTGCTCCCCAAAGACCCTTATGATAGAGCCGTGGCGCGATT
CTGGATTAATGCTGTGTTTACTTGCTGGAGTGAGACATTCTATATTGGTGTTGGAGA
GGAGCATGAGAAGGCGATAAAAGAAGCAAAGGAGAAATTGAAAATCATTGAAGAA
CAAGGCCTCGGAGACAAGAAATTTTTTGGGGGCAATGAAATTGGGATGGCAGATCT
CGTCTTTGGATGGATAGCTAAATCTTTTGGAGTCGTGGAAGAAGTAGTTGGTGTCAA
AGTTCTTGACGCCGATAGCTTCCCTCGTTTACATGCTTGGATTGGGAGTTTCAGGAGT
CACCCTGTGATTAAAGAGCATCTTCCTGATTGTGATGAGATGTTTGCTTACTACAAG
CAGAAAAGGGACATCACGGAAGAAGTGAAGCTACTGGGAACTTGGCCGAGTTCATT
TTGCTACAGAGTTATATGGGCTCTAAAACTCAAAGGATTGGAATACGAGTATGTGGA
AGTGAATATTCACAACAAGAGTGAGTTGCTTCTCCAGCTTAACCCAGTCCACAAACA
AGTTCCAGTGCTTGTTCATGGTGGAAGGCCAGTTGCAGAATCAATGGTTATTCTTGA
GTACATAGAAGAGGTCTGGCCTCAGAATCGCCTGCTTCCAGATGATCCCTCTGGGAG
AGCTGAGGCTCGGTTCTGGACAAAATTCGGTGAAGACAAGACTCCAACGTTTTTTGC
ATTCTTTCATGCTGTTGGGGAAGAGCAAGAGAAGGCTATAAGAGAAGCGAAAGAAA
TTTTGAAGATCTTGGATGAGAAAGGACTTGGGAAGAAGAAGTTCTTCGGAGGTGAC
GAGATCGCTTTGACTGATATAGCTTTTGGATGGATAGCTGGGTGGGTTGGAGCTATG
GAAGAAGCAGTCGGAGTTAAGCTACTGGACCCAGATAGCTTCCCTCGATTACAGGC
CTGGGTTGAGAACTTTAAGGAAGTTCCAGTGATCAAAGAGAATCTTCCTGATCATAA
TGAATTGTTGGTCTACTTCAAACGCCTGAGGGATATGTTTATTGTATCAGCTACTTCA
TAG
>Cs8g19400
(SEQ ID NO: 49)
ATGGCTGAAGAAGTGAAGCTTTATGGCACATGGTCAAGCCCTTTTAGTCGCAGAATC
GAGCTCGCACTGAAACTGAAAGGCGTTCCCTTTGAGTACATAGAAGAAGACTTGTCC
AACAAGAGTCCTGAACTTCTTAAATACAATCCAGCTCACAAGAAAATTCCTGTGCTT
GTACACAATAGCAAACCAATAGCTGAGTCACAAGTCATTCTTGAATACATCGATGA
GATATGGAATAACAATCCCATTTTGCCTCAGGATCCTTATCAAAGAGCCATGGCTCG
CTTTTGGGGCAAGTTCATTGATGAGAAGGTATTGGCGACGAGAATGAAGGCTAATTT
TGCTGAAGGGAAAGAGAGAGAGCTTGTGACTGAAGAATTCTTCCAGCAGATGGAAT
TCCTGGAGAATGAGCTCAATGGAAAAGATTTCTTTGGAGGTGAAACTATTGGGTTTG
TTGACATAGTTGCAATTGTTGTGGCAATTTGGTTCCCGTTGACACAAGAAGTTCTTG
GAGTAGACACATTCACTGAAGAAAAGTTTCCAGTTTTGTTCAAATGGATTCGGAAGA
TCACAGAGATTGATGTGGTTAACGAATGCCGACCTCCAAGAGAGAAGCATCTTGCCT
ATGTTAAAGCTCGCATTGAAGACCTCAAATCTGCTTCCAAGTGA
>orange1.1t03628
(SEQ ID NO: 50)
ATGCCGGTGATAAAATCAGATGTAAAATATTTGTGGCAGGGTGTTGCTGTTTGGAAA
ATGTTTCGTAGCACTGGACAAGAACTACAAAACACAATGAAGGAAGCCTTGGAAAT
GCTACAAACCGTTGAAGAGCATGGCTTGGGAGAGAAGAAATTTTTCAATGGAGACA
ATATTGGCTTAGTGGACCTAGCGTTTGGTTCAATTGTTTATTGGTTGCAAGTCATTGA
AGATGCAGTGGGAGTTAAACTATTTGAATCACACAAATTCCCTCGCTTGCATGCCTG
GCTTGAAAATTTCAAGCAAGTCCCCATAATTGAAGAAAACCTTCCCAATCAGGATGA
AATGTTGGTTGTTTTCAAACGCCGTCGCGAACAGTTCTTGTTTTGCTTTTGTTTTGGT
ATCTAA
>Cs6g03820
(SEQ ID NO: 51)
ATGGCAACCACAGCGCAAGAGAATCTTCCTCCACCGTTGGATTCGAAGGCCGAGCT
ACCACCTCTGTTCGATGGAACTCCCAGGTTGTACATGTCTTACTCGTGCCCATTTGCT
CAGCGTGCGTGGATCACCAGGAACTACAAGGGATTACAAGACAAGATTAAGTTAGT
TCCACTGAACCTCCAAGATAGGCCCGCTTGGTACAAGGAGAAGGTTTATCCTGTTAA
CAAGGTTCCAGCACTGGAGCACAATGGCAAAGTTATTGGAGAATCTCTTGATTTGAT
TAAATATGTTGACAGCAATTTTGAAGGGCCTTCACTTTTACCTGATGATCCTGAGAA
ACGAAAGTTTGCTGATGAACTGTTTTCCCACATCGATACCTTCACCAACGATGTGTA
TACTTCATTCAAAGGAGACCCAGCAAAACAAGCTGGTCCTGCTTTTGATTACTTAGA
GAAGGCCCTTGATAAATTTGATGATGGCCCGTTCTTCCTTGGACAGTTCAGTCAGGT
GGACATAGCCTATATTCCATTTGTTGAGAGATTCCAGATCTTCTTATCAGAAGAATTT
AAGTATGACATCACTGCAGGCAGGCCTAAACTGGCAGCATGGATTGAGGAGTTGAA
CAAGCTCGATGCTTACAAGTCAACAAAAGCAGATCCAAAAGAACTCGTTGAATTTT
ACAGGAGCCGCTTCGTGCCAGTTTAG
>Cs9g10400
(SEQ ID NO: 52)
ATGGCAACAAAAGTGAAGGTTTTGGGGTTTTGGTGTAGTCCTTTTGTGTTTAGGGTA
GTATGGGCATTGAAGCTAAAGGGTGTTGAATTTGAATACATTGAAGAAGACATCTTT
AACAAAAGCACCAGGCTTCTTGAGCTGAACCCAGTTCATAAAAAGGTTCCTGTGCTT
GTTCATGATGACAAAGTGATAGTTGAGTCGTTTGTTATTCTGGAGTACATCGATGAG
ACATGGCCACAAGATCCGTTGCTGCCTCAAGATCCTTATGAAAGAGCCATGGCTCGA
TTTTGGGCTAAATTTGCAGATGAAAAGCTGATGCAAACTGCATGGAAGTCAACGTG
GACCAAAGGAGAGGAAAAAGAGGATGCTCTGAAAGCAACAAAGGAAGTTTTGGAG
AAGATAGAGGGAGAACTCAAAGGAAAAAGCTACTTTGGAGGAGAGAACAAATATG
CGTATTTGGAAGTTGCAATTGGATGGATCAATTACCTTATTCCAATTTGGGAAGAAA
TTGCATCCATTCAATTTCTTGACCCGAAAAGATTTCCAGCCATTACTGCATGGAAAA
GCAAGTTTCTGGATAACCCTTTGATCAAAGAAACCCTGCCACCGAGGGAGAAATTG
CTTTTCTATTTGCACGAGCGCATCAAACAACGGGAGATTGACGCTACAATCCGTGAA
CTGGCGGAGACCAAACTTGAAAATTAA
>orange1.1t03629
(SEQ ID NO: 53)
ATGGCTGAAGTGAAGCTTCATGGGGCAGTACTGCCGAGTCCATTTGTTTACAGGGTG
ATTTGGGCTCTGAAATTGAAAGGCGTGCCATATGAATTTGTTGCAGAAGATCTCTCA
AACAAAAGTGCTTCGCTTTTGAAGTACAATCCAGTTCACAAGAAGATCCCAGTGCTT
GTCCATGGCGGAAAACCAGTTTGTGAGTCCATGGTTATTCTCGAATACATCGAAGAG
ACGTGGCCACAGAATCCCTTGATGCCAAACAATCCTTATGATAGAGCTCTTGCTCGA
TTCTGGATTAAATTCGCCGAAGACAAGGGTGTTGCTGTTTGGAAAATGTTTCGAAGC
ACTGGACAAGAACTACAAAACACAATGAAGGAAGCCTTGGAAATGCTACAAACCAT
TGAAGAGCATGGCTTGGGAGAGAAGAAATTTTTCAATGGAGACAATATTGGCTTAG
TGGACCTAGCGTTTGGTTCAATTGTTTATTGGTTGCAAGTCATTGAAGATATAGTGG
GAGCTAAACTATTTGAATCACACAAATTCCCTCGCTTGCATGCCTGGATTGAAACTT
TCAAGCAAGTCACTTTGATTGAAGAAAATCTTCCTAATCAGGATGAACTATTGGTTG
CTCTCAAACTTCGTCGTGAACAGTTGATGGCTTCTGCTTGA
>Cs6g03830
(SEQ ID NO: 54)
ATGGCTTCTGTTGTGTACGAGAATCTTCCTCCAGTATTGGATTCAAAATCCGAGCAA
CCTCCGCCACCTTTATTCGATGGAACTACCAGGCTCTATATGGCTTACACGTGCCCA
TTTGCTCAGCGTGTGTGGATCACCAGGAACTATAAGGGATTACAAGATGAGATTAA
GTTGGTTGCCTTCGATCTTCAAGATAAGCCTGCTTGGTACAAGGAGAAAGTTTACCC
TTCTAACAAGGTGCCTTCACTTGAACATAATGGCAAAATTATTGGGGAGAGTCTTGA
TTTGATTAACTATGTTGACAGCAATTTTGAAGGGCCTTCGCTTTTACCTGATGATCCT
GAGAAAAGAAAGTTTGCTGAAGAATTGTTTTCCTACAGTGATACATTCATCAAAGAT
GTATCTACTTCATTCAAAGGAGACACGGCAAAAGAAGCTGGTCCTGCTTTTGATTTC
TTGGAGAAGGCTCTTGATAAGTTTGATGACGGGCCATTCTTCCTTGGCCAGTTCAGT
TTGGTGGACATAGCCTATATTCCATTTGTTGACGGTTACCACATGATCTTATCAGAA
GGATTTAAGTATGATGCCACAGTAGGCAGGCCTAAACTTGCAGCATGGATTGAGAT
GTGA
>Cs9g10410
(SEQ ID NO: 55)
ATGGCAACAAAAGTGAAGGTTTTGGGGGTTTGGTGTAGTCCTTTTGTGTTTAGGGTA
GTATGGGCATTGAAGCTAAAGGGTGTTGAATTTGAATACATTGAAGAAGACATCTTT
AACAAAAGCACCAGGCTTCTTGAGCTGAACCCAGTTCATAAAAAGGTTCCTGTGCTT
GTTCATGATGACAAAGTGATAGTTGAGTCGTTTGTTATTCTGGAGTACATCGATGAG
ACATGGCCACAAGATCCGTTGCTGCCTCAAGATCCTTATGAAAGAGCCATGGCTCGA
TTTTGGGCTAAATTTGCAGATGAAAAGTCAACGTGGACCAAAGGAGAGGAAAAAGA
GGATGCTCTGAAAGCAACAATGGAAGCTTTGGAGAAGATAGAGGGAGAACTCAAA
GGAAAAAGCTACTTTGGAGGAGAGAACAAATATGCGTATTTGGAAGTTGCAATTGG
ATGGATCAATTACCTTATACCAATTTGGGAAGAAATTGCATCCATTCAATTTCTTGA
CCCGAAAAGATTTCCAGCCATTACTGCATGGAAAAGCAAGTTTCTGGATAACCCTTT
GATCAAAGAAACCCTGCCACCAAGGGAGAAATTGCTTATCTATTTTCACGAGCGCGT
CAAACATTGGGAGATTGACGCTACAATCCGTGAACTGGCGGAGACCAAACTTGAAA
ATTAA
>orange1.1t03630
(SEQ ID NO: 56)
ATGACTGAAGTGAAGCTTCATGGGGTATTGCTGAGTCCATATGTTTACAGAGTGATT
TGGGCTCTGAAATTGAAAGGCGTGCCATACGAATTTGTTGCAGAAGATCTCTCAAAC
AAAAGTGATTTGCTTTTGAAGTATAATCCAGTTTATAAGAAGATCCCTGTGCTTGTC
CATGGCGAAAAACCAGTTTCTGAGTCCATGGTTATTCTCGAGTACATTGAAGAGATG
TGGCCACAGAATCCCTCCTTGATGCCAAACGATCCTTATGATAGAGCTCTTGCTCGA
TTTTGGATTAAATTCATCGAAGACAAGGTTCGCACCACAACTCTAGCAATCCCGTTT
ATAAATATATTCTTCTCAAATTTATTTATTTATTTGAGGTTTCGATTAGACCATAAGA
ATAAAGAACTTCACCCACTAAAACCCCAACTCTTGTCCTTTGTTACTCGAACTCGGG
TCATAGTACGTATAGCGGATTCACAGTCAATCAAGCCTAGTCGCTCAATAGCCTTTT
TCATATTTATTTGTAAAAAACTCTTTAAACTTTCAACTCCTTGTTTCTTCACTACTTTG
TTTCTAGAAATTTTTATTAACCAGTAG
>Cs6g03850
(SEQ ID NO: 57)
ATGGCGACTGTTGTTCATGAGAATCTTCCTCCAGTGTTGGATTCTAAATCCGAGCAA
CCACCATCTGTTTTCGATGGAACTACCAGGCTGTATATGGCTTACACGTGCCCATTTG
CTCAGCGTGTGTGGATCACCAGGAACTATAAGGGATTACAAGATGAGATTAAGTTA
GTTGCCATAAGCCTTGAAGATAAGCCTGTTTGGTACAAGGAGAAAGTTCACCCTGCT
AACAAGGTGCCTGCGCTTGAACACAACGGCAAAATTGTTGGTGAGAGTCTTGATTTG
ATTAAGTATGTAGACAGCAATTTTGAAGGGCCTTCACTATTACCTGATGATCCTGAG
AAAAGAAAGTTTGCTGAAGAATTGTTTTCCTACTCTGATACATTCAACATCACTGTG
TATGGTTCATTCAAAGGAGACCCGGCAAAGGAAGCTGGTCCTTGTTTTGATTACTTG
GAGAAGGCTCTTCATAAGTTTGATGACGGTCCATTCCTCCTTGGCCAGCTCAGTTTG
GTGGACATAGCCTATATTCCATTTGTTGAAAGAGCCCAGATCTTCTTATCAGAAGTA
ATCAAGTATGACATCACAGCAGGCAGGCCTAAATTGGCAGCATGGATTGAGGAGTT
GAACCAGGTCGATGCTTATAAGCAGACAAAATTAAAAGATCCCAAAGGGCTCGTTG
AACTTTACAAGATCCGCTACATGTGA
>Cs9g10420
(SEQ ID NO: 58)
ATGGCAACTAAAGTAAAGCTTTTAGGGTTTTGGTCTAGTCCTCCTGTGTTTAGAGTA
GTATGGGCACTGAAGCTAAAGGGTGTTGAATTTGAAAACATTGAAGAGGATATCTTT
AACAAAAGCACCAGGCTTCTTGAACTGAACCCAGTTCATAAAAAAGTTCCTGTGCTT
GTTCACGATGACAAAGTGATAGTTGACTCGTTTGTTATTCTGGAGTACATCGATGTG
ACATGGCAACATGATCCTTTGTTACCTCAAGATCCTTATGAAACGGCCATGGCTCGA
TTTTGGGCTAAATTTGCATATGAAAAGCTTTTGGAATCAGCATATAAGGCAATGTGG
AGCAAAGGAGAGGAGAAACAAAAGACTATGAAAGAAGCAATAGAATCCTTGGAAA
AGATAGAAGAACTCAGAGGAAAGAACTTCATCGGAGGAAATAGCATTGGTTATTTG
GACCTTGCAATTGGATGGATCAGTTACTGGCTTCCTGTTTGGGAAGAAGCTGATGGA
TCCATGCGAATTCTTGATTCTCAGAAATTCCCAAACCATTGCCGATGA
>orange1.1t03632
(SEQ ID NO: 59)
ATGGCTGAAGTGAAGCTTCATGGGGCACTGCTGAGTCCATTTGTTTACAGGGTGATT
TGGGCTCTGAAATTGAAAGGCGTGCCGTATGAATTTGTTGCAGAAGATCTCTCAAAC
AAAAGTGCTTTGCTTTTGAAGCACAATCCGGTTCACAAGAAGATCCCAGTGCTTGTC
CATGGCGGAAAACCAGTTTGTGAGTCCATGGTTATTCTCGAATACATCGAAGAGACG
TGGCCACAGAATCCCTTGATGCCAAATAATCCTTATGATAGAGCTCTTGCTCGATTC
TGGATTAAATTCGCCGAAGACAAGGGTGTTGCTGTTTGGAAAATGTTTCGTAGCACT
GGACAAGAACTACAAAACACAATGAAGGAAGCCTTGGAAATGCTACAAACCGTTGA
AGAGCATGGCTTGGGAGAGAAGAAATTTTTCAATGGAGACAATATTGGCTTAGTGG
ACCTAGCGTTTGGTTCAATTGTTTATTGGTTGCAAGTCATTGAAGATGCAGTGGGAG
CTAAACTATTTGAATCACACAAATTCCCTCACTTGCATGCCTGGATTGAAACTTTCA
AGCAAGTCCCTGTGATTGAAGAAAATCTTCCTAATCAGGATGAACTATTGGTTGCTC
TCAAACTTCGTCGTGAACAGTTGATGGCTTCTGCTTGA
>Cs6g07240
(SEQ ID NO: 60)
ATGGCTCAAGTGCAGTTGTTTGGATTATGGGGAAGCCCTTTCAGTTGCAGAGTAGAG
ATAGCTTTGAAACTGAAAGGAGTCGAATACGAGTACATAGAAGAAGATCTTTCTAA
CAAGAGCCCTTTGCTTCTACAATACAACCCCATTCACAAGAAAGTTCCTGTTCTTGTC
CACAAAAGAAAGCCAATTGCTGAGTCACTTGTCATACTCGAATACATTGATGAGACC
TGGAACGAACGCAATCCCATCTTGCCTCACGACCCCTATGAACGAGCCACGGCGCG
ATTCTGGGCGAAGTTCATCGATGAAAAGTGCATCTTTGCTCTGTGGAAAGCCTGCTG
GAGTGAAGAGAAAGAAAGGGAGGAGGCTGCGAGAGAAGCCTGCGAGCACTTACAA
ACGCTAGAAGATGTGCTCAAAGATAAGAGGTTCTTTGGTGGAGACATGATCGGGCT
AGTTGATATTGTTGCGAACTTCATGGGCTTTTGGCTCGGAATCATTGAAGAAGCCAC
GGGAGTTAAGTTATTGACAGAAGAGAAATATCCGAAATTATACAAATGGAGTGAAG
AGTTTGTCAATTGCAGCGCTATCAAGGAAAATCTGCCTCCAAGAGACAAGCTTCTTG
CCTATATGCGTGCTCGTTATGGAGTGTCCATAACTTACTAG
>Cs9g10430
(SEQ ID NO: 61)
ATGACAACAAAAGTGAAGCTTTTAGGGTTTTGGTGTAGTCCTTTTGCGTTTAGGGTG
GTATGGGCACTGAAGCTAAAGGGGGTTGAATTTGAATACATTGAAGAAGATATCTTT
AACAAAAGCACCGAGCTTCTTGAATTGAACCCGGTTCATAAAAAGGTTCCTGTGCTG
GTTCATGACAAGAGAGTGATAGTTGAGTCGTTTGTTATTCTGGAGTACATCGATGAG
ACGTGGCCACAAGATCCGTTGCTGCCTCAAGATCCTTACGAAAAAGCCATGGCTCG
ATTTTGGGCTAAATTTGGAGATGAAAAGCTTTTGGAATCAGCATATAAGGCAATGTG
GAGCAAAGGAGAGGAGAAACAAAAGGCTATGAAAGAAGCAGTAGAATCCTTGGAA
AAGATAGAAGAAGTACTCGGAGGAAAGAACTTCATGGGAGGAAATAGCATTGGTTA
TTTGGACCTTGCAATTGGATGGATCGGTTACTGGATTCCTGTTTGGGAAGAAGCTGG
TGGATCCATGCGAATTCTTGACTCTCAGAAATTCCCAGCCATTGCTGAATGGAGCGC
CAAGTTTCTGAAACACCCTGTCATCAAAGAAAACTTGCCACCCAGGGACAGGACGC
TTGCCTATTGTCACAAGCGAGCAGAAGAAATATATGCTAACTTTTGTGCATCGGAGA
ATTGA
>orange1.1t04722
(SEQ ID NO: 62)
ATGTGTGAATCCACTATTATAGTTAACTACATTGACGAGACTTGGCCTTCGCCACCA
CTTCTCCCATCCTGTGCTTATGAGAGAGCTAAGGCAAGGTTCTGGGCAGATTTTATT
GACAAGAAGGTATTCGATGCAGTATGCAACACATGGAAAAGCAAGGGCGAGGTCCC
AGAGACGGCGAAGAATGAATTTATAGAGATTTTGAAGCAACTTGAGGGAGCTTTAG
GGGAGAAGGATTTCTTTGGCGGGGATAGCTTTGGATTCGTTGATGTCATTGCCATTC
CACTGACTTGTTGGTTTTACGCGGTTGAGAAATTTGGGGGATTTAATGTTGAGAATG
AGTGCCCCAAATTCTCAGCTTGGATGAACAAGTGCATGCAAAGAGAAACTGTTGCC
AGGATCCTTCCAGACCCAGAAAAGGTTACGAATTTGTTATCACGTTGA
>Cs6g07260
(SEQ ID NO: 63)
ATGGCTGAAGTGCAGTTGTTTGGTCTATGGGGAAGCTTCTTCAGTCACAGAATAGAG
ATTGCTTTGAAACTGAAAGGTGTTGAATACGAATTCATAGAAGAAGATCTTTCTAAC
AAGAGCCCTTTGCTTCTCCAGTACAACTCAATTCACAAAAAAGTTCCAGTTCTCGTA
CACAAAGGGAACCCAATTGCCGAGTCACGTGTCATACTTGAATATATCGATGACACC
TGGAAAGGCCATCCCATTTTGCCAGAAAATCCCCATGAACGAGCAAATGCACGTTTC
TGGGCCCAGTTCATCGATGAAAAGTGCAGGGTTGCCCTGAGGAATGCTTACGGGTG
CCAAGAGAAAGAACGAGAGGAGGAGGCCACAAGAGAGGCGTGTGAGCTCCTGAAA
ACACTAGAAAATGAGCTCAAAGATAGGAGGTTCTTTGGTGGAGACAGGATTGGGCT
GGTTGATATTGTTGCAAATTTCATAGGCTTTTGGCTCGGGGCCATTCAAGAAGCACT
TGAGGTCAAGTTGTTTACGGAAGAGAGGTTTCCGAAATTATACAGATGGAGTGAAG
AGTTTGTTAATTGCAGTATCATCAGGGAAAGTTTGCCCCCAAGAGACAAACTTATTT
CCTTTATGCGTCGTCGTTATGGACTCTCTAGTTAG
>Cs9g10440
(SEQ ID NO: 64)
ATGGCTACAGAAGTCAAGCTCTTAGGGATTTGGCCAAGCCCGTTTGTGTTTAGGGTA
AAAGTGGCACTGCAGCTGAAGGGTGTTGATTACGAATACTTTGAAGAAGATCCCTTC
AACAAAAGTCCCAGGCTTCTAGAATCGAACCCAATTCATAAGAAGGTTCCTGTGCTT
ATTCATGGCAAGAAACCGATCAATGAGTCATTGACTATTCTTGAGTACATCGAGGAG
ACATGGCAGAATAATCCTTTGTTGCCTCAAGATCCTTACGAAAGAGCCACGGTTCGT
TTCTGGGCTAAATTTGTAGATGATTTGTTTTGGAATAAAGCATTTGCGGCATTCATTG
CGAAAGGAGAGGCAAAAGAGAAGATTGAAAAAGAAGTAATAGAAGGATTGGAAAA
GATAGATGGAGCAATCAAAGAGAAGAGCAGCCTGTTTAATAGTGCTGCCGGAGGAG
AGAGCATTGGTTTTTTGGACATTGTATTTGGAGTCATCGGTTACTGGCTGCCTATTTA
TGAAGAAGCCGGTTCAATGCAAACACACACTCAGAAATTTCCAGCCATTGCTGAAT
GGACCACCAAGTTTGTCAATCACCCTGTCATCAAAGAAAACTTACCATCCACTGAGA
GCTTGCTTCCGTTTTTCCGCCAAGTCCATGAGTTTGTATCATCAGCATCCAACAACTA
A
>orange1.1t04724
(SEQ ID NO: 65)
ATGCCACATCCTTTAAGGAAGTGCAACGAACAAACGTTGATTGCGACACAAACTTTG
GGAATTTCATCTTTTCTCCTTCACAAATGCCAATTAACCTTGATACTCAAGGTAATCG
ATGCAGTATGCAACATATGGAAAAGCAAGGGCAAGGTCCCAGGGACGGCGAAGAA
TGAATTTATAGAGATATTGAAGCAACTTGTGGGAGCTTTAGGGGAGAAGGATTTCTT
TGGCGGGGATAGCTTTGGATTCGTTGATGTCATTGCCATTCCACTGACTTGTTGGTTT
TACGCAGTTGAGAAATTTGGGGGATTTAAGGTTGAGAATGAGTGCATGCAAAGAGA
AACTGTTGCGAGGATCCTTCCAGACCCAGAAAAGGTCTGCGAATTTGTTATCATGTT
GAGGAACATGTTTGGAATTGAACAGTGA
>Cs6g17520
(SEQ ID NO: 66)
ATGTCGACGCTTAGAATTCAACCAAGCACAACAAGTGCAATATCGTCAACTATAAA
GAACCTGCACCTTGGCTTCTCCTTCCGCCTCCCCTGCAAAAACTACGGCGTTTCGAA
GCGACCTCGGAACCTCTCCGTGATGGCCGTGGCTACGGATCCTCTTGAAGTCTGCGT
CAAAGCTTCGTCCGCTTCTCCCAACAGACTCGGCGACTGTCCATTTTGCCAAAGGGT
GTTGTTGACTATCGAGGAAAAGCATCTCCCATATGACATGAAGTTGGTAGATTTGGG
AAACAAGCCAGAATGGTTCTTAAAAATTAGCCCCGAAGGTAAAGTTCCTGTTATAA
AACTGGATGAGAAGTGGCTTCCAGATTCAGATGTTATCACACAATCTCTGGAAGAA
AAATACCCAGATCCGCCACTACGAACCCCGCCTGAGAAGGCTTCAGTCGGATCAAA
GATCTTCTCTATGTTTATTGGTTTTCTGAAAAGCAAAGACCCCAGTGATGGATCTGA
GCAGGCATTGCTGAATGAGCTAAATTCATTCAATGATTACATCAAGGAAAATGGCCC
TTTTATCATTGGGGGAAAGGTTTCTGCTGCAGATTTGTCCCTGGGACCCAAGTTCTAT
CATCTAGAAATTGCTCTGGGGCATTATAAAAATTGGTCGGTTCCAGATTCACTTCCA
CACGTGAAGTCTTACATGAAGACAATTTTCTCAATGGATTCATTTATCAAAACAAGA
GCACTCCCAGATGATGTGATTGTTGGTTGGCGGCCAAAGGTCTTTGGTTAG
>Cs9g10450
(SEQ ID NO: 67)
ATGGCCAAAGAAGTGACGCTTTTGGGGTTTTGGGCAAGTCCATTTGTGTTCAGGGTA
AAAGTCGCTCTGCAGCAGAAGGGTGTTGTTGATTATGAATACATTGAAGAAGACAT
CTTTAACAAGAGCCCCAGGCTCTTGGAACTGAACCCAGTTTATAAGAAGGTTCCTGT
GTTCATCCATGGCGAGAAAGTGGTCAATGAATCGTTGATTATTCTTGAGTACATCGA
TGAGACATGGGAGCAAAATCCTTTGTTGCCTCGAGACCCTTACCAAAGAGCCATGGC
TCGTTTCTGGGCTAAATATTCAGAAGAACTGATGGCGAAAGCATTTATGGCTCTGGT
TTTGAAAGGAGAGGCAAAGGAGAAAAATGCGAAAGAATTTGCAGAAGGATTGGAG
AAGATAGAAGGGGAATTCAAGGGAAAGAGCGGCTTATTGTTTGCTGAAGGAGAGAG
CAATATTGGGTATTTGGAACTTGCATTTGGTTGGATTCTTTACTGGCTGCCTGTTTGG
GAAGAAGCTGGATCCTTTCAAGTTCTTGACCCTCAGAAATTTCCAGTCATCACTGAA
TGGAGCACCAAGTTTGTGAATCACCCTCTCATCAAAGAAAACTTGCCGGCCAGGGA
CCAGATGCTCGTCTATTTCCGCAAAGTCAGTGAAGAGTTCTTTACCTTATTCCGAGC
ATAG
>orange1.1t04916
(SEQ ID NO: 68)
ATGTCCAAGGGAGAGGTTGTTCTTTTGGATTGTTGGGCTAGCCCATTTTGCTTGAGA
GCAAAGATTGCATTGGCTGAGAAAGGAGTTGAATATGAAGCCAGAGCTGAGAACTT
GTTTGGGGGCAAGAGTGACCTGTTGCTCAAATCAAACCCCATCTACAAGAAAGTCCC
AGTTTTGCGGCACGATGGGAAACCATTGTGTGAATCCACTATTATAGTTAACTACAT
TGACGAGACTTGGCCTTCGCCATCACTTCTCCCATCCTGTGCTTATGAGAGAGCTAA
GGCAAGGTTCTGGGCAGATTTTATTGACAAGAAGGTATTTGATGCAGTATGTAACAT
ATGGAAAAGCAAGGGCGAGGTGCCGGAGACAGCGAAGAAAGAATTTATAGAGATT
TTGAAGCAACTAGAGAGAGCTTTAGGGGAGAAGGCTTTCTTTGGCGGGGATAGCTTT
GGATTCGTTGATGTCATTGCCATTCCACTGACTTGTTGGTTTTACGCGGTTGAGAAAT
TTGGGGGATTTAAGGTTGAGAATGAGTGCCCCAAATTCTCAGCATGGATGAACAAG
TGCATGCAAAGAGAAACTGTTGCCAGGATCCTTCCAGACCCAGAAAAGGTCTACGA
ATTTGTTATCATGTTGAGGAACATGTTTGGAATTGAATAG
>Cs7g04580
(SEQ ID NO: 69)
ATGGCAAAAAGCGACGTGAAGGTTTTGGGTTTTTGGCCAAGCCCATTTCTGATGAGG
GCAAGAATCGCACTTAAGATCAAGTCTGTGGAGTATGAGTTTCTGGAGGAGAATTTG
GGATCCAAAAGCGAGCTTCTGCTTAAATCAAACCCTGTTCACAAGAAAATCCCCGTT
CTGATTCACAATGATAAGCCTGTTTGCGAATCTCTCATCATTGTCGAGTACGTGGAT
GAGGCTTGGTCCTCTTCAGGTCCCTCTATTCTTCCTTCTGATCCTTATGACCGTGCCG
TTGCTCGTTTCTGGGCCGCTTACGTTGATGAGAAGTGGTTCTCTGCCTTGAGAGACAT
TGGTAGTGCAAATGGAGCGGAAGCTAAGAAAGCTGCAATTGAGCAATTGATTGAAG
TCTTGGTTTTGCTGGAGGATGCTTTTGTTAAGTGTAGCAAAGGGAAGGCTTTCTTTGG
TGGAAACCAAATTGGGTTTCTTGACATTGCATTTGGTAGCTACTTGGGATGGCTGAG
AGTGACAGAGAAAATGAATGAGGTGAAGCTCCTTGATGAAGTCAAAACTCCTGGAC
TGTTCAAATGGGCTGAGAGATTCTGTGCTGACGCTGCTGTCAAAGATGTTATGCCTG
AGACCGATAAGCTTGCCGAACTCAAAGCTTCTGCTCCTCCTTCCAGCTGA
>orange1.1t03452
(SEQ ID NO: 70)
ATGAACAAGGCGGTGATAAAATCAGATGTAAAATATTTGTGGCAGGGTGTTGCTGTT
TGGAAAATGTTTCGTAGCACTGGACAAGAACTACAAAACACAATGAAGGAAGCCTT
GGAAATGCTACAAACCGTTGAAGAGCATGGCTTGGGAGAGAAGAAATTTTTCAATG
GAGACAATATTGGCTTAGTGGACCTAGCGTTTGGTTCAATTGTTTATTGGTTGCAAG
TCATTGAAGATATAGTGGGAGCTAAACTATTTGAATCACACAAATTCCCTCACTTGC
ATGCCTGGATTGAAACTTTCAAGCAAGTCACTTTGATTGAAGAAAATCTTCCTAATC
AGGATGAACTATTGGTTGCTCTCAAACTTCGTCGTGAACAGTTGATGGCTTCTGCTT
GA
>orange1.1t05889
(SEQ ID NO: 71)
ATGTACGTATGCAATAACTCATCAGCTAAGCAAAACAACCACAAACATATTCAAATT
CGATCTGAGACACATGGCTGAAGTGAAGCTCCACGGGGGATGGGCGAGTCCATTTA
GCAACAAGGTGGTATGGGCTCTGAAATTGAAAGGCATCCCGTATGAATACATTGAA
GAAGATCTCTACAACAAAAGTGCTTCGCTTTTGAAGTATAACCCAGTTCACAAGATG
ATACCAGTGCTCGTTCATGGCGAAAAACCAGTTTGCGAGTCCATGGTTATTCTCGAA
TACATTGAAGAGATGTGGCCACACAATCCCTCCTTGACGCCAAGTGATCCTTATGAT
AGAGCTAAAGCTCGTTTTTGGATTAAATTTGCCGAAGACAAGGGAGTAGCTGTTTGG
AAAATGTTTTTCAGCACTGGACAAGACCTGGAGAATGCAATGAAGGCAGCCTTGGA
AATGCTTCAAACCGTTGAAAAACATGGCCTAGGAGAGAAGAAATTTTTCAATGGAG
ACAATATTGGCTTAGCGGACCTAGCATTTGGAGCCATTGCTTATTGGTTGCAGGTCC
TTGAAGATGTAATGGGAGTGAAACTATTTGAATCTCACAAATTCCCAAGCTTGCATG
AGTGGTTTGAAAATTTCAGGAAAGTACCTGTAATTGAAGAAAATCTTCCAAATCAGC
ATGAGATGGTGATCGCTTTCAAACATCGTCGGGAAACATTGGTGACATCTGCGTGA
>Cs7g04600
(SEQ ID NO: 72)
ATGGCAAAAAGCGACGTGAAGGTTTTGGGTGCGTGGCCAAGCCCATTTGTGATGAG
GGCAAGAATCGCACTTAAGATCAAGTCTGTGGAGTATGAGTTTCTGGAGGAGAATTT
GGGATCCAAAAGCGAGCTTCTGCTTAAATCAAACCCTGTTCACAAGAAAATCCCAGT
TCTGATTCACAATGATAAGCCTGTTTGCGAATCTCTCATCATTGTCGAGTACGTGGAT
GAGGCTTGGTCCTCTTCAGGTCCCTCTATTCTTCCTTCTGATCCTTATGACCGTGCCG
TTGCTCGTTTCTGGGCCGCTTATGTTGATGAGAAGTTGTTCCCTGCCTTGAAAAGCAT
TGGTAGTGCAGATGGAGCGGAAGCTAAGGAAGCTGCGATTGAGCAATTGATTGAAG
GCTTGGTTTTGCTGGAGGATGCTTTTGTTAAGTGTAGCAAAGGGAAGCCTTTCTTTG
GTGGAAGCCAAATTGGGTTTCTTGACATTGCATTTGGTTGCTACTTGGGATGGGTGA
GAGTGACAGAGAAAATGATTGAGGTGAAGCTCCTTGATGAAGTCAAAACTCCTGGA
CTGTTCAAATGGGCTGAGAGATTCTGTGCTGATGCCGCTGTCAAAGATGTTATGCCT
GAGACTGATAAGCTTGCAGAGTTTGCTAAGGTTCTTGCCAAACTCAGAGCTTCTGGC
AAATGGAATTAA
>orange1.1t03455
(SEQ ID NO: 73)
ATGGCTGAAGTGAAGCTTCATGGGGGATTGCTGAGTCCATTTAATTACAGGGTGGTA
TGCGCTCTGAAATTGAAAGGCATACCTTTTGAATTGATTAAAGAAGATCTCTCAAAC
AAAGGTGCATTGCTTTTGAAGTATAATCCAGTTCACAAGAAGATCCCAGTGCTCGTT
CATGGCGGCAAGCCAGTTTGCGAGTCCATGGTTATTCTCGAATACATTGAAGAGACG
TGGCCACAGAATCCATTGATGCCAAGTGATCCTTATGATAGAGCTCAAGCTCGTTTT
TGGATTAACTTTGCCGAAAACAAGGGAGCTGCTGTTTGGAAATTGTTTCGTAGCATT
GAAGATCAAGAGAATACGATGAAAGAAATCTTGGAAATGCTGCAAATTGTTGAAGA
ACATGGCTTAGGAGAAAAGAAATTTTTCCACGGAGACAAGATTGGCTTAGTTGACA
TAGCATTTGGTTCTATTGTTCATTGGTTGCAAATCATTGAAGATATAGTGGGAGTGA
AACTCTTTGAATCTCACAAATTCCCAGGCTTGCACGCGTGGTTAAAAAATTACAAGC
AAGTCCCTGTAGTTGAAGAAAACCTCCCTAGTAGGGATGAACTGTTGGTCTTTTTCA
AAGGTCGCTATGAAAAGTTGCAGGCTTCTGCCAGAATGCAGTAG
>orange1.1t05890
(SEQ ID NO: 74)
AGTGATTTGGGCTCTGAAATTGAAAGGCGTGCCATACGAATTTGTTGCAGAAGATCT
CTCAAACAAAAGTGATTTGCTTTTGAAGTATAATCCAGTTTATAAGAAGATCCCTGT
GCTTGTCCATGGCGAAAAACCAGTTTCTGAGTCCATGGTTATTCTCGAGTACATTGA
AGAGATGTGGCCACAGAATCCCTCCTTGATGCCAAACGATCCTTATGATAGAGCTCT
TGCTCGATTTTGGATTAAATTCATCGAAGACAAGATTGTTGCTATTTTGGGATTGTTT
CGTAGCATTACTGGACAAGAACTAGAGAATGCAACGAAGGAACTCTTGGAAATCCT
ACAAACCCTTGAAGAACATGGGCTGGGAGAGAAATTTTTTTTCAATGGAGACAATA
TTGGCTTAGTTGACATAGCATTTGGTTCAATGCTTTTTTCGATGCAAGTCATTGGAGA
CGTAGTGGGAGTTAAACCATTTGAATCACACAAATTTCCTAGCTTGCATGCCTGGTT
TGAAAATTTCAAACAAATCCCTGTAATTGAGGAAAACTTTCCTAATCGAGATGAATA
TTTTGTTTATATCAAACGTCGCCGTGAAGAGCTGTTGGCCTCTGCTTGA
>Cs7g14120
(SEQ ID NO: 75)
ATGTCCAAGGGAGAGGTTGTTCTTTTGGACTGTTGGGCTAGCCCATTTTGCTTAAGA
GCAAAGATTGCATTGGCTGAGAAAGGAGTTGAACATGAAGCCAGAGCTGAGAACTT
GTTTGGGGGCAAGAGTGACTTGTTGCTCAAATCAAACCCCATCTATAAGAAAGTCCC
AGTTTTGCTACACGATGGGAAACCATTGTGTGAATCCACTATCATAGTTAACTACAT
TGACGAGACTTGGCCTTCGCCACCACTTCTCCCATCCTGTGCTTCTGAGAGAGCTAA
GGCAAGGTTCTGGTCAGATTTTATTGACAAGAAGGTATTCGATGCAGTCTGCAACAT
ATGGAAAAGCAAGGGCGAGGTCCCCGAGACGGCGAAGAATGAATTTATAGAGATTT
TGAAGCAACTTGAGGGAGCTTTGGGGGAGAAGGATCTCTTTGGCGGGGATAGCTTT
GGATTCGTTGATGTCATTGCCATTTCACTGACTTCTTGGTTTTACGCGGTTGAGAAAT
TTGGGGGATTTAAGGTTGAGAATGAGTGCCCCAAATTCTCAGCTTGGATGCACAAGT
GCATGCAAAGAGAAACCGTTGCCAGGATCCTTCCAGACCCAGAAAAGGTCTACGAA
ACTTTATTATGTGCACTCATCTTGAAATCACGGCATGTGCATCTCAGCAGCAAAGAG
TTCTCGGCGGCTACCGGAAGAGGAAGAAATTTCCATGTAAACCAACCTTACAAAGTT
CCAAGGATGCATATGAAAACCTTTTAA
>orange1.1t03456
(SEQ ID NO: 76)
ATGGTTATTCTTGAATATATTGAAGAGATGTGGCCACAGAGCCCCTTGATGCCAAAC
GATCCTTATGATAGAGTTCTTGCTCGTTTTTGGATTAAATTTGGAGAAGACAAGGGA
GTTGCTGTTTGGAAAATGTTTCACAGCAATCAAGATGAAGAGAGTAGGATGAAGGA
AATCTTGGAAATGCTACAAACTATTGAAGAACATGGCATTGGAGAGAAGAAAATTT
TCCACGGAGACAAGATTGGTTCACTTGAGATAGCATTTGGTTCAATGCTTTATTGGT
TGCAAATCCTTGAAGATATAGTGGGAGTGAAACTTTTTGATTCTCACAAATTCCCAG
GCCTACATGCGTGGTTTGAAAATTTCAAAAAAGCCCCTGTAATCGAAGAAAATCTTC
CTGATCGGGATGGAATTTCGCTCTTTTTCAAACGTCGCCGTGAAAATTTACTGGCTCC
TGCCTGA
>orange1.1t06017
(SEQ ID NO: 77)
ATGAGAGGGAATGATGTTTGGGCAAAGGGTGATTGGTTCACTTGGCTCATGAAGGA
CCCCAACTATAATGACATGTATCAATTTCGCAGCCTAATTGGAAG
>Cs7g14180
(SEQ ID NO: 78)
ATGTCCAAGGGAGAGGTTGTTCTTTCGGACTGTTGGGCTAGCCCATTTTGCTTGAGA
GCAAAGATTGCATTGGCTGAGAAAGGATTTGAATATGAAGCCAGAGCTGAGAACTT
GTTCGGGGGCAAGAGTGACCTGTTGCTCAAATCAAACCCCATCTACAAGAAAGTCC
CAGTTTTGCTACACGATGGGGAACCATTAGCAAAGATTGCATTGGCTGAGAAAGGA
GTTGAATATGAAGCCAGAGCTGAGAACTTGTTTGGGGGCAAGAGTGACCTGCTGCT
CAAATCAAACCCCATCTACAAGAAAGTCCCAGTTTTGCTACACGATGGGAAACCAA
TGTGTGAATCCACTATTATAGTTAACTACATTGACGAGACTTGGCCTTCGCCACCAC
TTCTCCCATCCTGTGCTTCTGAGAGAGCTAAGGCAAGGTTCTGGGCAGATTTTATTG
AGAAGGCATTCGATGCGGTCTGCAACATATGGAAAAGCAAGGGCGAGGTCCCCGAG
ACGGCGAAGAATGAATTTATAGAGATTTTGAAGCCACTTGAGGGAGCTTTAGGGGA
GAAGGATTTCTTTGGCGATAGCTTTGGATTCGTTGATGTCATTGCCATTCCACTGACT
TGTTGGTTTTACGCGGTTGAGAAATTTGGGGGATTTAAGCTTGAGAATGAGTGCCCC
AATTTCTCAGCATGGATGAACAAGTGCATGCAAAGAGATACTGTTGCCAGGATCCTT
CCAGACCCAGAAAAGGTCTACGAATTTGTTATCATGCTGAGGAACTTGTTTGGAATT
GAATAG
>orange1.1t03462
(SEQ ID NO: 79)
ATGGCTGAAGTGAAGCTTCTTGGTGGGGCAGTGAGTCCATTTGTTTACAGAGTGATA
TGGGCTCTGAAATTGAAAGGCGTACCATTTGAATTTGTTGAAGAAGATGTATTCTCA
AACGAAAAGAGTGCTTTGCTTCTGAAGTATAATCCAGTTCACAAGAAGATCCCAGTG
CTTGTTCATGGCGGAAAACCAGTTTGCGATTCCACGGTTGTTCTCGAATACATTGAA
GAGAAGTGGCCGTATAATCGCTTGTTGCCAAGTGACCCTTATGATAGAGCTCTTGCT
CGTTTTTGGATTAAATTTGGAGAAGACAAGGGAGTTGCTGTTTGGAGAATGTTTCAC
AGCAATCGAGATCAAGAGAGTAGAATGAAGGCAATCTTGGAAATGCTACAAACTAT
TGAAGAACATGGCCTAAGAGAGAAAATTTTCCATGGAGACAAGATTGGCTTACTTG
ACATAGCATTTGGTTCAATGCTTTATTGGTTGCAAATCCTTGAAGATATAGTGGGAG
TTAAACTTTTTGATCCTCACAAATTCCCAGGCTTGAATGCGTGGTTTGAAATTTTCAA
AAAAGCACCTGTAATTGAAGAAAATCTTCCTGATCAGGATGGAATTTCGCTCTTTTT
CAAACGTCGCCGTGAGAAGTGGCTGACTTCTGCCTGA

### Example 7: Prevent Overproduction of ROS

Preventing overproduction of ROS is achieved by editing the promoter or coding region of respiratory burst oxidative homolog D (RbohD) gene to reduce their induction by CLas. Specifically, phosphorylation of RbohD is required for its activation to produce ROS. Some of the phosphorylation sites of RbohD are specifically induced by CLas. Editing of those phosphorylation sites are also included as part of the editing of the RbohD coding region. Examples of phosphorylation sites for targeted editing include the codons of the RbohD gene coding for positions S31, S120, S150, S331, S335 and S33A of the RbohD amino acid sequence (SEQ ID NO:82). gRNA can be designed to make mutations at one or more these sites. Those skilled in the art will appreciate that other editing techniques can make mutations at these sites so as to block phosphorylation at said sites.

In another embodiment, using the techniques described herein, and in view of the knowledge in the art, the RbohD gene can be mutated to knock-down its expression.

In other embodiments, other Rboh genes may be targeted. These include but are not limited to the following genes with accession numbers in parentheses: to, CsRBOHB (Cs3g14240), CsRBOHD (Cs8g12000), and CsRBOHF (Cs5g02940). Using the techniques described herein, one or more of these genes can be targeted to knock-down their expression.

### Editing Technique

1.Two guide RNA are used to mutate RBOHD coding sequence using known CRISPR techniques, utilizing Cas9/gRNA. Many suitable gRNAs can be used. For example, sites of the target gene having GG in the downstream or CC in the upstream are suitable to design gRNAs as shown in the drawing below. Mutations are made at various locations and the

CCGTCGAGAAGCGGTATATAACGAGCTCGCCATCACGACCTCCGA...
/ /...GACAGGGATGAACAAGGAGTCTAAAGAATTTGCGGGTGAGCTAT
TCCGAGG

Note: CCG: PAM for the first gRNA. Sequence in red: the first gRNA.
AGG: PAM for the 2nd gRNA. Sequence in green: the first gRNA.
2. In addition, some site specific mutations (for example, phosphorylation sites) are engineered using citrus base editors. Mutations that abolish the induction by CLas without affecting its necessary function are elucidated by inoculating the edited plants with CLas through grafting and psyllid feeding. The effects of the editing of the promoter or coding region on CLas titers, ROS production, cell death in the phloem, and HLB symptom development are determined via the techniques described in Examples 1-5 above. Effects of editing on horticultural traits and fruit yield and quality is also determined.
3. Many suitable gRNAs can be used to edit the promoter region. For example, the sites have GG in the downstream or CC in the upstream are suitable to design gRNAs.

| RBOHD coding sequence | |
|---|---|
| >C.sinensis v1.1\|orange1.1g002525m.g\|RBOHD: scaffold00401: | |
| 70253..74476 reverse | |
| (SEQ ID NO: 80) | |
| ATGAGAAGCGAGGAGGGCAGAGGAGGCTATATTGAAAACAATTCAGATACTGAAA | |
| GTATTGGCAGTGACAAAAGCGCATTCAGCGGACCGCTTGCCAATAAACGAGCTTCC | |
| AAGAAAAGCGCCCgGTTCAATCTTCCGCCAGAAGCAGCAGTCACAAGCAGCAGTAA | |
| CAATAACGATGATGGTGGTTATGTAGAGATCACACTGGACATACGTGATGACTCTGT | |
| CGCAGTTCACAGCGTTCAGGGCGCACTTCATCAGAAGATCCTGAGTTGTCACTGTTG | |
| GCCAAGAAGACTTTGGAGATTAATAATAAATCTTCATCATTTCGGTCGTCTTTGTTCA | |
| GAAACACTTCCTCGCATATCAAGCAGGTCTCTCAAGAGCTCAAACGCTTTGCTTCCT | |
| TGACACGGCGACCCAGCAGCAGGCGTTTTGATAGGACTAAATCCGCTGCAGCTCAT | |
| GCTCTTAAAGGACTCAAGTTCATCACCACCAAGACGGGAGCTGCCGGAAATGGCTG | |
| GCCTGCCGTCGAGAAGCGGTATAACGAGCTCGCCATCACGACCTCCGACGGCCTTCT | |
| TCATTGCTCGATGTTCGGCGAATGCATAGGTACTTGCTGGCACAACGTCTTTAACAG | |
| TTCAGGAGATCTAGCTCAGATTATGAGAGCTAGGATCCTGATTCATGTCATATAGGA | |
| TCAATTACTTATTCTGTTCTTTTAtTTTTAATTTTGACAGGGATGAACAAGGAGTCTAA | |
| AGAATTTGCGGGTGAGCTATTCCGAGGACTTTGTAGAAAGCATAATATAAGTGGTG | |
| ATTCTATCAACAAAGCTCAGCTCaAgGAGTTTTGGGACCAGATTTCCGACGAGAGCTT | |
| TGACTCCAGGCTCCAAACTTTCTTTGACATGTAATTCATTTAACTACTTTATTAATTC | |
| ATATCAgTATAAAATCAAATTGTATATCTTGATTAATTATTTTTACTAACCGTCTTTG | |
| ATTAATTATTATATAGGGTCGACACAGACGCTGATGGAAGAATCACTGAAGAAGAA | |
| GTCAAAGAGGTAATTAATTAAGCAAGTTACGCATACATACTTGGTCAAAAACGCTA | |
| CATTTTAAGTTTATATATATGATTTCTATTTCTCTGTGTAGATTATTAGTCTGAGTGCT | |
| TCTGCAAACAAGCTCTCCAACATTCAGAAACAAGCAGAGGAATATGCAGCCATGAT | |
| TATGGAAGAATTAGACCCTGACAATGCCGGATACATCATGGTAATTTATTTTCTAAT | |
| TAATTAGATTAAAAAAGAAGaATTTTTTTaaAAAATTTTATTTAAATTGAGTACGAAA | |
| TTTGGCTTTTTGTCGCAGATTCATAACCTGGAAACATTGTTGTTGCAAGCTCCAAATC | |
| AATCTGTAAGAGTAGGAGACAGTCGAATTCTGAGTCAAATGTTGAGCCAGAAGCTG | |
| AAGCCAACCCAAGAAAACAACCCATTACGAAAATGTTATCAGAAGATTAAGtACTTT | |
| TTGATGGATAATTGGCAAAGGGTTTGGATTATGCTGTTGTGGCTTGGAATTGTTGGG | |
| GGTTTATTTGCCTACAAGTTTGTGCAGTACCGAAACAAGGCTGCTTATGATGTGATG | |
| GGCTATTGCGTTTGCATTGCTAAAGGAGGAGCAGAGACACTTAAATTCAACATGGCC | |
| TTGATATTATTACCAGTTTGCCGAAACACCATCACATGGCTCAGAAACAAAACTAAA | |
| TTAGGACTTGTTGTCCCTTTCGATGACAACTTAAATTTCCATAAGGTAATAATTAATA | |
| TCTTTAACGGATAAATCCAAATGTACAGTTTCAATGTCAtTTTTTTTTCtTTTTTAACCT | |
| TTGATATAAATAATAATATGCACGTGAACGTAGGTGATCGCGGTTGGAATTGGGATT | |
| GGAGTTATATTACACGGCGGAGCTCATCTAACCTGTGATTTCCCGCGACTCCTCCAC | |
| GCGACTGAAGAAGAATACGAGCCAATGAAGCCTTACTTTGGGGACGAGCAACCGGA | |
| TAATTACTGGTGGTTTGTAAAGGGAGTTGAAGGAGTAACAGGGATCATAATGGTGG | |
| TGTTAATGGTGATAGCATTCACTTTGGCAACGCCTTGGTTCAGGCGCAACAAGCTCA | |
| ATCTGCCAAAACCACTTAAAAGACTCACTGGATTCAATGCCTTTTGGTACTCACATC | |
| ATCTTTTCATCATTGTCTATACCCTTCTCATTGTCCATGGAATTAAACTCTATCTCACC | |
| AAGAAATGGTATCAGAAGACGGTAAGTGATCAATTACCtTTACCACCAGTTCCTTTaT | |
| TTTTCATTTTCATTTAACTTAATGTTAACTTCCTTTTTCAATTTATTTATCGACTGCAG | |
| ACATGGATGTATCTGGCTGTCCCGATGGTTCTTTATGCTTGTGAGAGATTGACTAGA | |
| GCTTTGAGATCAAGCATCGAGCCTGTCAAGATTCTCAAGGTTTTGCAACTATATATT | |
| CCTTAATTTTATTGTTAATTTCAGTGTGTTGCAAGTTAATTTGAGATTTCAGTGTGTT | |
| GCTAGTTAATTTGAGACATAACGAAATAATAAGATGAATTAATATTATGTGAAGGTT | |
| GCTGTTTATCCTGGAAATGTCTTGGCATTACACATGTCAAAACCACAAGGCTTCAAA | |
| TACAAGAGCGGACAGTACATGTTCGTAAATTGCGCTGCCGTCTCACCGTTCGAATGG | |
| TATTGAAATTGAAAAATTAGTTTATTATTATTTATTAAATTAGTTAATTAAACATTAA | |
| TTAATTAACAGCACTAAACTATCAATAATTGGTGTGACAGGCATCCGTTTTCCATCA | |
| CATCATCACCAGGAGATGACTACCTCAGCGTTCATATCCGAACTTTGGGCGATTGGA | |
| CTAGGCAGCTTAAGACAGTTTTCTCTGAGGTTTGTCAATTAAAATTCTAATTTCATTA | |
| TGCTTAATATTATTAATGTGCTCAGTTACTTAACtTTTTTTTTAATCTTATTTACACTAT | |
| TTTATGTGATTGTCTTTGGTTATTAGGTTTGCCAGCCTGCACCTGCTGGGAAGAGCGG | |
| TCTTCTTAGAGCAGAAAGGGAAAATAACTCTCGAGGGTATGTATTCTTTGTTTTTTCC | |
| TCTTTAAtAATTCATCAATGAAATTAATGTAAAATATTTGACAGAATGTTGACATAGA | |
| TAAGATAAGACAAATACCTATTTAATTACTTAACTaAAAAATAAAGTAAATCTCAAC | |
| TAATTTCGCTTATATTTCTGTTAGCGTTCTATCAAGATTCTGTTTGTGTCttTCtcCttttttaa | |
| TTaAAAtTTATATCTTGTGATGTAGCAGGTTTCCAAAGATATTAATAGACGGACCATA | |
| TGGAGCACCAGCTCAAGACTATAAAAAATATGATGTAGTGCTACTAGTGGGGCTTG | |
| GAATTGGGGCTACTCCCATGATCAGCATAGTCAAAGACATCATCAACAACATGAAA | |
| TCAGAAGACAACAATTTAGAGTCAGGATTAACAGTAAACAACAACAACAAGAATTC | |
| ATCATTCAATACAAGAAAAGCATACTTCTATTGGGTGACAAGGGAACAAGGTTCATT | |
| CGAGTGGTTCAAAGGCATAATGAATGAGGTGGCGGAGATGGATGAGAAGAGGGTA | |
| ATAGAATTACACAATTATTGCACAAGTGTGTACGAAGAAGGAGATGCCAGGTCAGC | |
| GCTTATAGCTATGCTTCAATCACTCCATCATGCAAAGAATGGTGTTGATGTTGTTTCT | |
| GGGACACGTGTCAAATCTCACTTTGCTAAGCCAAATTGGAGACAAGTTTACAAGAgG | |
| ATTGCTCTTCATCATCCTGATTCTAGAATTGGTCAGTTCTTTTTCTTCAACTTCGCTTT | |
| CACCTACCTCCACCCACTaTTCTTTTTCTTTGTgTACCACTAATTAATTTCCATTTTGCG | |
| TGGAATTAAATTTCTAAGATTTGTTCATATCAAAAATAATTTCTAAAATTTATTTATT | |
| TGATGTGTGCAGGAGTATTCTAtTGCGGTGCACCCGCACTAACAAAGGAGCTAAGAC | |
| AACTAGCCTCGGATTTctCCCACAAGACTTCCACTAAGTTTGAATTTCATAAAGAAAA | |
| TTTTTAAAATTAATTAATTTATAGCTTCATCAATTGATTAATAATATTATTCTTGAAC | |
| TTGGAAAGTTTTTTCTTTCTTTAGAAAGGaAGGTCCTCGGACATTAGTAGCCACTTGC | |
| AAATAGAAAATACGTACACTCAAATGGTCTGAC | |
| RBOHD promoter region | |
| (SEQ ID NO: 81) | |
| ACAATTTTCAATGGCCTCATTCAAGACACCCACCACTTATACTAGAAGTTAATGAGT | |
| GAAAGTGAGAGTAAAAAGGGAAAAGAGAAAAATACATGACTCAAAATATCGTACT | |
| TATTATCAAATTGAAAGTGGAAAGTGATGACACCCCACTTAGTTGATTTTTTTTAATT | |
| ATAATTTTATCTTTAAATTTAATTATCAAATAATAATTCATAAACTAAATGAATTTAT | |
| CTAATTCTCAATACATCATCAAGTTTTAACCTACAATAGTTTACATTTGCCTAAAATT | |
| AAATTAATTTTTTATAAAATTATAAAAAAAATTGGGACTTCTCTATAATATTGTTAA | |
| GAATATTATCCTTTCATCTATATTATTTTAAAAAATTTGATAATAAAAATAAGATAAT | |
| TAACTAATAATAACTTGTTTACGGACATTTCCCTCTCTCGTTTTGTGATGACGCCCCG | |
| CTTAGTTGATATTTTCTTAATAAAATAACAATTTTATCTTTAAATTAATGATCGAGTA | |
| ATAATTCATAAACTAAATGAACTTATCTAATTCTTAATTCATCATCATACTTTAATCT | |
| ATAATATTTTACGTATGCCTAAAATTAAAGTAACTTTTTATAAAATTATACAAAAAT | |
| TTGGGACATCTCTATAGTATTGTTAAGAATATTATTCTTTAACAAGGGATAGGATCA | |
| AGTAAAACTTGATCCAAAGGTTAAAGTGATCCTAATGCAACAATTAGGCACAATTTA | |
| AGTGAAAATTAGTTATTTCTCTAAAAAATTCAGCTATATAAACAGTCAATGTGTCAT | |
| TTTAATTGGATTACACAATTAATGAGAGCAAACACAGATTATTTTGTAGATCCTCCC | |
| AGAAGGACATCCTAGCCCATTCAAAAACAGTTTCATTTTCATTTTTCTTTATTTTCTC | |
| ATGAACCAATCTTGATTTGAGCATCATAGTGTTTTTGGAAGGCACTAAACCGATGCT | |
| TGGAGCTTTTAGAGCTCAAAGACAATGAAGGATAGATCCGGTCATTTCGCAATCAA | |
| GATTTCTTTATCTTGAATTGGCCAAAGCTAAATTTCTTAATCAAAACTAGGGGTGGC | |
| AATATAGGTCAAGATTCGTTCATTCGATTCGATTTAATATGAATTCAATGGATTTGG | |
| GTTTGAAAAATTAATTTGTTTAATAAATTGGTCAAATTTGATTCGATTTGCTAAATAA | |
| ACGAATTAAATATGGGATTAAGCTCACAAATTGTTTAAAATTCATTTATTGAACAAT | |
| GCAATAAAAATAAGAAATCATACAGAAAAGTCACTATGGTTCAAAAATATTTATTTT | |
| AAGGATTTTAATTCTTATTATTTGTTTTTTTTAATGGATTGGTAATTTGAAGTTTAGA | |
| GCTGTGGAGTTAAGATGTTTAAAACTTTAAATATTGTAAGGATGATAAATCTATGAT | |
| AGTTTATTTTTCTTAATTTTCGTTGTTTACTAATTATTATTTGATATATAATATGATAA | |
| TTCTAATTGAATGAATTTAAACAAATCCAAATCTAATTCAATTTATTTATTCATTTGA | |
| TTAAATGAATAAATGAATATTCGTTTAAAAAACGAATCGATATCTGAATAAGCTATG | |
| ATTAGTTTAATAAACGAATTGAATTTGAATCTAATTATTTTGACCCAATTCATTTAAT | |
| ATTCAACACGAATTCTTATGAATTCGTTTTGGCCTCCAAATCAAAACCCTTTCAAGTT | |
| GAATTGTGAACATTCCAAAAGTCAATGATGAAGCCATAGAATCTTGCTATACATCCT | |
| ATTTACCAGCAAACAATGATTTCGTATTTTTAGTAACCGTATATTTCCTTTTATATAC | |
| TATAATTTTTTGTGTTAAAAAAAAAAAAAAATGAAAATTTTCTGGTCCTAAGGTCAG | |
| GTTTGACCAGCATATCACTTTCCTACAAGCTTCTTCTGAATCACGTTTACATTCAACT | |
| TTCTTTATATATATATATATATATATATAAACCATATGATCATCAAAT | |
| Amino Acid Sequence of RBOHD | |
| SEQ ID NO: 82 | |
| MKKMRSEEGR GGYIENNSDT ESIGSDKSAF SGPLANKRAS KKSARFNLPP EAAVTSSSNN | 60 |
| NDDGGYVEIT LDIRDDSVAV HSVQGALHHE DPELSLLAKK TLEINNKSSS FRSSLFRNTS | 120 |
| SHIKQVSQEL KRFASLTRRP SSRRFDRTKS AAAHALKGLK FITTKTGAAG NGWPAVEKRY | 180 |
| NELAITTSDG LLHCSMFGEC IGMNKESKEF AGELFRGLCR KHNISGDSIN KAQLKEFWDQ | 240 |
| ISDESFDSRL QTFFDMVDTD ADGRITEEEV KEIISLSASA NKLSNIQKQA EEYAAMIMEE | 300 |
| LDPDNAGYIM IHNLETLLLQ APNQSVRVGD SRILSQMLSQ KLKPTQENNP LRKCYQKIKY | 360 |
| FLMDNWQRVW IMLLWLGIVG GLFAYKFVQY RNKAAYDVMG YCVCIAKGGA ETLKFNMALI | 420 |
| LLPVCRNTIT WLRNKTKLGL VVPFDDNLNF HKVIAVGIGI GVILHGGAHL TCDFPRLLHA | 480 |
| TEEYEPMKPY FGDEQPDNYW WFVKGVEGVT GIIMVVLMVI AFTLATPWFR RNKLNLPKPL | 540 |
| KRLTGFNAFW YSHHLFIIVY TLLIVHGIKL YLTKKWYQKT TWMYLAVPMV LYACERLTRA | 600 |
| LRSSIEPVKI LKVAVYPGNV LALHMSKPQG FKYKSGQYMF VNCAAVSPFE WHPFSITSSP | 660 |
| GDDYLSVHIR TLGDWTRQLK TVFSEVCQPA PAGKSGLLRA ERENNSRGFP KILIDGPYGA | 720 |
| PAQKYKKYDV VLLVGLGIGA TPMISIVKDI INNMKSEDNN LESGLTVNNN NKNSSFNTRK | 780 |
| AYFYWVTREQ GSFEWFKGIM NEVAEMDEKR VIELHNYCTS VYEEGDARSA LIAMLQSLHH | 840 |
| AKNGVDVVSG TRVKSHFAKP NWRQVYKRIA LHHPDSRIGV FYCGAPALTK ELRQLASDFS | 900 |
| HKTSTKFEFH KENF | |

### Example 8: Other Gene Sequences that can be Modified to Improve Resistance to CLas Infection

orange1.1t03332.1 (NBS-LRR), orange1.1t04682.1 (NBS-LRR), orange1.1t05285.1 (PLCP, cysteine protease-like protein), Cs6g22310.1 (lectin), orange1.1t05183.1 (Leucine-rich repeat receptor-like protein kinase), Cs1g05340.1 (LRR-XII), Cs9g13810.1 (RLCK-XII/XIII), and Cs6g09910.5 (MAPKKK, Raf31), which were present in most HLB susceptible accessions (67-83%), but were absent in all HLB resistant accessions. Accordingly, in another embodiment, one or more of the foregoing genes can be edited by a gene editing technique to knock down their expression. In a specific embodiment, provided is a method of generating a modified plant comprising resistance or tolerance to infection by a bacterial species from the genus Ca. Liberibacter; the method comprising the steps of: (a) introducing a genetic modification into the genome of a plant cell, wherein the modification is to a gene or regulatory element thereof, wherein the modification confers resistance or tolerance to Ca Liberibacter infection in the plant relative to a plant of the same variety lacking the modification; and (b) regenerating the modified plant from said plant cell or a progenitor cell thereof, wherein said the plant comprises said modification, wherein the gene comprises orange1.1t03332.1 (NBS-LRR), orange1.1t04682.1 (NBS-LRR), orange1.1t05285.1 (PLCP, cysteine protease-like protein), Cs6g22310.1 (lectin), orange1.1t05183.1 (Leucine-rich repeat receptor-like protein kinase), Cs1g05340.1 (LRR-XII), Cs9g13810.1 (RLCK-XII/XIII), and Cs6g09910.5 (MAPKKK, Raf31). Plants comprising at least one plant cell comprising modification that knocks down expression of one or more of these genes is also provided.

Cs2g10550.1 (Leucine-rich repeat receptor-like protein kinase), and Cs1g05370.1 (Serine-threonine protein kinase, plant-type) were present in 75% of four HLB tolerant accessions, but absent in the 6 HLB susceptible accessions. Accordingly, in another embodiment, provided is a method of generating a modified plant comprising resistance or tolerance to infection by a bacterial species from the genus Ca. Liberibacter; the method comprising the steps of: (a) introducing a genetic modification into the genome of a plant cell, wherein the modification is to an endogenous gene or regulatory element thereof, wherein the endogenous gene comprises Cs2g10550.1 (Leucine-rich repeat receptor-like protein kinase), and/or Cs1g05370.1 (Serine-threonine protein kinase, plant-type), and wherein the modification confers resistance or tolerance to Ca Liberibacter infection in the plant relative to a plant of the same variety lacking the modification; and (b) regenerating the modified plant from said plant cell or a progenitor cell thereof, wherein said the plant comprises said modification. The modification may comprise a constitutive promoter (such as AtSUC2 promoter or CaMV35 promoter) operatively linked to the gene thereby inducing overexpression of the gene. Introducing step (a) may involve transfecting a plant cell with an expression vector, such as via a CTV vector. Another method relates to a method of increasing resistance or tolerance of a citrus plant to infection by a bacterial species from the genus Ca. Liberibacter; the method comprising introducing an expression vector into a plant cell of the plant, wherein the expression vector comprises a gene, or regulatory element thereof, optionally with at least one modification, wherein the gene comprises Cs2g10550.1 (Leucine-rich repeat receptor-like protein kinase), and/or Cs1g05370.1 (Serine-threonine protein kinase, plant-type).

### REFERENCES

1. Adapa, D., Sai, Y., Anand, S., Mehaboobi, S. & Aramalla, E. A Brief Review on Immune Mediated 605 Diseases. J Clin Cell Immunol, S11:001. (2011).
2. Barth, K., Remick, D. G. & Genco, C. A. Disruption of immune regulation by microbial pathogens and resulting chronic inflammation. J Cell Physiol 228, 1413-1422, doi:10.1002/jcp.24299 (2013).
3. Speth, E. B., Lee, Y. N. & He, S. Y. Pathogen virulence factors as molecular probes of basic plant cellular functions. Curr Opin Plant Biol 10, 580-586, doi:10.1016/j.pbi.2007.08.003 (2007).
4. Hu, Y. et al. Lateral organ boundaries 1 is a disease susceptibility gene for citrus bacterial canker disease. Proc Natl Acad Sci USA 111, E521-529, doi:10.1073/pnas.1313271111 (2014).
5. Chatterjee, S., Almeida, R. P. & Lindow, S. Living in two worlds: the plant and insect lifestyles of Xylella fastidiosa. Annu Rev Phytopathol 46, 243-271, doi:10.1146/annurev.phyto.45.062806.094342 (2008).
6. Bové, J. M. Huanglongbing: a destructive, newly-emerging, century-old disease of citrus. J. Plant Path., 7-37 (2006).
7. Thapa, S. P. et al. Genome-wide analyses of Liberibacter species provides insights into evolution, phylogenetic relationships, and virulence factors. Mol Plant Pathol, doi:10.1111/mpp.12925 (2020).
8. Clark, K. et al. An effector from the Huanglongbing-associated pathogen targets citrus proteases. Nat Commun 9, 1718, doi:10.1038/s41467-018-04140-9 (2018).
9. Pang, Z. et al. Citrus CsACD2 Is a Target of Candidatus Liberibacter asiaticus in Huanglongbing Disease. Plant Physiol 184, 792-805, doi:10.1104/pp. 20.00348 (2020).
10. Li, J. et al. ‘Candidatus Liberibacter asiaticus’ Encodes a Functional Salicylic Acid (SA) Hydroxylase That Degrades SA to Suppress Plant Defenses. Mol Plant Microbe Interact 30, 620-630, doi:10.1094/MPMI-12-16-0257-R (2017).
11. Luna, E. et al. Callose deposition: a multifaceted plant defense response. Mol Plant Microbe Interact 24, 183-193, doi:10.1094/MPMI-07-10-0149 (2011).
12. Raiol-Junior, L. L., Cifuentes-Arenas, J. C., de Carvalho, E. V., Girardi, E. A. & Lopes, S. A. Evidence That ‘Candidatus Liberibacter asiaticus’ Moves Predominantly Toward New Tissue Growth in Citrus Plants. Plant Dis 105, 34-42, doi:10.1094/PDIS-01-20-0158-RE (2021).
13. Otero, S. & Helariutta, Y. Companion cells: a diamond in the rough. J Exp Bot 68, 71-78, doi:10.1093/jxb/erw392 (2017).
14. Kaur, P., Gonzalez, P., Dutt, M. & Etxeberria, E. Identification of sieve elements and companion cell protoplasts by a combination of brightfield and fluorescence microscopy. Appl Plant Sci 6, e01179, doi:10.1002/aps3.1179 (2018).
15. Kim, J. S., Sagaram, U. S., Burns, J. K., Li, J. L. & Wang, N. Response of sweet orange (Citrus sinensis) to ‘Candidatus Liberibacter asiaticus’ infection: microscopy and microarray analyses. Phytopathology 99, 50-57, doi:10.1094/PHYTO-99-1-0050 (2009).
16. Koh, E. J. et al. Callose deposition in the phloem plasmodesmata and inhibition of phloem transport in citrus leaves infected with “Candidatus Liberibacter asiaticus”. Protoplasma 249, 687-697, doi:10.1007/s00709-011-0312-3 (2012).
17. Rong, W., Feng, F., Zhou, J. & He, C. Effector-triggered innate immunity contributes Arabidopsis resistance to Xanthomonas campestris. Mol Plant Pathol 11, 783-793, doi:10.1111/j.1364-3703.2010.00642.x (2010).
18. Wang, Y., Li, X., Fan, B., Zhu, C. & Chen, Z. Regulation and Function of Defense-Related Callose Deposition in Plants. Int J Mol Sci 22, doi:10.3390/ijms22052393 (2021).
19. McLennan, H. R. & Degli Esposti, M. The contribution of mitochondrial respiratory complexes to the production of reactive oxygen species. J Bioenerg Biomembr 32, 153-162, doi:10.1023/a:1005507913372 (2000).
20. Li, J. et al. Residue dynamics of streptomycin in citrus delivered by foliar spray and trunk injection and effect on ‘Candidatus Liberibacter asiaticus’ titer. Phytopathology, doi:10.1094/phyto-09-20-0427-r (2020).
21. Gechev, T. S., Van Breusegem, F., Stone, J. M., Denev, I. & Laloi, C. Reactive oxygen species as signals that modulate plant stress responses and programmed cell death. Bioessays 28, 1091-1101, doi:10.1002/bies.20493 (2006).
22. Montillet, J. L. et al. Fatty acid hydroperoxides and H₂O₂ in the execution of hypersensitive cell death in tobacco leaves. Plant Physiol 138, 1516-1526, doi:10.1104/pp. 105.059907 (2005).
23. Kumar, N., Ebel, R. C. & Roberts, P. D. Superoxide dismutase activity in kumquat leaves infected with Xanthomonas axonopodis pv. citri. The Journal of Horticultural Science and Biotechnology 86, 62-68, doi:10.1080/14620316.2011.11512726 (2011).
24. Teper, D., Xu, J., Li, J. & Wang, N. The immunity of Meiwa kumquat against Xanthomonas citri is associated with a known susceptibility gene induced by a transcription activator-like effector. PLoS Pathog 16, e1008886, doi:10.1371/journal.ppat.1008886 (2020).
25. Torres, M. A., Jones, J. D. & Dangl, J. L. Reactive oxygen species signaling in response to pathogens. Plant Physiol 141, 373-378, doi:10.1104/pp. 106.079467 (2006).
26. Guénal, I., Sidoti-de Fraisse, C., Gaumer, S. & Mignotte, B. Bcl-2 and Hsp27 act at different levels to suppress programmed cell death. Oncogene 15, 347-360, doi:10.1038/sj.onc.1201182 (1997).
27. Claeys, H., Van Landeghem, S., Dubois, M., Maleux, K. & Inzd, D. What Is Stress? Dose-Response Effects in Commonly Used in Vitro Stress Assays. Plant Physiol 165, 519-527, doi:10.1104/pp. 113.234641 (2014).
28. Torres, M. A., Dangl, J. L. & Jones, J. D. Arabidopsis gp91phox homologues AtrbohD and AtrbohF are required for accumulation of reactive oxygen intermediates in the plant defense response. Proc Natl Acad Sci USA 99, 517-522, doi:10.1073/pnas.012452499 (2002).
29. Levine, D. S. Immune modulating therapies for idiopathic inflammatory bowel diseases. Adv Pharmacol 25, 171-234, doi:10.1016/s1054-3589(08)60432-9 (1994).
30. Wu, D., Lewis, E. D., Pae, M. & Meydani, S. N. Nutritional Modulation of Immune Function: Analysis of Evidence, Mechanisms, and Clinical Relevance. Front Immunol 9, 3160, doi:10.3389/fimmu.2018.03160 (2018).
31. Campbell, N. K., Fitzgerald, H. K. & Dunne, A. Regulation of inflammation by the antioxidant haem oxygenase 1. Nat Rev Immunol 21, 411-425, doi:10.1038/s41577-020-00491-x (2021).
32. Huot, B., Yao, J., Montgomery, B. L. & He, S. Y. Growth-defense tradeoffs in plants: a balancing act to optimize fitness. Mol Plant 7, 1267-1287, doi:10.1093/mp/ssu049 (2014).
33. Ma, X. et al. Dual and Opposing Roles of Xanthine Dehydrogenase in Defense-Associated Reactive Oxygen Species Metabolism in Arabidopsis. Plant Cell 28, 1108-1126, doi:10.1105/tpc.15.00880 (2016).
34. Chen, S. et al. NtMYB4 and NtCHS1 Are Critical Factors in the Regulation of Flavonoid Biosynthesis and Are Involved in Salinity Responsiveness. Front Plant Sci 10, 178, doi:10.3389/fpls.2019.00178 (2019).
35. Furman, D. et al. Chronic inflammation in the etiology of disease across the life span. Nat Med 25, 1822-1832, doi:10.1038/s41591-019-0675-0 (2019).
36. Kotas, M. E. & Medzhitov, R. Homeostasis, inflammation, and disease susceptibility. Cell 160, 816-827, doi:10.1016/j.cell.2015.02.010 (2015).
37. Canales, E. et al. ‘Candidatus Liberibacter asiaticus’, Causal Agent of Citrus Huanglongbing, Is Reduced by Treatment with Brassinosteroids. PLoS One 11, e0146223, doi:10.1371/journal.pone.0146223 (2016).
38. Zhang, Z., Lynch, J. P., Zhang, B. & Wang, Q. in Plant Macronutrient Use Efficiency (ed TakehiroKamiya Mohammad Anwar Hossain, David J. Burritt, Lam-Son Phan Tran, Toru Fujiwara) 245-265 (Academic Press, 2017).
39. Tavanti, T. R. et al. Micronutrient fertilization enhances ROS scavenging system for alleviation of abiotic stresses in plants. Plant Physiol Biochem 160, 386-396, doi:10.1016/j.plaphy.2021.01.040 (2021).
40. Yuan, X. et al. Region-wide comprehensive implementation of roguing infected trees, tree replacement, and insecticide applications successfully controls citrus HLB. Phytopathology, doi:10.1094/phyto-09-20-0436-r (2020).
41. Hijaz, F., Al-Rimawi, F., Manthey, J. A. & Killiny, N. Phenolics, flavonoids and antioxidant capacities in. Plant Signal Behav 15, 1752447, doi:10.1080/15592324.2020.1752447 (2020).
42. Deng, H. et al. Phloem Regeneration Is a Mechanism for Huanglongbing-Tolerance of “Bearss” Lemon and “LB8-9” Sugar Belle. Front Plant Sci 10, 277, doi:10.3389/fpls.2019.00277 (2019).

While various disclosed embodiments have been described above, it should be understood that they have been presented by way of example only, and not limitation. Numerous changes to the subject matter disclosed herein can be made in accordance with this Disclosure without departing from the spirit or scope of this Disclosure. In addition, while a particular feature may have been disclosed with respect to only one of several implementations, such feature may be combined with one or more other features of the other implementations as may be desired and advantageous for any given or particular application.

Thus, the breadth and scope of the subject matter provided in this Disclosure should not be limited by any of the above explicitly described embodiments. Rather, the scope of this Disclosure should be defined in accordance with the following claims and their equivalents. The terminology used herein is for the purpose of describing particular embodiments only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The teachings of any patents, patent applications, technical or scientific articles or other references are incorporated herein in their entirety to the extent not inconsistent with the teachings herein.

What is claimed is:

1. A plant comprising plant cells comprising a modification to a gene, or regulatory element thereof, wherein the gene encodes an antioxidant enzyme, and wherein the modification confers resistance or tolerance to Ca Liberibacter infection in the plant relative to a plant of the same variety lacking the modification, wherein the gene encoding the antioxidant enzyme optionally comprises at least one of SEQ ID NOs: 3-79.

2. The plant of claim 1, wherein the antioxidant enzyme is selected from a group consisting of superoxide dismutase, catalases, glutathione peroxidases, ascorbate peroxidase, glutathione reductase, and glutathione S-transferase.

3. The plant of claim 1 or 2, wherein the plant is citrus.

4. The plant of any of claims 1-3, wherein the modification comprises operatively linking a constitutive promoter to the gene thereby inducing overexpression of the gene.

5. The plant of claim 4, wherein the constitutive promoter is a 35S promoter or a phloem specific AtSUC2 promoter.

6. The plant of any of claims 1-5, wherein said modification comprises a deletion, a substitution, or an insertion.

7. The plant of claim 6, wherein the modification causes activation of expression in response to CLas infection.

8. The plant of claim 7, wherein the modification alters or eliminates a function of the regulatory element thereof.

9. A plant comprising plant cells comprising a modification to a gene, or regulatory element thereof, wherein the gene is a respiratory burst oxidative homolog D (RbohD), and wherein the modification confers resistance or tolerance to Ca Liberibacter infection in the plant relative to a plant of the same variety lacking the modification, wherein the RbhoD optionally comprises SEQ ID NO:80.

10. The plant of claim 9, wherein the plant is citrus.

11. The plant of claim 9 or 10, wherein said modification comprises a deletion, a substitution, or an insertion of the promoter or coding region such that expression of RbohD is knocked down and/or phosphorylation of RbohD is reduced.

12. The plant of claim 11, wherein the modification causes reduction of expression in response to CLas infection.

13. The plant of claim 11, wherein the modification alters or eliminates a function of the regulatory element thereof.

14. A seed that produces the plant of any of claims 1-12.

15. A plant part of the plant of any of claims 1-12

16. A method of producing a commodity plant product, wherein the method comprises collecting the commodity plant product from the plant of any of claims 1-12.

17. A commodity plant product that is produced by the method of claim 16, wherein the commodity plant product comprises at least one cell of said citrus plant.

18. A method of generating a modified plant comprising resistance or tolerance to infection by a bacterial species from the genus Ca. Liberibacter; the method comprising the steps of:
(a) introducing a genetic modification into the genome of a plant cell, wherein the modification is to an endogenous gene or regulatory element thereof, wherein the endogenous gene encodes an antioxidant enzyme, and wherein the modification confers resistance or tolerance to Ca Liberibacter infection in the plant relative to a plant of the same variety lacking the modification; and
(b) regenerating the modified plant from said plant cell or a progenitor cell thereof, wherein said the plant comprises said modification, wherein the gene encoding the antioxidant enzyme optionally comprises at least one of SEQ ID NOs: 3-79.

19. The method of claim 18, wherein the polypeptide is selected from a group consisting of superoxide dismutase, catalases, glutathione peroxidases, ascorbate peroxidase, glutathione reductase, and glutathione S-transferase.

20. The method of claim 18 or 19, wherein the plant is citrus.

21. A method of generating a modified plant comprising resistance or tolerance to infection by a bacterial species from the genus Ca. Liberibacter; the method comprising the steps of:
(a) introducing a genetic modification into the genome of a plant cell, wherein the modification is to an RbohD gene or regulatory element thereof, wherein the modification confers resistance or tolerance to Ca Liberibacter infection in the plant relative to a plant of the same variety lacking the modification; and
(b) regenerating the modified plant from said plant cell or a progenitor cell thereof, wherein said the plant comprises said modification, wherein the RbohD optionally comprises SEQ ID NO:80.

22. The method of claim 21, wherein the plant is citrus.

23. The method of any of claim 21 or 22, wherein the modification is to the promoter of the RbohD gene so as to reduce expression in response to CLas infection, or a modification to a coding region of the RbohD gene that knocks-down expression.

24. The method of any of claims 18-23, wherein step (a) comprises a genome-editing technique.

25. The method of claim 24, wherein the genome-editing technique comprises a nuclease, wherein the nuclease introduces a single-strand DNA break or a double-strand DNA break.

26. The method of claim 24 or 25, wherein the genome-editing technique comprises a TALEN, a ZFN, meganuclease, or a CRISPR/Cas system.

27. The method of claims 18-26, wherein the plant comprises resistance to HLB.

28. A plant comprising resistance to Ca. Liberibacter infection produced by the method of any of claims 18-26.

29. A method of increasing resistance or tolerance of a citrus plant to infection by a bacterial species from the genus Ca. Liberibacter; the method comprising introducing an expression vector into a plant cell of the plant, wherein the expression vector comprises a gene, or regulatory element thereof, optionally with at least one modification, wherein the gene encodes a citrus antioxidant enzyme and wherein the gene encoding the antioxidant enzyme optionally comprises at least one of SEQ ID NOs: 3-79

30. The method of claim 29, wherein the polypeptide is selected from a group consisting of superoxide dismutase, catalases, glutathione peroxidases, ascorbate peroxidase, glutathione reductase, and glutathione S-transferase.

31. The method of claim 29 or 30, wherein the plant is citrus.

32. The method of any of claims 29-31, wherein the modification comprises a constitutive promoter operatively linked to the gene thereby inducing overexpression of the gene.

33. The method of claim 32, wherein the constitutive promoter is a 35S promoter or a phloem specific AtSUC2 promoter.

34. The method of any of claims 29-33, wherein said modification comprises a deletion, a substitution, or an insertion.

35. The method of any of claims 29-34, wherein the modification causes activation of expression in response to CLas infection.

36. The method of any of claims 29-35, wherein the expression vector is delivered to the plant cell via a CTV vector.

37. A method of generating a modified plant comprising resistance or tolerance to infection by a bacterial species from the genus Ca. Liberibacter; the method comprising the steps of:
(a) introducing a genetic modification into the genome of a plant cell, wherein the modification is to an RbohB gene or regulatory element thereof or RbohF gene or regulatory element thereof, wherein the modification confers resistance or tolerance to Ca Liberibacter infection in the plant relative to a plant of the same variety lacking the modification; and
(b) regenerating the modified plant from said plant cell or a progenitor cell thereof, wherein said the plant comprises said modification.

38. The method of claim 37, wherein the plant is citrus, and the RbohB gene or RbohF genes are Cs3g14240 or Cs5g02940, respectively.

39. A plant produced by the method of claim 37 or 38.

40. A plant comprising plant cells comprising a modification to a gene, or regulatory element thereof, wherein the gene is a respiratory burst oxidative homolog B (RbohB) or respiratory burst oxidative homolog F (RbohF), and wherein the modification confers resistance or tolerance to Ca Liberibacter infection in the plant relative to a plant of the same variety lacking the modification.

41. The plant of claim 40, wherein the plant is citrus.

42. The plant of claim 40 or 41, wherein said modification comprises a deletion, a substitution, or an insertion of the promoter or coding region such that expression of RbohB or RbohF is knocked down.
